(19) United States
(12) Patent Application Publication
(10) Pub. No.: US 2012/0202877 A1
(43) Pub. Date: Aug. 9, 2012

(54) ANTI-INFLUENZA AGENTS

(76) Inventors: Mark Von Itzstein, Palm Beach (AU); Jeffrey Clifford Dyason, Palm Beach (AU); Robin Thomson, Chevron Island (AU); Santosh Rudrawar, Labrador (AU); Mauro Pascolutti, Bundall (AU)

(21) Appl. No.: 13/384,198

(22) PCT Filed: Jul. 16, 2010
(86) PCT No.: PCT/AU2010/000905

§ 371 Date: Apr. 25, 2012

(30) Foreign Application Priority Data
Jul. 16, 2009 (AU) 200903329

(51) Int. Cl. A61K 31/351; A61P 31/16; C07D 309/30

(52) U.S. Cl. 514/460; 549/420

(57) ABSTRACT

The present invention relates to compounds that selectively inhibit influenza A virus group (1) sialidases and are therefore potential anti-influenza agents.

### TECHNICAL FIELD

The present invention relates compounds that inhibit influenza A virus sialidases and are therefore potential anti-influenza agents.

### BACKGROUND ART

Infection by influenza viruses, in particular type A viruses, has had a significant impact on human health over the centuries, including three pandemics in the 20th century (Horimoto and Kawaoka, 2001). Vaccines are available against influenza virus but are effective only against particular strains. Until recently the drugs of choice for the treatment of influenza A virus infection were the adamantane-based M2 ion channel protein inhibitors, Rimantadine and Amantadine (Douglas, 1990). However, both drugs have been reported not only to have significant side-effects, but also lead to the rapid emergence of drug resistant influenza viral strains.

Since 1999, inhibitors of the viral surface enzyme sialidase (neuraminidase, NA) have been available for treatment and prophylaxis of influenza A and B virus infection. The sialidase plays a key role in the life cycle of influenza viruses, facilitating the release of virus progeny from the infected cell surface by cleaving the cell-surface virus attachment ligands. Inhibition of the sialidase activity leads to clumping of the virus progeny at the cell surface, resulting in diminished propagation of infection (Palese and Compans, 1976). Despite the rapid antigenic mutability of the sialidase, the critical amino acids of the sialidase active site (both those contacting the substrate and the supporting framework residues) were found to be highly conserved in all strains of influenza A and B virus sialidase examined to the mid 1980s (Varghese et al., 1992). This observation led to the design and development of a number of potent and selective inhibitors of influenza virus sialidase (Rich et al., 2007), two of which, zanamivir (von Itzstein et al., 1993) and oseltamivir carboxylate (Kim et al., 1997), are now on the market. Both inhibitors are sub-nanomolar inhibitors of both influenza A and B virus sialidases. Oseltamivir carboxylate is currently recommended by the WHO as the primary antiviral treatment for pharmacological management of influenza A(H1N1) virus infection (treatment and prophylaxis) (WHO Guidelines, August 2007), and has been stockpiled by governments around the world as part of their preparedness plans for an outbreak of pandemic influenza. However, strains of influenza virus resistant to oseltamivir carboxylate have been reported, both in oseltamivir-treated patients (reviewed in Reece, 2007), and recently in circulating strains in wild bird populations. With the spectre of the decreased efficacy, through resistance development, of the most widely used sialidase inhibitor, work towards development of next generation sialidase inhibitors is of importance.

There are two phylogenetically distinct groups of influenza A virus sialidases—group 1 (N1, N4, N5, N8) and group 2 (N2, N3, N6, N7, N9) (Russell et al., 2006). Influenza A virus strains infecting humans in the 20th century carried either N1 (group 1) or N2 (group 2) sialidases (although there have been reports of a small number of people infected with N7 viral strains) (Horimoto and Kawaoka, 2001). An influenza A virus strain carrying a group 1 sialidase caused the most devastating influenza pandemic of the 20th century [1914-1915 (H1N1). Over the past few years an avian influenza virus strain of H5N1 strain has been of global concern and, more recently, an influenza pandemic involving an H1N1 strain has been declared. The two groups of sialidases have recently been shown crystallographically to be structurally distinct (Russell et al. 2006). Group 1 sialidases have significant conformational flexibility in the so-called ‘150-loop’, which has always been seen in the ‘closed’ conformation in group 2 sialidases. In group 1 sialidases, in the apo structure (no inhibitor or substrate bound) the 150-loop is seen in the ‘open’ conformation resulting in a larger potential active/binding site compared to group 2 sialidases.

Structure-based design of influenza virus sialidase inhibitors reported to date has been carried-out using the X-ray crystal structures of sialidases from influenza A virus group 2 (N2 and N9) sialidases, and influenza B sialidase. These inhibitors show comparable inhibition of both influenza A virus group 1 and 2 sialidases, however none were designed to exploit binding to the structure of group 1 sialidases with the ‘open’ conformation of the 150 loop.

### SUMMARY OF THE INVENTION

The present invention relates to novel compounds which bind to influenza A virus group 1 sialidases with the 150-loop in the ‘open’ conformation. Consistent with this observation; the compounds are selective inhibitors of influenza A virus group 1 sialidases.

According to a first aspect the present invention provides a compound of general formula (I) which is a selective inhibitor of influenza A virus group 1

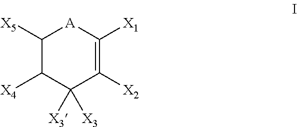

or a pharmaceutically acceptable salt, ester or prodrug thereof, wherein

A is O, S or NR₁;

where R₁ is hydrogen, optionally substituted alkyl, optionally substituted aryl, optionally substituted acyl or optionally substituted sulfonyl;

X₁ is CO₂H, P(O)(OH)₂, NO₂, SO₂H, SO₃H, —C(O)NHOH or tetrazole;

X₂ is alkyl, aralkyl, alkenyl, alkynyl, optionally substituted alkyl, optionally substituted aralkyl, optionally substituted alkenyl, optionally substituted alkynyl, OR₂, SR₂, NR₂R₂′, or substituted triazole,

where R₂ and R₂′ are selected independently from optionally substituted acyl, optionally substituted sulfonyl, alkyl, alkenyl, alkynyl, optionally substituted alkyl, or optionally substituted alkenyl,

or R₂′ is hydrogen;

X₃ and X₃′ are selected independently from hydrogen. R₃, halogen, CN, OR₃, NR₃R₃′, NHC(NR₃)N(R₃)₂, N₃, SR₃, —O—CH₂—C(O)—NR₃R₃′, —O—CH₂—C(NH)—NR₃R₃′, —O—CH₂—C(S)—NR₃R₃′

and optionally substituted triazole,

or X₃ and X₃′ together are ═O, ═N—OR₃, or ═CH—R₃

where R₃ and R₃′ are selected independently from hydrogen, optionally substituted acyl, optionally substituted sulfonyl, alkyl, aralkyl, alkenyl, alkynyl, heteroalkyl, heterocyclyl, optionally substituted alkyl, optionally substituted aralkyl, optionally substituted alkenyl, —C(O)R₄ and —S(O)₂R₈,

where R₈ is selected from optionally substituted alkyl and optionally substituted alkenyl;

X₄ is NR₄R₄′, OR₄, SR₄, CH₂C(O)R₄, CH₂C(O)OR₄, CH₂C(O)NR₄R₄′, CHR₄NO₂, CHR₄CN, CHR₄R₄′, or CH₂NHR₄,

where R₄ and R₄′ are selected independently from hydrogen, optionally substituted acyl, optionally substituted thioacyl, optionally substituted sulfonyl, alkyl, alkenyl, alkynyl, optionally substituted alkyl, optionally substituted aralkyl, optionally substituted alkenyl, optionally substituted heteroaryl, and optionally substituted heterocyclyl;

X₅ is optionally substituted alkyl, optionally substituted aralkyl, optionally substituted alkenyl, optionally substituted alkynyl, optionally substituted heteroaryl, optionally substituted heterocyclyl, —C(O)R₅, —CO₂R₅, —C(O)NR₅R₅′, —P(O)(OR₅)(OR₅′), —P(O)(OR₅)(NR₅R₅′), —P(O)(NR₅R₅′)₂, CN, OR₆, azide, NHR₆, NR₆R₆′, SR₆, or optionally substituted triazole,

where R₅ and R₅′ are independently selected from hydrogen, optionally substituted alkyl, optionally substituted alkenyl, optionally substituted aryl, or heteroaryl, and

R₆ and R₆′ are independently selected from optionally substituted acyl, optionally substituted sulfonyl, optionally substituted alkyl, optionally substituted aralkyl, optionally substituted alkenyl, optionally substituted aryl, heteroaryl, or heterocyclyl.

According to a second aspect of the present invention there is provided a compound which is a multivalent presentation of the compounds of general formula (I) comprising a plurality of compounds of general formula (I) each bound through a linker to a multivalent template.

According to a third aspect of the present invention there is provided a pharmaceutical composition comprising a compound of general formula (I) and a pharmaceutically acceptable carrier.

According to a fourth aspect of the present invention there is provided a method of preventing or treating influenza in a subject comprising administering to said subject a compound of general formula (I).

According to a fifth aspect of the present invention there is provided the use of a compound of general formula (I) in the manufacture of a′ medicament for the prevention or treatment of influenza.

According to a sixth aspect of the present invention there is provided the use of a compound of general formula (I) in the prevention or treatment of influenza.

According to a seventh aspect of the present invention there is provided a method of preparing a compound of general formula (I), comprising the steps of:
1) providing a compound of general formula (IV), wherein:
   X₂, X₃/X₄ and Xₛ are as defined and may be protected by'protecting groups,
   X₆ is X₁, or a functional group that can be modified to form X₁, where X₆ can be selected from, but is not limited to, CHO, CN, CH₂OR′, thiazole, and
   Z is a group that can be activated to enable beta-elimination;

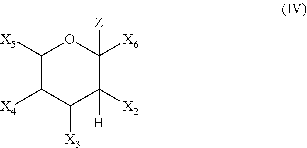

2) eliminating H—Z from the compound of general formula (IV);
3) converting X₆ to X₁ when it is other than X₁;
4) optionally functionalizing X₁, X₂, X₃, X₄ and/or X₅; and
5) optionally deprotecting X₁, X₂, X₃, X₄ and/or X₅.
   In an embodiment:

Z is a halide and elimination is achieved under basic conditions; or

Z is a halide and elimination is achieved in the presence of a heavy metal reagent; or

Z is acyloxy and elimination is achieved under Lewis acidic conditions; or

Z is alkoxy and elimination is achieved under acetolysis conditions; or

Z is phosphite and elimination is achieved under Lewis acidic conditions.

A compound of general formula IV where Z is halide can be formed by halogenation of a compound of the general formula VI where Q can be selected from, but is not limited to, —COOR′, —CN, —CH₂OR′.

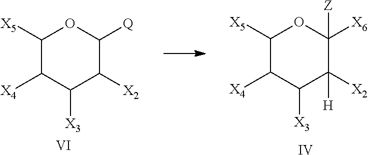

According to an eighth aspect of the present invention there is provided a method of preparing a compound of general formula (I), comprising the steps of:
1) providing a compound of general formula (V):, wherein X₂, X₃, X₄ and X₅ are as defined and may be protected by protecting groups;
2) introducing X₁ to the compound of general formula (V) in a direct C-1 lithiation followed by reaction of the lithiated species with EX₁ wherein E is an electrophile and X₁ may be protected with a protecting group;
3) optionally functionalizing X₁, X₂, X₃, X₄ and/or X₅; and
4) optionally deprotecting X₁, X₂, X₃, X₄ and/or X₅.

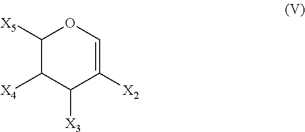

In an embodiment E is a halogen. Typically X₁ is protected with an alkyl group, which can be removed by hydrolysis.

### BRIEF DESCRIPTION OF THE DRAWINGS

FIG. 1. A. Superimposition of influenza A virus N8 sialidase-inhibitor complexes of 3-allyl-NeuAc2en (7) (dark grey; complex obtained after 60 min. soak) and Neu5Ac2en (white; PDB: 2 htr). B. N8-(7) complex with (7) shown in CPK format. C. N8-Neu5Ac2en complex (Russell et al., 2006) with C-3 unsubstituted Neu5Ac2en shown in CPK format. The 3-allyl-Neu5Ac2en complex maintains the ‘open’ conformation of the 150-loop seen in the apo structure (Russell et al., 2006), in contrast to the complex with Neu5Ac2en where the 150-loop is ‘closed’ (FIG. 1C).

FIG. 2. Influenza A virus N8 sialidase-inhibitor complex of 3-phenylallyl-Neu5Ac2en (9c). The N8-(9c) complex maintains an ‘open’ conformation of the 150-loop with the C-3 phenylallyl substituent extending into the 150-cavity.

FIG. 3. Influenza A virus N8 sialidase-inhibitor complex of 3-(p-tolyl)allyl-Neu5Ac2en (9d). Left panel: 3-(p-tolyl)allyl-Neu5Ac2en (9d) in stick format; Right panel: 3-(p-tolyl)allyl-Neu5Ac2en (9d) in CPK format. The N8-(9d) complex maintains an ‘open’ conformation of the 150-loop with the C-3 (p-tolyl)allyl substituent extending well into the 150-cavity.

FIG. 4. Superimposition of influenza A virus N8 X-ray crystal structures: open 150-loop N8/(9d) complex; closed 150-loop N8/Neu5Ac2en complex (PDS: 2 htr) (ligands in stick format), showing position of Asp-151. The dihydropyran ring and C-2, C-4, C-5, and C-6 substituents of (9d) and Neu5Ac2en have very similar positions in the active site. The phenyl ring of (9d) lies adjacent to Asp-151 in the open-loop conformation.

### DETAILED DESCRIPTION OF PREFERRED EMBODIMENTS

The invention discloses compounds that selectively inhibit influenza A virus group 1 sialidases and may therefore interrupt the infectious cycle of influenza A virus strains. In particular the invention is concerned with compounds of general formula (I);

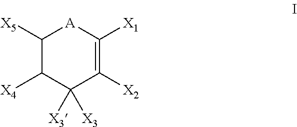

or a pharmaceutically acceptable salt, ester or prodrug thereof, wherein

A is O, S or NR₁;

where R₁ is hydrogen, optionally substituted alkyl, optionally substituted aryl, optionally substituted acyl or optionally substituted sulfonyl;

X₁ is CO₂H, P(O)(OH)₂, NO₂, SO₂H, SO₃H, —C(O)NHOH or tetrazoles;

X₂ is alkyl, aralkyl, alkenyl, alkynyl, optionally substituted alkyl, optionally substituted aralkyl, optionally substituted alkenyl, optionally substituted alkynyl, OR₂, SR₂, NR₂R₂′, or substituted triazole,

where R₃ and R₂′ are selected independently from optionally substituted acyl, optionally substituted sulfonyl, alkyl, alkenyl, alkynyl, optionally substituted alkyl, or optionally substituted alkenyl,

or R₂′ is hydrogen;

X₃ and X₃′ are selected independently from hydrogen, R₃, halogen, CN, OR₃, NR₃R₃′, NHC(NR₃)N(R₃)₂, N₃, SR₃, —O—CH₂—C(O)—NR₃R₃′, —O—CH₂—C(NH)—NR₃R₃′, —O—CH₂—C(S)—NR₃R₃′

and optionally substituted triazole,

or X₃ and X₃′ together are ═O, ═N—OR₃, or ═CH—R₃

where R₃ and R₃′ are selected independently from hydrogen, optionally substituted acyl, optionally substituted sulfonyl, alkyl, aralkyl, alkenyl, alkynyl, heteroalkyl, heterocyclyl, optionally substituted alkyl, optionally substituted aralkyl, optionally substituted alkenyl, —C(O)R₈ and —S(O)₂R₈,

where R₈ is selected from optionally substituted alkyl and optionally substituted alkenyl;

X₄ is NR₄ R₄′, O R₄, S R₄, CH₂C (O) R₄, CH₂C(O)OR₄, CH₂C(O)N R₄ R₄′, CH R₄NO₂, CH R₄CN, CH R₄ R₄′, or CH₂NHR,

where R₄ and R₄′ are selected independently from hydrogen optionally substituted acyl, optionally substituted thioacyl, optionally substituted sulfonyl, alkyl, alkenyl, alkynyl, optionally substituted alkyl, optionally substituted aralkyl, optionally substituted alkenyl, optionally substituted heteroaryl, and optionally substituted heterocyclyl;

X₅ is optionally substituted alkyl, optionally substituted aralkyl, optionally substituted alkenyl, optionally substituted alkynyl, optionally substituted heteroaryl, optionally substituted heterocyclyl, —C(O)R₅, —CO₂R₅, —C(O)NR₅R₅′, —P(O)(OR₅)(OR₅′), —P(O)(OR₅)(NR₅R₅′), —P(O)(NR₅R₅′)₂, CN, OR₆, azide, NHR₆, NR₆R₆′, SR₆, or optionally substituted triazole,

where R₅ and R₅′ are independently selected from hydrogen, optionally substituted alkyl, optionally substituted alkenyl, optionally substituted aryl, or heteroaryl, and

R₆ and R₆′ are independently selected from optionally substituted acyl, optionally substituted sulfonyl, optionally substituted alkyl, optionally substituted aralkyl, optionally substituted alkenyl, optionally substituted aryl, heteroaryl, or heterocyclyl.

In an embodiment X₅ denotes CH₂YR₇, CHYR₇CH₂YR₇ or CHYR₇CHY R₇CH₂YR₇,

where Y is O, S, or NR₇′, and successive Y moieties in an X₅ group are the same or different, or

where the substituent YR₇ is ═O, ═N—OR₇, or ═CHR₇, or

where two adjacent YR₇ groups together form part of a ring structure which optionally includes at least one heteroatom selected from O, S and N and is optionally substituted; in particular, an epoxide, aziridine, 5 or 6 membered cyclic ether group,

and R₇ and R₇′ are independently selected from hydrogen, optionally substituted acyl, optionally substituted sulfonyl, —S(O)₂OH, —P(O)(OH)₂, optionally substituted alkyl, optionally substituted aralkyl, optionally substituted alkenyl, optionally substituted aryl, heteroaryl, or heterocyclyl.

In an embodiment the compounds are of general formula (II), with the stereochemistry as shown;

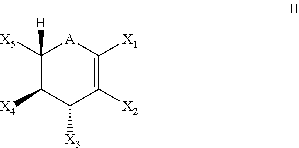

In an embodiment A is O.

In an embodiment X₁ is CO₂H or P(O)(OH)₂ or an ester thereof. The ester will readily hydrolyse in vivo into the free acid. In an embodiment X₁ is CO₂H.

In an embodiment X₃′ is H and X₃ is selected from R₃, halogen, CN, OR₃, NR₃R₃′, NHC(NR₃)N(R₃)₂, N₃, SR₃ and optionally substituted triazole,

where R₃ and R₃′ are independently selected from alkyl, alkenyl, alkynyl, optionally substituted alkyl, optionally substituted alkenyl, —C(O)R₈ or —S(O)₂R₈,

where R₈ is selected from optionally substituted alkyl and optionally substituted alkenyl.

In an embodiment X₄ is —NR₄R₄′. Advantageously R₅ is optionally substituted acyl and R₅′ is hydrogen, typically acyl such as acetyl.

In an embodiment the compounds are of general formula (III), with the stereochemistry as shown;

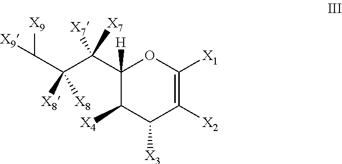

wherein X₁, X₂, X₃, and X₄ are as described above,

one of X₇ and X₇′ is hydrogen,

one of X₈ and X₈′ is hydrogen,

one of X₉ and X₉′ is hydrogen, and

X₇, X₇′, X₈, X₈′, X₉, and X₉′ are the same or different, and are selected from H, OR₇, NR₇R₇′, SR₇, or optionally substituted triazole, or

together X₇ and X₇′, X₈ and X₈′, or X₉ and X₉′ form ═O, or ═N—OR₇.

In an embodiment the compounds are selected from the group consisting of:
methyl 5-acetamido-4,7,8,9-tetra-O-acetyl-2,6-anhydro-3,5-dideoxy-3-C-(prop-2′-enyl)-D-glycero-D-galacto-non-2-enonate,
5-acetamido-2,6-anhydro-3,5-dideoxy-3-C-(prop-2′-enyl)-D-glycero-D-galacto-non-2-en-onic acid,
methyl 5-acetamido-4,7,8,9-tetra-O-acetyl-2,6-anhydro-3,5-dideoxy-3-C-(4,4-dimethylpent-2′-enyl)-D-glycero-D-galacto-non-2-enonate,
5-acetamido-2,6-anhydro-3,5-dideoxy-3-O-(4,4-dimethylpent-2′-enyl)-D-glycero-D-galacto-non-2-enonic acid,
methyl 5-acetamido-4,7,8,9-tetra-O-acetyl-2,6-anhydro-3,S-dideoxy-3-C-(3′-cyclohexyl-prop-2′-enyl)-D-glycero-D-galacto-non-2-enonate,
5-acetamido-2,6-anhydro-3,5-dideoxy-3-C-(3′-cyclohexyl-prop-2′-enyl)-D-glycero-D-galacto-non-2-enonic acid,
methyl 5-acetamido-4,7,8,9-tetra-O-acetyl-2,6-anhydro-3,5-dideoxy-3-C-(3′-phenyl-prop-2′-enyl)-D-glycero-D-galacto-non-2-enonate,
5-acetamido-2,6-anhydro-3,5-dideoxy-3-C-(3-phenyl-prop-2′-enyl)-D-glycero-D-galacto-non-2-enonic acid,
methyl 5-acetamido-4,7,8,9-tetra-O-acetyl-2,6-anhydro-3,5-dideoxy-3-C-[3′-(p-tolyl)-prop-2′-enyl]-D-glycero-D-galacto-non-2-enonate (8d, R=4-CH₃Ph),
5-acetamido-2,6-anhydro-3,5-dideoxy-3-C-[3′-(p-tolyl)-prop-2′-enyl]-D-glycero-D-galacto-non-2-enonic acid,
methyl 5-acetamido-4,7,8,9-tetra-O-acetyl-2,6-anhydro-3,5-dideoxy-3-C-[3′-(4-tert-butoxyphenyl)-prop-2′-enyl]-D-glycero-D-galacto-non-2-enonate,
5-acetamido-2,6-anhydro-3,5-dideoxy-3-C-[3′-(4-tert-butoxyphenyl)-prop-2′-enyl]-D-glycero-D-galacto-non-2-enonanic acid,
methyl 5-acetamido-4,7,8,9-tetra-O-acetyl-2,6-anhydro-3,5-dideoxy-3-C-(3′-naphthyl-prop-2′-enyl)-D-glycero-D-galacto-non-2-enonate,
5-acetamido-2,6-anhydro-3,5-dideoxy-3-C-(3′-naphthyl-prop-2′-enyl)-D-glycero-D-galacto-non-2-enonic acid,
methyl 5-acetamido-4,7,8,9-tetra-O-acetyl-2,6-anhydro-3,5-dideoxy-3-C-[4′-(3,4-dimethoxyphenyl)-but-2′-enyl]-D-glycero-D-galacto-non-2-enonate,
5-acetamido-2,6-anhydro-3,5-dideoxy-3-C-[4′-(3,4-dimethoxyphenyl)-but-2′-enyl]-D-glycero-D-galacto-non-2-enonic acid,
methyl 5-acetamido-3-C-(3′-acetoxypropyl)-4,7,8,9-tetra-O-acetyl-2,6-anhydro-3,5-dideoxy-D-glycero-D-galacto-non-2-en-onate,
5-acetamido-3-C-(3′-hydroxypropyl)-2,6-anhydro-3,5-dideoxy-D-glycero-D-galacto-non-2-enonic acid,
methyl 5-acetamido-4,7,8,9-tetra-O-acetyl-2,6-anhydro-3,5-dideoxy-3-C-propyl-D-glycero-D-galacto-non-2-enonate,
5-acetamido-2,6-anhydro-3,5-dideoxy-3-C-propyl-D-glycero-D-galacto-non-2-enonic acid,
methyl 5-acetamido-4,7,8,9-tetra-O-acetyl-2,6-anhydro-3,5-dideoxy-3-C-propenyl-D-glycero-D-galacto-non-2-enonate,
methyl 5-acetamido-2,6-anhydro-3,5-dideoxy-3-C-(prop-2′-enyl)-D-glycero-D-galacto-non-2-enonate,
methyl 5-acetamido-2,6-anhydro-3,5-dideoxy-8,9-O-isopropylidene-3-C-(prop-2′-enyl)-D-glycero-D-galacto-non-2-enonate,
methyl 5-acetamido-2,6-anhydro-3,5-dideoxy-4-O-ethyl-8,9-O-isopropylidene-3-C-(prop-2′-enyl)-D-glycero-D-galacto-non-2-enonate,
methyl 5-acetamido-2,6-anhydro-3,5-dideoxy-4-O-ethyl-8,9-O-isopropylidene-3-C-(prop-2′-enyl)-D-glycero-D-galacto-non-2-enonate,
5-acetamido-2,6-anhydro-3,5-dideoxy-4-O-ethyl-3-C-(prop-2′-enyl)-D-glycero-D-galacto-non-2-enonic acid,
2-methyl-(methyl 7,8,9-tri-O-acetyl-2,6-anhydro-3,5-dideoxy-3-C-(prop-2′-enyl)-D-glycero-D-talo-non-2-enonate)-[4,5-d]-2-oxazoline,
methyl 5-acetamido-7,8,9-tri-O-acetyl-2,6-anhydro-4-azido-3-C-(prop-2′-enyl)-3,4,5-trideoxy-D-glycero-D-galacto-non-2-enonate,
methyl 5-acetamido-4,7,8,9-tetra-O-acetyl-2,6-anhydro-3,5-dideoxy-3-O-ethyl-D-glycero-D-galacto-non-2-enonate,
5-acetamido-2,6-anhydro-3,5-dideoxy-3-O-ethyl-D-glycero-D-galacto-non-2-enonic acid,
methyl 5-acetamido-4,7,8,9-tetra-O-acetyl-2,6-anhydro-3,5-dideoxy-3-O-(2′-azidoethyl)-D-glycero-D-galacto-non-2-enonate, and
methyl 5-acetamido-4,7,8,9-tetra-O-acetyl-2,6-anhydro-3,5-dideoxy-3-O-(2′-(4″-isobutyl-[1″,2″,3″]triazol-1n-yl)ethyl)-D-glycero-D-galacto-non-2-enonate.

It will be appreciated that the manner of representing substituents in the foregoing general formula does not imply any particular stereochemistry or orientation for the substituents unless that is specifically shown. In particular, where compounds are optically active both (R) and (S) enantiomers or a mixture of the two, including a racemic mixture, are envisaged unless otherwise specified.

The term “alkyl” used either alone or in a compound word such as “optionally substituted alkyl” or “optionally substituted cycloalkyl” denotes straight chain, branched or mono- or poly-cyclic alkyl. Examples of straight chain and branched C alkyl include methyl, ethyl, propyl, isopropyl, butyl, isobutyl, sec-butyl, tert-butyl, amyl, isoamyl, sec-amyl, 1,2-dimethylpropyl, 1,1-dimethylpropyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, nonyl, 1-, 2-, 3-, 4-, 5-, 6- or 7-methyloctyl, 1-, 2-, 3-, 4- or 5-ethylheptyl, 1-2- or 3-propylhexyl, decyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, undecyl 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1-2-pentylheptyl and the like. Examples of cycloalkyl include cyclopropyl, cyclobutyl, cyclopentyl, cyclohexyl, cycloheptyl, cyclooctyl, cyclononyl and cyclodecyl and the like. In an embodiment alkyl is C1-C5 alkyl.

The term “alkenyl” used either alone or in compound words such as “alkenyloxy” denotes groups formed from straight chain, branched or cyclic alkenes including ethylenically mono-, di- or poly-unsaturated alkyl or cycloalkyl groups as defined above. Examples of alkenyl include allyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1-4,pentadienyl, 1,3-cyclopentadienyl, 1,3-hexadienyl, 1,4-hexadienyl, 1,3-cyclohexadienyl, 1,4-cyclohexadienyl, cycloheptadienyl, 1,3,5-cycloheptatrienyl and 1,3,5,7-cyclooctatetraenyl. In an embodiment alkenyl is C2-C5 alkenyl.

The term “acyl” used either alone or in compound words such as “optionally substituted acyl” denotes an aliphatic acyl group or an acyl group containing an aromatic ring, which is referred to as aromatic acyl, or a heterocyclic ring, which is referred to as heterocyclic acyl, but also includes such groups when oxygen is replaced with sulphur or an N═H group, and further includes such groups containing either one or two additional heteroatoms bonded to —C(O), —C(S) or —C(N═H). According, the term acyl, envisages —C(O)—, —C(S)—, —C(NH)—, —O—C(O)—, —O—C(S)—, —O—C(N═H)—, —S—C(O)—, —S—C(S)—, —S—C(N═H)—, —NH—C(O)—, —NH—C(S)—, —NH—C(N═H), —O—C(O)—O—, —O—C(S)—O—, —O—C(N═H)—O—, —S—C(S)—S—, —NH—C(N═H)—NH—, and son on. In embodiments an acyl group may include between 1 and 30 carbon atoms but more commonly is an aliphatic C1-C5 acyl such as acetyl. Examples of acyl include straight chain or branched alkanoyl such as formyl, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, pentadecanoyl, hexadecanoyl, heptadecanoyl, octadecanoyl, nonadecanoyl and icosanoyl; cycloalkylcarbonyl such as cyclopropylcarbonyl cyclobutylcarbonyl, cyclopentylcarbonyl and cyclohexylcarbonyl; aroyl such as benzoyl, toluoyl and naphthoyl; aralkanoyl such as phenylalkanoyl (e.g. phenylacetyl, phenylpropanoyl, phenylbutanoyl, phenylisobutyl, phenylpentanoyl and phenylhexanoyl) and naphthylalkanoyl (e.g. naphthylacetyl, naphthylpropanoyl and naphthylbutanoyl); aralkenoyl such as phenylalkenoyl (e.g. phenylpropenoyl, phenylbutenoyl, phenylmethacrylyl, phenylpentenoyl and phenylhexenoyl and naphthylalkenoyl (e.g. naphthylpropenoyl, naphthylbutenoyl and naphthylpentenoyl); heterocycliccarbonyl; heterocyclicalkanoyl such as thienylacetyl, thienylpropanoyl, thienylbutanoyl, thienylpentanoyl, thienylhexanoyl, thiazolylacetyl, thiadiazolylacetyl and tetrazolylacetyl; and heterocyclicalkanoyl such as heterocyclicpropenoyl, heterocyclicbutenoyl, haterocyclicpentenoyl and heterocyclichexenoyl.

The term “sulfonyl” used either alone or in compound words such as “optionally substituted sulfonyl” denotes one of the groups —S(O)₂R₉ wherein each R₉ is independently H, optionally substituted alkyl or optionally substituted aryl. Accordingly the group in its entirety may be, for example, a sulfonate ester or amide, depending on the context, such as —O—S(O)₂R₉ or —NR₄—S—(O)₂R₉.

The term “aryl” used either alone or in compound words such as “optionally substituted aryl”, “optionally substituted aryloxy” or “optionally substituted heteroaryl” denotes single, polynuclear, conjugated and fused residues of aromatic hydrocarbons (“carbocyclic aryl” or “carboaryl”) or aromatic heterocyclic (“heteroaryl”) ring systems. Examples of carbocyclic aryl include phenyl, biphenyl, terphenyl, quaterphenyl, phenoxyphenyl, napthyl, tetrahydronaphthyl, anthracenyl, dihydroanthracenyl, benzanthracenyl, dibenzanthracenyl, phenanthrenyl, fluorenyl, pyrenyl, indenyl, azulenyl, chrysenyl. Examples of heteroaryl include pyridyl, 4-phenylpyridyl, 3-phenylpyridyl, thienyl, furyl, pyrryl, pyrrolyl, furanyl, imadazolyl, pyrrolydinyl, pyridinyl, piperidinyl, indolyl, pyridazinyl, pyrazolyl, pyrazinyl, thiazolyl, pyrimidinyl, quinolinyl, isoquinolinyl, benzofuranyl, benzothienyl, purinyl, quinazolinyl, phenazinyl, acridinyl, benzoxazolyl, benzothiazolyl and the like. Preferably, a carbocyclic aromatic ring system contains 6-10 carbon atoms and an aromatic heterocyclic ring system contains 1 to 4 heteratoms independently selected from N, O and S and up to 9 carbon atoms in the ring.

The term “heterocyclyl” or equivalent terms such as “heterocyclic” used either alone or in compound words such as “optionally substituted saturated or unsaturated heterocyclyl” denotes monocyclic or polycyclic heterocyclyl groups containing at least one heteroatom atom selected from nitrogen, sulphur and oxygen. Suitable heterocyclyl groups include N-containing heterocyclic groups, such as, unsaturated 3 to 6 membered heteromonocyclic groups containing 1 to 4 nitrogen atoms, for example, pyrrolyl, pyrrolinyl, imidazolyl, pyrazolyl, pyridyl, pyrimidinyl, pyrazinyl, pyridazinyl, triazolyl or tetrazolyl;

saturated 3 to 6-membered heteromonocyclic groups containing 1 to 4 nitrogen atoms, such as, pyrrolidinyl, imidazolidinyl, piperidino or piperazinyl;

unsaturated condensed heterocyclic groups containing 1 to 5 nitrogen atoms, such as indolyl, isoindolyl, indolizinyl, benzimidazolyl, quinolyl, isoquinolyl, indazolyl, benzotriazolyl or tetrazolopyridazinyl;

unsaturated 3 to 6-membered heteromonocyclic group containing an oxygen atom, such as oxiranyl, pyranyl or furyl:

unsaturated 3 to 6-membered heteromonocyclic group containing 1 to 2 sulfur atoms, such as, thienyl;

unsaturated 3 to 6-membered heteromonocyclic group containing 1 to 2 oxygen atoms and 1 to 3 nitrogen atoms, such as, oxazolyl, isoxazolyl or oxadiazolyl;

saturated 3 to 6-membered heteromonocyclic group containing 1 to 2 oxygen atoms and 1 to 3 nitrogen atoms, such as, morpholinyl;

unsaturated condensed heterocyclic group containing 1 to 2 oxygen atoms and 1 to 3 nitrogen atoms, such as, benzoxazolyl or benzoxadiazolyl;

unsaturated 3 to 6-membered heteromonocyclic group containing 1 to 2 sulphur atoms and 1 to 3 nitrogen atoms, such as, thiazolyl or thiadiazolyl;

saturated 3 to 6-membered heteromonocyclic group containing 1 to 2 sulphur atoms and 1 to 3 nitrogen atoms, such as, thiazolidinyl; and

unsaturated condensed heterocyclic group containing 1 to 2 sulphur atoms and 1 to 3 nitrogen atoms, such as, benzothiazolyl or benzothiadiazolyl.

The term “carbohydrate” denotes a carbohydrate residue or a functionalised or deoxygenated carbohydrate residue, and includes monosaccharides and oligosaccharides. A carbohydrate residue is an acyclic polyhydroxy-aldehyde or ketone, or one of their cyclic tautomers, and includes a compound resulting from reduction of the aldehyde or keto group such as alditols. Oxygen atoms may be replaced by hydrogen or bonds to a halogen, nitrogen, sulfur or carbon atoms, or carbon-oxygen bonds such as in ethers or esters may be introduced. Examples of carbohydrates include but are not limited to D-galactose, D-galactofuranose, N-acetyl-D-galactofuranose, D-galactopyranose, N-acetyl-D-galactopyranose, D-glucose, D-glucofuranose, N-acetyl-D-glucofuranose, D-glucopyranose and N-acetyl-D-glucopyranose, D-mannose, D-mannofuranose, D-mannopyranose, N-acetyl-D-mannopyranose, D-arabinofuranose, D-arabinopyranose, L-rhamnopyranose, D-ribose, D-fucose, N-acylneuraminic acid, 2-keto-3-deoxy-nonulosonic acid, 2-keto-3-deoxy-octulosonic acid, D-galacturonic acid, D-glucuronic acid, D-muramic acid, D-fructose, D-galactitol, D-glucitol, D-mannitol, D-lactitol, and their equivalents where oxygen atoms have been replaced in selected positions with hydrogen or bonds to halogen, nitrogen, sulfur or carbon, as well as oligosaccharides containing these moieties.

In this specification “optionally substituted” means that a group may or may not be further substituted with one or more functional groups such as alkyl, alkenyl, alkynyl, aryl, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, benzylamino, dibenzylamino, acyl, alkenylacyl, alkynylacyl, arylacyl, acylamino, diacylamino, acyloxy, alkylsulphonyloxy, arylsulphenyloxy, heterocyclyl, heterocycloxy, heterocyclamino, haloheterocyclyl, alkylsulphenyl, arylsulphenyl, carboalkoxy, carboaryloxy, mercapto, alkylthio, benzylthio, acylthio, phosphorus-containing groups and the like, and including groups such as oxo, ═S, ═N—, where appropriate, particularly as substituents in ring structures such as lactones, lactams and cyclic imides, provided that none of the substituents outlined above interferes with the formation or activity of the subject compound.

Any of the moieties whose length is defined in terms of the number of carbon atoms present may possess any number of carbon atoms within the specified range. Nevertheless, within this range certain species will be preferred due to factors such as availability and cost of precursors and ease of synthesis, as well as efficacy.

The compounds of the invention may be prepared by manipulation of carbohydrate structures to introduce the functional groups as described in the general formulae. An extensive array of methodologies has been developed to manipulate different positions on carbohydrate templates as disclosed, for example, in Ernst, Hart & Sinay, 2000; Chapleur, 1998; and Stick, 2001; the contents of which are incorporated herein by reference. In particular, methodologies to manipulate each position of the neuraminic acid template have been developed as disclosed for example in Zbiral 1992; von Itzstein and Thomson, 1997; Kiefel and von Itzstein, 2002; the contents of which are incorporated herein by reference.

A number of general methods for the preparation of the compounds of the invention where X₁ is C(O)OH are shown in the following scheme. In compounds containing an alpha, beta-unsaturated carboxylate, halohydrin formation (path A) can be achieved using N-bromosuccinimide, as described for example in Okamoto et al., 1987. Radical reaction of the bromohydrin can be employed to introduce a carbon-linked substituent X₂ using Bu₃Sn(X₂), as described for example in Paulsen and Matschulat, 1991. Chlorination or bromination at the alpha position and subsequent elimination of HX can be employed to give the beta-substituted alpha,beta-unsaturated derivative. Direct introduction of a carbon-linked substituent X₂ can be achieved through transition metal-mediated radical reaction with the alpha,beta-unsaturated carboxylate (path B). Radical addition to the double bond may be carried-out in the presence of a transition metal catalyst such as ceric(IV) ammonium nitrate or manganese triactetate, as described for example in Linker, 2002; Gyollai et al., 2002. Acetolysis of the alpha-methoxy group using sulfuric acid, acetic acid and acetic anhydride, such as described in Kok et al., 1999, can be employed to form the beta-substituted alpha,beta-unsaturated derivative.

In compounds derived from uronic acid derivatives such as disclosed in Florio et al., 1999; Smith et al., 1999; Florio, et al., 2000; Mann et al., 2006; the contents of which are incorporated herein by reference, bromination alpha to the carboxylate (path C), followed by elimination of HBr can be employed to form the beta-substituted alpha,beta-unsaturated derivative.

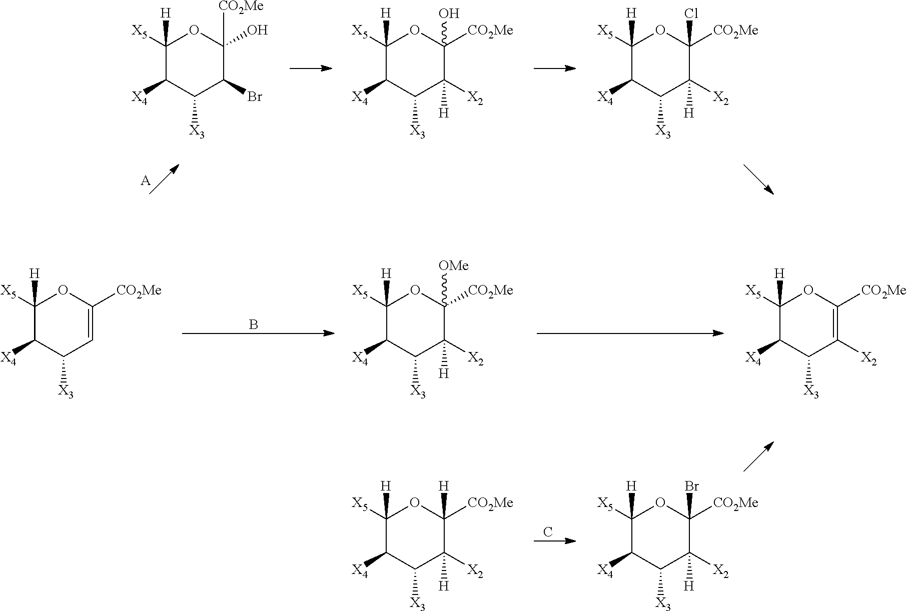

In an embodiment where X₂ is —CH₂CH═CH₂, further structural elaboration can be achieved through manipulation of the allyl group using a range of reactions including, but not limited to, hydrogenation, epoxidation [such as described for example in J. Am. Chem. Soc. (2003) 125, 924], halogenation [such as described for example in Chem. Rev. (1956), 56, 753-901], cycloaddition [such as described for example in J. Org. Chem. (2008), 73, 7164], addition of borane reagents (such as described in Falck-Pedersen et al., 2005), and olefin cross metathesis (such as described in Meinke and Thiem, 2008). Olefinic cross metathesis reactions can be performed using Ruthenium-based metathesis catalysts: Grubbs 1st generation (G-1), Hoveyda-Grubbs 1st generation (HG-1), Grubbs 2nd generation (G-2), Hoveyda-Grubbs 2nd generation (HG-2), and Grela's catalyst (Gre-2).

In an embodiment the multivalent array of the compounds comprises the following structure:

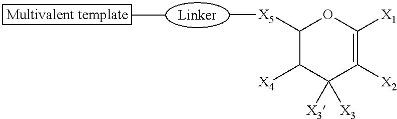

In an embodiment the multivalent template is selected from the group consisting of, but not limited to, polystyrene nanoparticles, ceramic nanoparticles, coated gold particles, di-, tri- and tetra-antennary structures and dendrimers (as described for example in Roy 1997), liposomes, micelles, and virus hybrid systems. Multivalent arrays of influenza virus sialidase inhibitors (principally zanamivir) are is described, by way of example, in WO 98/21243, WO 2000/055149 and WO 2002/020514, the contents of which are incorporated by reference.

The compounds of the invention interrupt the infectious cycle of influenza A virus strains, and therefore are useful in the prevention or treatment of influenza in a subject, particularly a human subject when administered in a therapeutically effective amount.

As used herein, the term “therapeutically effective amount” means an amount of a compound of the present invention effective to yield a desired therapeutic response, for example to prevent or treat a disease by administration of a pharmaceutically-active agent.

The specific “therapeutically effective amount” will, obviously, vary with such factors as the particular condition being treated, the physical condition and clinical history of the subject, the type of animal being treated, the duration of the treatment, the nature of concurrent therapy (if any), and the specific formulations employed and the structure of the compound or its derivatives.

As used herein, a “pharmaceutically acceptable carrier” is a pharmaceutically acceptable solvent, suspending agent, excipient or vehicle for delivering the compound of general formula (I) to the subject. The carrier may be liquid or solid, and is selected with the planned manner of administration in mind.

It will be appreciated that pharmaceutically acceptable derivatives of the compounds of general formula I and the salts thereof, are also within the scope and spirit of the invention. Such derivatives includes pharmaceutically acceptable esters, prodrugs, solvates and hydrates of the compounds or their salts. Pharmaceutically acceptable derivatives may include any solvate, hydrate or any other compound or prodrug which, upon administration to a subject, is capable of providing (directly or indirectly) a compound of formula I or an antivirally active metabolite or residue thereof.

The pharmaceutically acceptable salts include acid addition salts, base addition salts, salts of pharmaceutically acceptable esters and the salts of quaternary amines and pyridiniums. The acid addition salts are formed from a compound of the invention and a pharmaceutically acceptable inorganic or organic acid including but not limited to hydrochloric, hydrobromic, sulphuric, phosphoric, methanesulfonic, toluenesulphonic, benzenesulphonic, acetic, propionic, ascorbic, citric, malonic, fumaric, maleic, lactic, salicyclic, sulfamic, or tartartic acids. The counter ion of quaternary amines and pyridiniums include chloride, bromide, iodide, sulfate, phosphate, methansulfonate, citrate, acetate, malonate, fumarate, sulfamate, and tartate. The base addition salts include but are not limited to salts such as sodium, potassium, calcium, lithium, magnesium, ammonium and alkylammonium. Also, basis nitrogen-containing groups may be quaternised with such agents as lower alkyl halides, such as methyl, ethyl, propyl, and butyl chlorides, bromides and iodides; dialkyl sulfates like dimethyl and diethyl sulfate; and others. The salts may be made in a known manner, for example by treating the compound with an appropriate acid or base in the presence of a suitable solvent.

The compounds of the invention may be in crystalline form either as the free compounds or as solvates (e.g. hydrates) and it is intended that both forms are within the scope of the present invention. Methods of solvation are generally known in the art.

The term “solvate” is a complex of variable stoichiometry formed by a solute (in this invention, a compound of the invention) and a solvent. Such solvents preferably do not interfere with the biological activity of the solute. Solvents may be, by way of example, water, ethanol or acetic acid. Methods of salvation are generally known within the art.

The term “pro-drug” is used in its broadest sense and encompass those derivatives that are converted in vivo to the compounds of the invention. Such derivates would readily occur to those skilled in the art, and include, for example, compounds where a free hydroxyl group is converted into an ester derivative or a ring nitrogen atom is converted to an N-oxide. Examples of ester derivatives include alkyl esters, phosphate esters and those formed from amino acids, preferably valine. Any compound that is a prodrug of a compound of the invention is within the scope and spirit of the invention. Conventional procedures for the preparation of suitable prodrugs according to the invention are described in text books, such as “Design of Prodrugs” Ed. H. Bundgaard, Elsevier, 1985.

The compound of general formula (I) may be administered in any convenient form including orally, topically, or parenterally in dosage unit formulations containing conventional non-toxic pharmaceutically acceptable carriers, adjuvants, and vehicles. The compounds can be administered, for in vivo application, parenterally by injection or by gradual perfusion over time independently or together. Administration may be intravenously, intra-arterial, intraperitoneally, intramuscularly, subcutaneously, intracavity, transdermally or by inhalation. Inhalation may be by way a dry powder inhaler, a metered dose inhaler or nebulizer as described, for example, in WO99/16421, the contents of which are incorporated herein by reference. For in vitro studies the agents may be added or dissolved in an appropriate biologically acceptable buffer and added to a cell or tissue.

Generally, the terms “treating”, “treatment” and the like are used herein to mean affecting a subject, tissue or cell to obtain a desired pharmacological and/or physiological effect. The effect may be prophylactic in terms of completely or partially preventing infection, and/or may be therapeutic in terms of a partial or complete cure of an infection. “Treating” as used herein covers any treatment of, or prevention of infection in a vertebrate, a mammal, particularly a human, and, includes: preventing the infection from occurring in a subject that may have been exposed to an influenza virus, but has not yet been diagnosed as affected; inhibiting the infection, ie., arresting its development; or relieving or ameliorating the effects of the infection, ie., cause regression of the effects of the infection.

The pharmaceutical compositions of the invention comprise a pharmaceutically acceptable carrier designed to bring a compound of the invention into a form suitable for administration to a subject using carriers, excipients and additives or auxiliaries.

Frequently used carriers or auxiliaries include magnesium carbonate, titanium dioxide, lactose, trehalose, mannitol and other sugars, talc, milk protein, gelatin, starch, vitamins, cellulose and its derivatives such as hydroxypropylmethyl cellulose, polymers such as polyvinylpyrrolidone(PVP) and polyethylene glycols, animal and vegetable oils, solvents such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412, 1461-1487 (1975) and The National Formulary XIV., 14th ed. Washington: American Pharmaceutical Association (1975), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed.). When desired the formulations may be adapted to give sustained release of the active ingredient.

The pharmaceutical compositions are preferably prepared and administered in dosage units. Solid dosage units include tablets, capsules and suppositories. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions according to the invention may be administered locally or systemically in a therapeutically effective dose. Amounts effective for this use will, of course, depend on the severity of the microbial infection and the weight and general state of the subject. Typically, dosages used in vitro may provide useful guidance in the amounts useful for in situ administration of the pharmaceutical composition, and animal models may be used to determine effective dosages for treatment of the cytotoxic side effects. Various considerations are described, eg., in Langer, Science, 249: 1527, (1990). Formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, such as peanut oil, liquid paraffin or olive oil.

Aqueous suspensions normally contain the active materials in admixture with excipients suitable for the manufacture of aqueous suspension. Such excipients may be suspending agents such as sodium carboxymethyl cellulose, methyl cellulose, hydroxypropylmethylcellulose, sodium alginate, polyvinylpyrrolidone, gum tragacanth and gum acacia; dispersing or wetting agents, which may be (a) naturally occurring phosphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate.

The pharmaceutical compositions may be in the form of a sterile injectable aqueous or oleaginous suspension. This suspension may be formulated according to known methods using suitable dispersing or wetting agents and suspending agents such as those mentioned above. The sterile injectable preparation may also be a sterile injectable solution or suspension in a non-toxic parenterally-acceptable diluent or solvent, for example, as a solution in 1,3-butanediol. Among the acceptable vehicles and solvents which may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid find use in the preparation of injectables.

Compounds of the invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. Compounds of general formula (I) may also be administered in combination with cyclodextrins for enhanced aqueous solubility.

The compounds of the invention may be administered by any of the methods and formulations employed in the art for intranasal administration. Thus in general the compounds may be administered in the form of a solution or a suspension or as a dry powder.

Solutions and suspensions will generally be aqueous, for example prepared from water alone (for example sterile or pyrogen-free water), or water and a physiologically acceptable co-solvent (for example ethanol, propylene glycol, and polyethylene glycols such as PEG 400). Such solutions or suspensions may additionally contain other excipients for example preservatives (such as benzalkonium chloride), solubilising agents/surfactants such as polysorbates (e.g. Tween 80, Span 80, benzalkonium chloride), buffering agents, isotonicity-adjusting agents (for example sodium chloride), absorption enhancers and viscosity enhancers. Suspensions may additionally contain suspending agents (for example microcrystalline cellulose, carboxymethyl cellulose sodium).

Solutions or suspensions are applied directly to the nasal cavity by conventional means, for example with a dropper, pipette or spray, or metered dose inhaler. The formulations may be provided in single or multi-dose fashion. In the latter case a means of dose metering is desirably provided. In the case of a dropper or pipette this may be achieved by the patient administering an appropriate, predetermined volume of the solution or suspension. In the case of a spray this may be achieved for example by means of a metering atomising spray pump.

Intranasal administration may also be achieved by means of an aerosol formulation in which the compound is provided in a pressurised pack with a suitable propellant such as a chlorofluorocarbon (CFC), for example dichlorodifluoromethane, trichlorofluoromethane or dichlorotetrafluororoethane, carbon dioxide or other suitable gas. The aerosol may conveniently also contain a surfactant such as lecithin. The dose of drug may be controlled by provision of a metered valve.

Alternatively the compounds may be provided in the form of a dry powder, for example a powder mix of the compound in a suitable powder base such as lactose, starch, starch derivatives such as hydroxypropylmethyl cellulose and polyvinylpyrrolidine (PVP). In an embodiment the powder carrier will form a gel in the nasal cavity. The powder composition may be presented in unit dose form, for example in capsules or cartridges of e.g. gelatin or blister packs from which the powder may be administered by means of an inhaler.

In the intranasal formulations the compound will generally have a small particle size, for example of the order of 5 microns or less. Such a particle size may be obtained by means known in the art, for example by micronisation.

Dosage levels of the compound of general formula (I) of the present invention will usually be of the order of about 0.05 mg to about 20 mg per kilogram body weight, with a preferred dosage range between about 0.05 mg to about 10 mg per kilogram body weight per day (from about 0.1 g to about 3 g per patient per day). The amount of active ingredient which may be combined with the carrier materials to produce a single dosage will vary, depending upon the host to be treated and the particular mode of administration. For example, a formulation intended for oral administration to humans may contain about 1 mg to 1 g of an active compound with an appropriate and convenient amount of carrier material, which may vary from about 5 to 95 percent of the total composition. Dosage unit forms will generally contain between from about 5 mg to 500 mg of active ingredient.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

The compounds of the invention may additionally be combined with other compounds to provide an operative combination. It is intended to include any chemically compatible combination of pharmaceutically-active agents, as long as the combination does not eliminate the activity of the compound of general formula (I) of this invention. In an embodiment are used in combination with other therapeutic agents, for example other anti-infective agents. In particular the compounds of the invention may be employed with other antiviral agents.

The invention thus provides in a further aspect a combination comprising a compound of formula (I) or a pharmaceutically acceptable salt or derivative thereof together with another therapeutically active agent, in particular an antiviral agent.

The combinations referred to above may conveniently be presented for use in the form of a pharmaceutical formulation and thus such formulations comprising a combination as defined above together with a pharmaceutically acceptable carrier therefore comprise a further aspect of the invention.

Suitable therapeutic agents for use in such combinations include other anti-infective agents, in particular anti-bacterial and anti-viral agents such as those used to treat respiratory infections. For example, other compounds effective against influenza viruses, such as amantadine, rimantadine and ribavirin, may be included in such combinations.

The individual components of such combinations may be administered either sequentially or simultaneously in separate or combined pharmaceutical formulations.

When the compounds of the invention are used with a second therapeutic agent active against the same virus, the dose of each compound may either be the same as or differ from that employed when each compound is used alone. Appropriate doses will be readily appreciated by those skilled in the art.

### MODES FOR PERFORMING THE INVENTION

Examples of synthetic schemes that can be employed to prepare compounds in accordance with preferred embodiments of the invention are now described in more detail. The methods described are intended to illustrate the nature of such preparations and are not intended to limit, the scope of the invention or of the applicable methods. Detailed description of the methods is found in the Experimental section below.

An exemplary method of preparing the compounds of the invention, where X₂ is linked through carbon to the scaffold, is shown in Scheme 1 below (described in Examples 1-4).

Introduction of a carbon-linked substituent can be performed by radical reaction on an alkyl bromide. In compounds containing an alpha,beta-unsaturated carboxylate, halide can be introduced beta to the carboxylate through halohydrin formation, for example using N-bromosuccinimide, as described for example in Okamoto et al., 1987. Radical reaction of the bromohydrin can be employed to introduce a carbon-linked substituent X₂ using Bu₃Sn(X₃), as described for example in Paulsen and Matschulat, 1991 (described in Example 1). The hydroxyl group alpha to the carboxylate is then converted to a leaving group suitable to enable beta-elimination. Methods of beta-elimination include activation of the position alpha to the carboxylate of the beta-substituted ester with halogen, phosphite [as described for example in Stolz, F. et al., J. Org. Chem. (2004) 69, 665-679] or acetate, and subsequent beta-elimination; for an alpha-halide under basic conditions [as described for example in Blattner (1980); Rye (2002)] (described in Examples 3, 22 and 24) or for an acetate or phosphite [as described for example in Stolz (2004)] under Lewis acidic conditions.

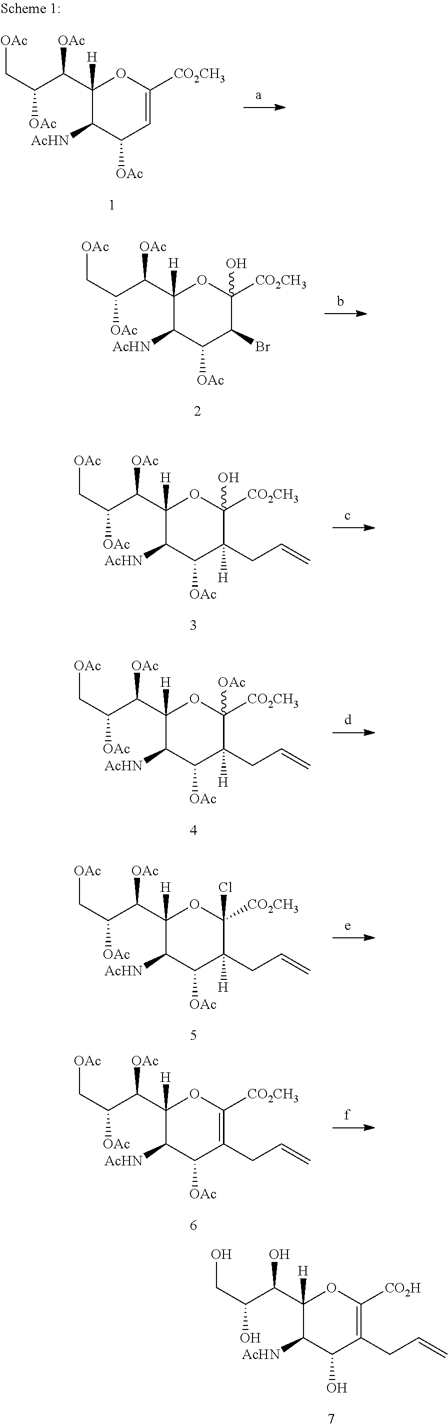

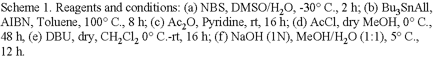

Exemplary methods for varying the substituent X₂, are shown in Schemes 2 to 5 (described in Examples 5-24). In an embodiment where X₂ is —CH₂CH═CH₂, further manipulation of the allyl group can be achieved using a range of reagents, for example using Grubbs catalyst as exemplified in Scheme 2 (described in Examples 5-18), and borane reagents as exemplified in Scheme 3 (described in Examples 19 and 20).

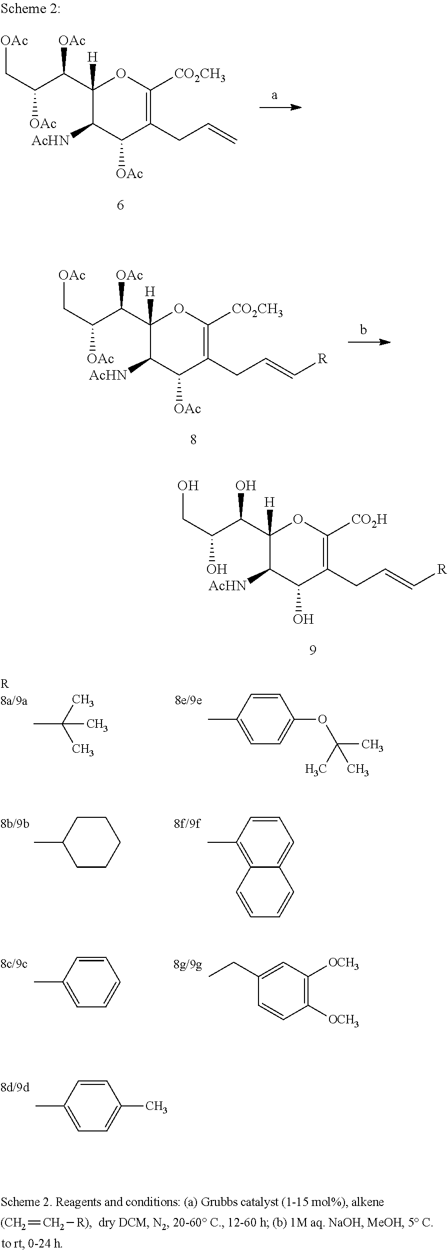

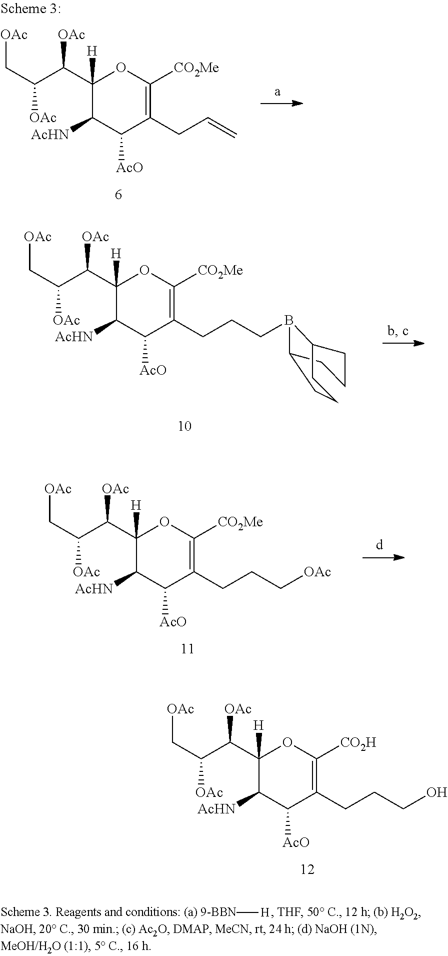

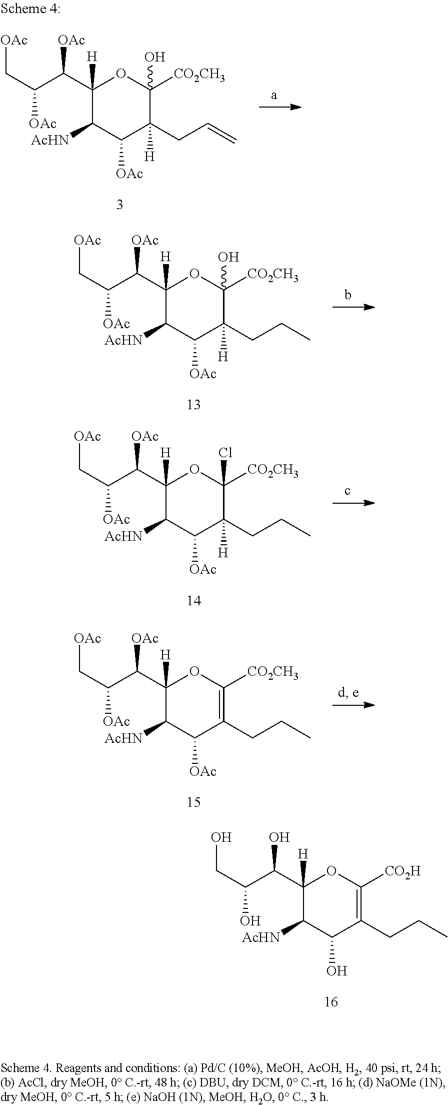

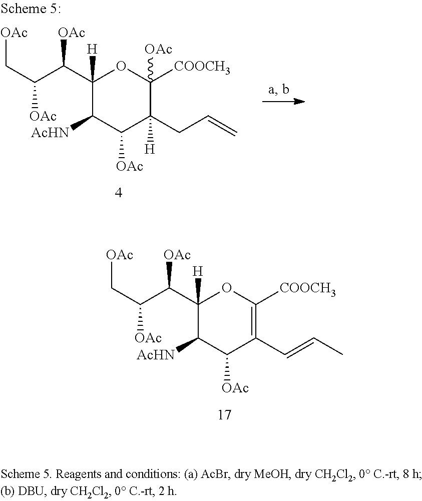

Exemplary methods for varying the substituent X₃ are shown in Schemes 6 to 8 (described in Examples 25-30).

Scheme 6: selective alkylation of the C-4 hydroxyl group of a suitably protected precursor can be achieved using an alkyl halide in the presence of Ag₂O or a hydride reagent (as exemplified in Scheme 6) [as described for example in Tindal, D. J. et al., Bioorg. Med. Chem. Lett. (2007) 17, 1655-1658; Ikeda, K. et al., Carbohydr. Res. (2001) 330, 31-41] (described in Examples 25-28). The introduced alkyl group can be further modified [as described for example in Ikeda, K. et al., Carbohydr. Res. (2001) 330, 31-41].

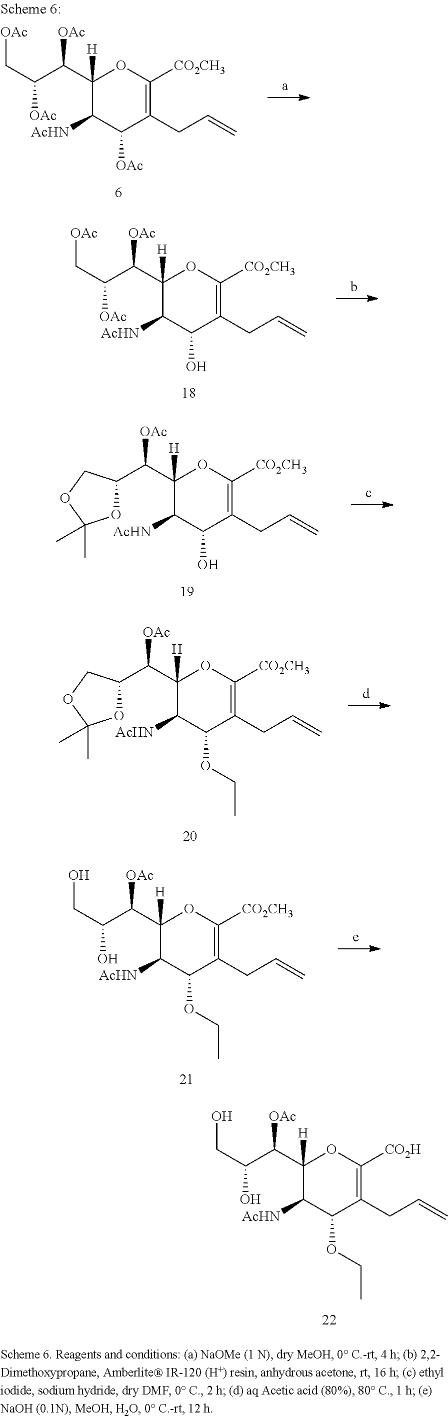

Schemes 7 and 8: formation of an oxazoline between the C-5 acetamide and the C-4 position (as exemplified in Schemes 7 and 8) allows subsequent introduction of a substituent (X₃) such as azide (as exemplified in Scheme 8) or thiolacetate at C-4 [as described for example in von Itzstein, M. at al., Carbohydr. Res. (1993) 244, 181-185] (described in Examples 29 and 30). The introduced azide group can be further modified [as described for example in: Chandler, M. et al. J. Chem. Soc. Perkin Trans. I (1995) 1173-1180; Lu and Gervay-Hague, Carbohydr. Res. (2007) 342, 1636-1650].

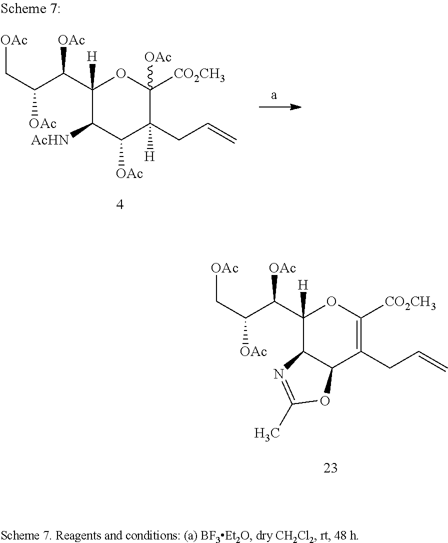

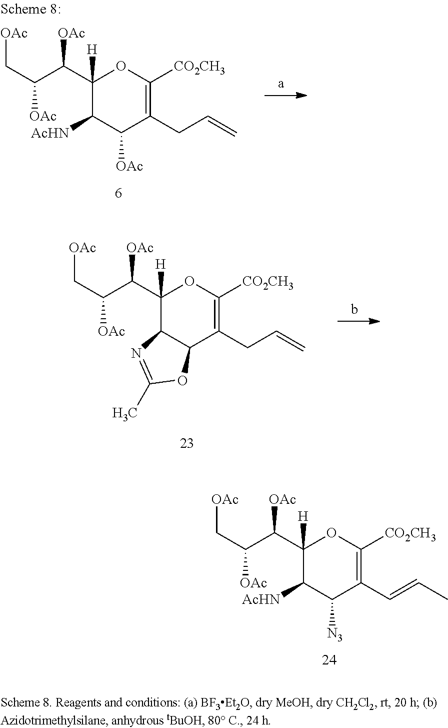

Exemplary methods of preparing the compounds of the invention, where X₂ is linked through oxygen to the scaffold, are shown in Schemes 9 and 10 (described in Examples 31-43).

Scheme 9: a hydroxyl group can be introduced beta to a carboxylate by manipulation of an alpha-beta unsaturated ester functionality (as exemplified in Scheme 9) through reaction of the alpha-beta unsaturated ester with a dihalide [as described for example in Okamoto, K. et al., Bull. Chem. Soc. Jpn. (1987) 60, 631-636] (Example 31), selective hydrolysis of the alpha bromide of the so-formed dibromide (for example as described in Example 31), formation of an epoxide from the so-formed bromohydrin [as described for example in Okamoto et al. (1987)] (described in Example 32), and ring-opening of the epoxide by attack at the position alpha to the carboxylate [as described for example in Okamoto et al. (1987)] (described in Example 33). The epoxide may be opened to introduce an alkyl group [as described for example in Okamoto et al. (1987)] or an acyl group [using a method such as described for example in Timmers, C. M. et al., J. Carbohydr. Chem. (1998) 17, 471-487]. The beta-hydroxyl group can be alkylated using an alkyl halide in the presence of Ag₂O or a hydride reagent (described in Examples 34 and 39). The substituent alpha to the carboxylate is then converted to a leaving group suitable to enable beta-elimination. When this substituent is p-methoxybenzyloxy, the p-methoxybenzyl group can be removed for example by oxidative cleavage with ceric ammonium nitrate (CAN) or 2,6-dichloro-5,6-dicyanobenzoquinone (DDQ) (described in Examples 35 and 40). Conversion of the alpha hydroxyl group to a leaving group can be performed as described above (Scheme 1). Introduction of bromine alpha to the carboxylate can be performed for example through conversion of the hydroxyl group to an acetate and subsequent reaction with a brominating reagent such as HBr/AcOH (described in Examples 37) or TMSBr (described in Examples 43). Beta-elimination of HBr to form the beta-substituted alpha-beta-carboxylate functionality can be performed using for example a base such DBU or triethylamine (such as described in Examples 37 and 43).

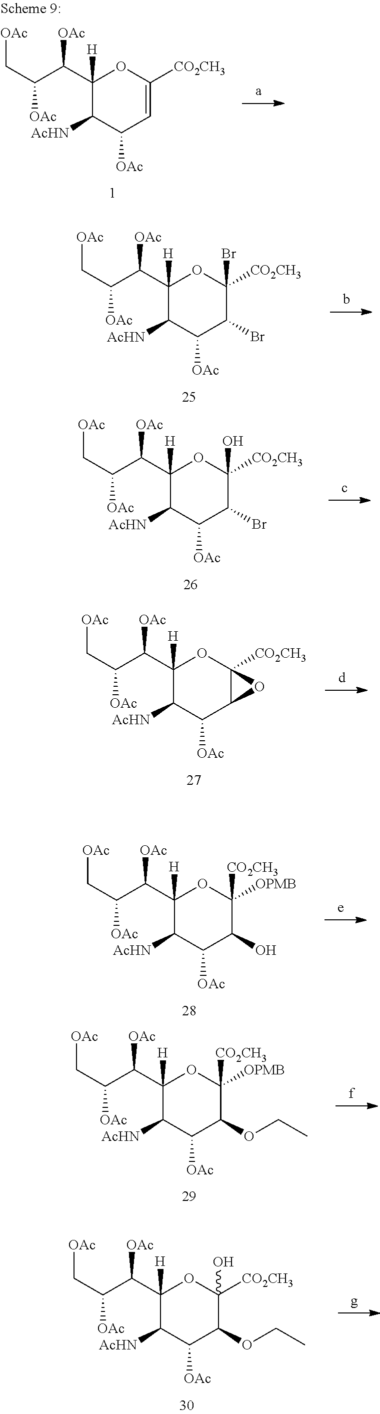

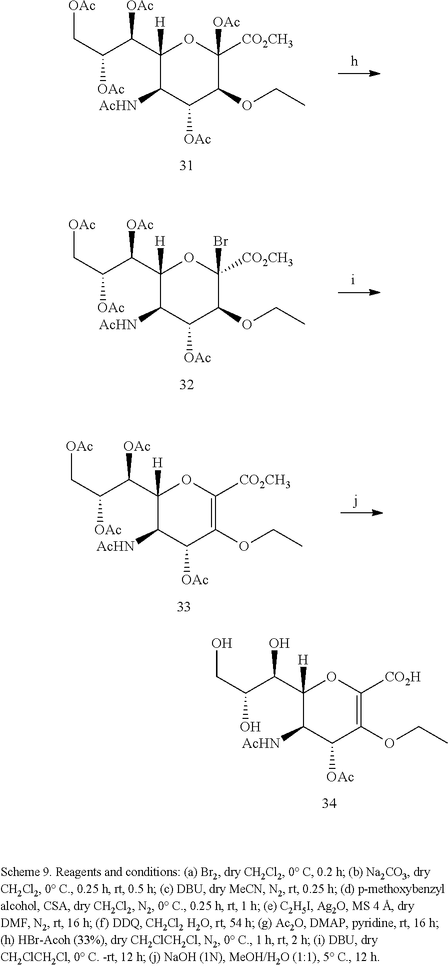

Exemplary methods for varying the substituent X₂, are shown in Schemes 10 and 11.

Scheme 10: the side chain introduced at C-3 can be further modified according to known procedures. For example, where X₂ is —O—CH₂CN, further manipulation of the cyano group can be achieved, for example, through reduction to the amine (described in Example 42), and subsequent conversion of the amine to an azide (described in Example 42). Where X₂ is —O—CH₂CH₂NH₂ [for example (38)] the amine can be further modified by acylation under standard conditions.

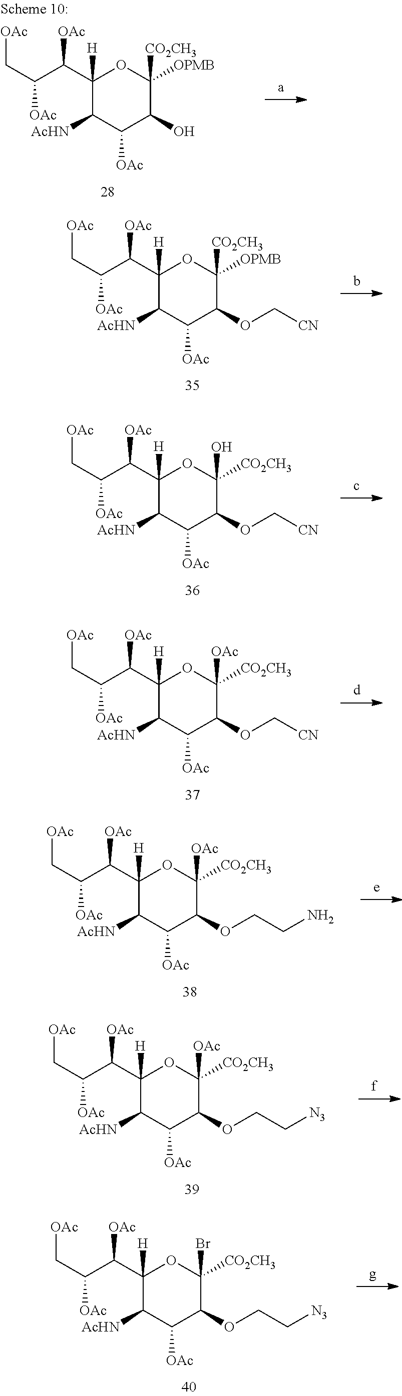

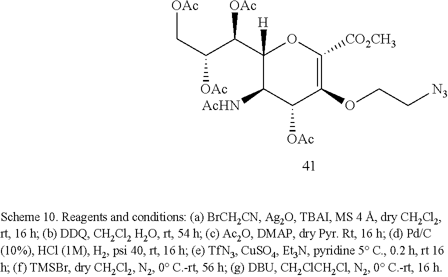

Scheme 11: an exemplary method for manipulation of the side-chain X₂ through elaboration of an azido group to a substituted triazole is shown in Scheme 11 (described in Examples 44 and 45). In a 1,3-dipolar cycloaddition reaction an azide can be reacted with a substituted alkyne to produce a substituted triazole [as described for example in Lu and Gervay-Hague, Carbohydr. Res. (2007) 342, 1636-1650; and reviewed in Bock, V. D. et al., Eur. J. Org. Chem. (2006) 51-68.].

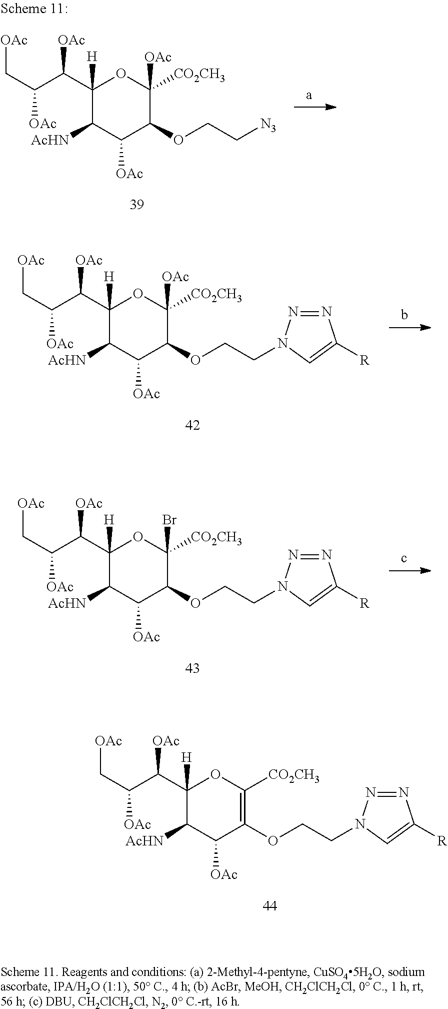

An exemplary method for producing a divalent array of an inhibitor of the invention, where X₅ is a glycerol side-chain [such as described in MacDonald, S. J. F. et al., Antimicrob. Agents Chemother. (2004) 48, 4542-4549], is shown in Scheme 12. Manipulation of the glycerol side-chain of compound (45) to protect the C-8 and C-9 hydroxyl groups as a cyclic carbonate (46) [as described for example in: Andrews, D. M. et al., Eur. J. Med. Chem. (1999) 34, 563-574; MacDonald et al. (2004); Lu and Gervay-Hague, Carbohydr. Res. (2007) 342, 1636-1650.], followed by selective acylation of the C-4 hydroxyl group giving (47), exposes the C-7 hydroxyl group to reaction. Functionalisation from the C-7 hydroxyl group as a carbamate either through direct reaction with a di-isocyanate [as described for example in MacDonald et al. (2004)] or via a p-nitrophenyl ester and subsequent reaction with a diamine [as described for example in MacDonald, S. J. F. et al., J. Med. Chem. (2005) 48, 2964-2971] produces the protected divalent compound (48). Removal of the protecting groups provides the divalent compound (49).

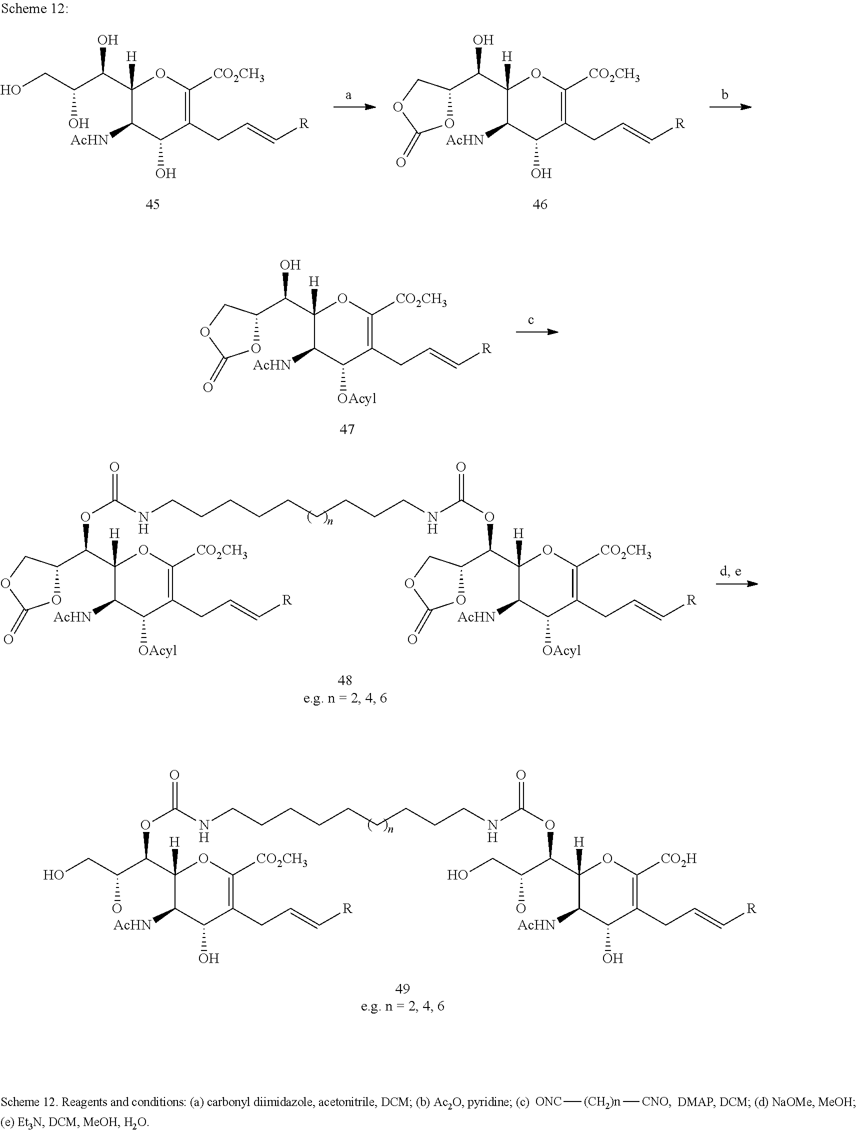

As described above there are a number of general methods for the preparation of the compounds of the invention. In one aspect, general precursors for the preparation of compounds of general formula (I) are compounds of general formula (IV), where Z is a group that, in conjunction with the hydrogen beta to X₆, is removed from (IV) to form an alpha,beta-unsaturated compound (VII), in which X₆ is X₁, or is a functional group that can be subsequently modified to obtain X₁. For example, when X₆ is a functional group that can be modified to form X₁, X₆ can be selected from, but is not limited to, CHO, CH₂OR′, CN, or a thiazole, where R′ is a protecting group. In general, CHO and CH₂OR′ can be converted to X₁, where X₁ is a carboxylate function, using oxidation methods. In general, CN can be converted to X₁, where X₁ is a carboxylate function, by reaction under acidic or basic conditions. In general, a thiazole can be converted to X₁, where X₁ is a carboxylate function, by a series of reactions such as the sequential use of methyl triflate, sodium borohydride, and CuCl₂—CuO (as described for example in Dondoni, A. et al. Tetrahedron (1998) 54, 9859-9874). There are a range of methods for the generation of the alpha,beta-unsaturated compound (VII) from compounds of type (IV), a number of which are described and exemplified in the Methods section.

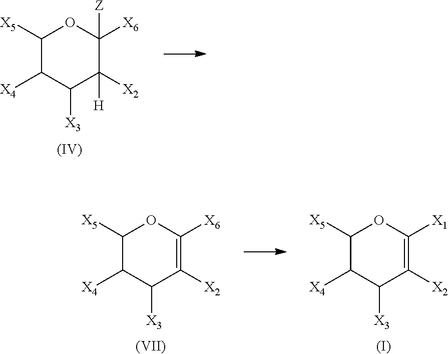

Formation of (VII) from (IV) when Z is halide can be performed, for example, by the use of a base [as described for example in Blattner, R. et al., J. Chem. Soc. Perkin I (1980) 1535-1539; Rye and Withers, J. Org. Chem. (2002) 67, 4505-45121, or by the use of a heavy metal reagent such as a silver or mercury compound [as described for example in Tokuyama and Kenji, Tetrahedron Lett. (1969) 2383-2385; Somsak, L. Carbohydr. Res. (1989) 195, c1-c2]. Formation of (VII) from (IV) when Z is acyloxy can be performed, for example, by the use of a Lewis acid [as described for example in Kok, G. B. et al., Carbohydr. Res. (1996) 289, 67-75]. Formation of (VII) from (IV) when Z is alkoxy can be performed, for example, under acetolysis condition's [as described for example in Kok, G. B. et al., Chem. Commun. (1996) 2017]. Formation of (VII) from (IV) when Z is phosphite can be performed, for example, by the use of a Lewis acid [as described for example in Stolz, F. et al., J. Org. Chem. (2004) 69, 665-679].

Compounds of general formula (IV) where Z is a halide can be formed, as described and exemplified in the Methods section. Compounds of general formula (IV) where Z is a halide can also be formed by halogenation of a compound of the general formula (VI) where Q can be selected from, but is not limited to, —COOR′, —CN, and —CH₂OR′, where R′ is a protecting group, to give (IV) where Z is a halide (as described for example in Blattner, R. et al., J. Chem. Soc. Perkin I (1980) 1535-1539; Rye and Withers, J. Org. Chem. (2002) 67, 4505-4512].

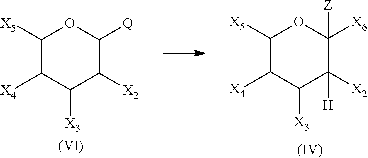

Compounds of general formula I may also be prepared by direct lithiation of a C-2 substituted glycal of general structure (V)(as described for example in Schmidt, R. R. et al. Tetrahedron Lett. (1987) 28, 6591-6594).

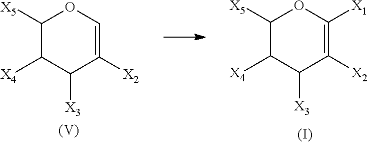

### EXAMPLES

The following examples refer to the schemes above. All new compounds gave the expected spectroscopic data.

### General Method for Base-Catalysed Ester Hydrolysis:

A solution of compound (0.05 mmol) in aqueous MeOH (50%, 4 mL) at 5° C. or room temperature is adjusted to pH 13 using aq. NaOH (1 M). The solution is stirred at a temperature of 5° C. or room temperature and the progress of reaction is monitored by TLC analysis (EtOAc/MeOH/H₂O, 7:2:1). After 2-24 h Amberlite® IR-120 (H⁺) resin is added to adjust pH 3, the reaction mixture is filtered, the resin is washed with MeOH/H₂O 1:1 (25 mL) and the filtrate is concentrated to dryness under vacuum. The crude product is dissolved in water, the pH of the solution is adjusted to pH 7 using aq. NaOH (1 M), and the solution is lyophilised. The product can be purified by reverse phase HPLC.

### General Method for Cross Metathesis Reactions:

To a solution of the allyl derivative (6) (100 mg, 0.19 mmol) in anhydrous dichloromethane (19.5 mL) under N₂, was added olefin (acyclic alkene) (1.94 mmol) followed by Grubbs second generation catalyst (1-15 mol %), and the reaction mixture was stirred at 20-60° C. for 12-60 h. The solvent was removed under vacuum and the crude product was purified by column chromatography on silica (EtOAc-Hexane) to yield the substituted olefin as a white foam.

### Example 1

### Methyl 5-acetamido-4,7,8,9-tetra-O-acetyl-3,5-dideoxy-3-C-(prop-2′-enyl)-D-erythro-β-L-gluco-non-2-ulopyranosonate (3)

To a solution of bromohydrin (2) (1.55 g, 2.71 mmol) [prepared from (1) according to the method of Okamoto et al., 1987] in dry toluene (25 mL) was added allyltributyltin (4.33 g, 13.11 mmol) and azo-bis-isobutyronitrile (AIBN) (44 mg, 0.271 mmol) at room temperature under N₂. The reaction mixture was stirred at room temperature under vacuum for 20 mins, followed by reaction mixture at 100° C. for 8 h (complete disappearance of starting material by TLC analysis). The reaction mixture was concentrated under vacuum, the residue was dissolved in acetonitrile (30 mL), and the solution was washed with petroleum ether (3×20 mL). The acetonitrile extract was concentrated under reduced pressure and the crude product was purified by flash chromatography on silica gel to afford the allyl derivative (3) (Paulsen and Matschulat, 1991) as a white solid (825 mg, 57%).

R_{f} 0.5 (EtOAc); ¹H NMR (300 MHz, CDCl₂): δ 1.85 (NHCOCH₃), 1.98, 1.99, 2.06, 2.10 (4s, 14H, OCOCH₃×4, —CH₃—), 2.48-2.56 (m, 1H, H-3), 3.80 (s, 3H, COOCH₃), 3.97 (dd, J=12.3, 6.9 Hz, 1H, H-9), 4.07 (dd, J=7.2 Hz, 1H, H-6), 4.20 (ddd, J=10.2, 9.9 Hz, 1H, H-5), 4.32 (dd, J=12.6, 2.4 Hz, 1H, H-9′), 4.37 (s, 1H, 2-OH), 4.87-4.93 (m, 2H, ═CH₂), 4.99 (dd, J=9.9 Hz, 1H, H-4), 5.18 (ddd, J=7.8, 5.4, 2.4 Hz, 1H, H-8), 5.28 (dd, J=6.6, 2.1 Hz, 1H, H-7), 5.51-5.65 (m, 2H, NH, —CH═). LRMS (+ ve mode): m/z 554.2 [M+Na]⁺.

### Example 2

### Methyl 5-acetamido-2,4,7,8,9-penta-O-acetyl-3,5-dideoxy-3-C-(prop-2′-enyl)-D-erythro-α-L-gluco-non-2-ulopyranosonate (4)

To a solution of allyl derivative (3) (700 mg, 1.31 mmol) in anhydrous pyridine (16 mL) was added acetic anhydride (8 mL) and 4-(dimethylamino)pyridine (1.5 mg, 1 mol %) at room temperature under N₂. The reaction mixture was stirred at room temperature for 16 h (complete disappearance of starting material by TLC analysis). The reaction mixture was evaporated to dryness, taken up in ethyl acetate (50 mL) and washed successively with 0.1 N HCl, H₂O, and satd aq. NaCl. The organic phase was dried 10, (anhydrous Na₂SO₄), filtered and evaporated under reduced pressure, and the residue purified by flash chromatography on silica gel (EtOAc/hexanes 4:1) to afford the title compound as a white solid (720 mg, 95%).

R_{f} 0.4 (EtOAc); ¹H NMR (300 MHz, CDCl₃): δ 1.78, 1.93, 1.95, 1.97, 2.07, 2.11 (6×s, 18H, NHCOCH₃, OCOCH₃×5), 2.01-2.05 (m, 1H, —CH₂—), 2.12 (m, 1H, H-3), 2.30-2.39 (m, 1H, —CH₂—), 3.73 (s, 3H, COOCH₃), 3.88 (dd, J=10.5, 2.1 Hz, 1H, H-6), 4.05 (dd, J=12.3, 7.2 Hz, 1H, H-9), 4.12 (app. q, J=10.5 Hz, 1H, H-5), 4.52 (dd, J=12.3, 2.4 Hz, 1H, H-9′), 4.81-4.89 (m, 3H, ═CH₂—, H-8), 5.06 (dd, J=10.5, 10.5 Hz, 1H, H-4), 5.30 (dd, J=6.0, 2.7 Hz, 1H, H-7), 5.59 (m, 1H, —CH═), 5.72 (d, J=9.9 Hz, 1H, NH); ¹³C NMR (75.5 MHz, CDCl₃): δ 20.7, 20.9 (OCOCH₃×4), 22.9 (NHCOCH₃), 30.9 (—CH₂—), 45.6 (C-5), 49.2 (C-6), 53.1 (COOCH₃), 62.0 (C-9), 68.0 (C-8), 72.2 (C-4), 72.4 (C-3, C-7), 99.3 (C-2), 115.7 (═CH₂), 135.3 (—CH═), 165.6 (C-1), 167.8 (NHCOCH₃), 170.2, 170.6, 170.9, 171.1 (OCOCH₃×5); LRMS [C₂₅H₃₅NO₁₄] (+ ve ion mode) (m/z): 595.9 [M+Na]⁺, 533.8.

### Example 3

### Methyl 5-acetamido-4,7,8,9-tetra-O-acetyl-2,6-anhydro-3,5-dideoxy-3-C-(prop-2′-enyl)-D-glycero-D-galacto-non-2-enonate (6)

Anhydrous MeOH (3.6 mL, 0.08 mol) was slowly added dropwise to AcCl (10 mL, 0.14 mol) with cooling in an ice-water bath. (Caution! This reaction is exothermic and rapid addition of methanol can result in violent release of HCl gas). The resulting solution was added to a cold solution of glycosyl acetate (4) (225 mg, 0.39 mmol) in a mixture of anhydrous CH₂Cl₂ (10 mL) and AcCl (10 mL, 0.14 mol). The reaction mixture was then stirred at room temperature in a sealed (glass-stoppered) round bottom flask for 48 h. The reaction mixture was concentrated under reduced pressure and azeotroped with toluene (3×20 mL) to yield glycosyl chloride (5) as an off-white foam. The crude chloride was taken up in dry dichloromethane (10 mL), to which DBU (232 microL, 1.56 mmol, 4 mole equiv.) was added, and the reaction was left to stir at room temperature under N₂ for 8 h. The reaction mixture was evaporated to dryness, taken up in chloroform and washed successively with satd aq. NH₄Cl, H₂O, and satd aq. NaCl. The organic phase was dried (anhydrous Na₂SO₄), filtered and evaporated under reduced pressure, and the residue purified by flash chromatography on silica gel (EtOAc/hexanes 3:2) to afford the title compound as a white solid (93 mg, isolated yield 46%, corrected yield over 2 steps 91% based on recovered starting material).

R_{f} 0.6 (EtOAc); ¹H NMR (300 MHz, CDCl₃): δ 1.85, 2.00, 2.02, 2.03, 2.07 (5×s, 15H, NHCOCH₃, OCOCH₃×4), 2.91 (dd, J=15.0, 6.9 Hz, 1H, —CH₂—), 3.32 (dd, J=15.0, 6.0 Hz, 1H, —CH₂—), 3.74 (s, 3H, COOCH₃), 4.10 (dd, J=12.3, 7.2 Hz, 1H, H-9), 4.18 (dd, J=9.6, 3.3 Hz, 1H, H-6), 4.38 (ddd, J=9.6, 8.1, 8.4 Hz, 1H, H-5), 4.59 (dd, J=12.3, 2.7 Hz, 1H, H-9′), 4.97 (dd, J=13.5, 2.1 Hz, 2H, ═CH₂), 5.22 (m, 1H, H-8), 5.44 (dd, J=5.1, 3.3 Hz, 1H, H-7), 5.50 (d, J=9.9 Hz, 1H, NH), 5.55 (d, J=7.8 Hz, 1H, H-4), 5.62-5.76 (m, 1H, —CH═); ¹³C NMR (75.5 MHz, CDCl₃): δ 20.7, 20.8 (OCOCH₃×4), 23.0 (NHCOCH₃), 31.5 (—CH₂—), 47.5 (C-5), 52.2 (COOCH₃), 62.0 (C-9), 68.2 (C-7), 70.4 (C-4), 71.0 (C-8), 76.2 (C-6), 116.3 (═CH₂), 120.2 (C-3), 134.9 (—CH═), 141.4 (C-2), 162.2 (C-1), 169.9, 170.1, 170.2, 170.5, 171.1 (NHCOCH₃, OCOCH₃×4). LRMS [C₂₃H₃₁NO₁₂] (+ ve ion mode) m/z: 536.2 [M+Na]⁺, 476.2, 416.1, 231.9.

### Example 4

### 5-Acetamido-2,6-anhydro-3,5-dideoxy-3-C-(prop-2′-enyl)-D-glycero-D-galacto-non-2-en-onic acid (7)

Compound (6) was deprotected according to the general procedure at 5° C. for 12 h. The crude product was purified by reverse phase HPLC and then lyophilized to give the title compound as a white solid (32 mg, isolated yield 51%, corrected yield 60% based on recovered starting material).

R_{f} 0.1 (EtOAc/MeOH/H₂O, 7:2:1); ¹H NMR (300 MHz, D₂O): δ 2.03 (s, 3H, NHCOCH₃), 3.07 (dd, J=15.3, 6.9 Hz, 1H, —CH₂—), 3.31 (dd, J=15.3, 5.1 Hz, 1H, —CH₂—), 3.58-3.66 (m, 2H, H-7, H-9), 3.82-3.89 (m, 2H, H-8, H-9′), 4.09-4.18 (m, 2H, H-5, H-6), 4.31 (dd, J=6.6, 2.4 Hz, 1H, H-4), 5.03-5.14 (m, 2H, ═CH₂), 5.79-5.93 (m, 1H, —CH═); ¹³C NMR (75.5 MHz, D₂O): δ 22.0 (NHCOCH₃), 30.2 (—CH₂—), 50.5 (C-5), 62.9 (C-9), 68.1 (C-7), 68.9 (C-4), 69.8 (C-8), 75.4 (C-6), 115.7 (═CHO, 119.9 (C-3) 135.9 (—CH═), 174.6 (NHCOCH₃) (C-1 and C-2 not observed); LRMS [C₂₄H₂₂NO₈] m/z (+ ve ion mode): 354 [M+Na]⁺; (−ve mode) 330 [M-1]⁺; HRMS (FAB): Calc. for C₂₄H₂₂NO₈: 330.119441. Found: m/z 330.118000.

### Example 5

### Methyl 5-acetamido-4,7,8,9-tetra-O-acetyl-2,6-anhydro-3,5-dideoxy-3-C-(4,4-dimethylpent-2′-enyl)-D-glycero-D-galacto-non-2-enonate (8a, R=t-butyl)

To a solution of the allyl derivative (6) (120 mg, 0.23 mmol) in anhydrous dichloromethane (20 mL) under N₂, was added 3,3-dimethyl-1-butene (0.29 mL, 2.33 mmol) followed by Grubbs second generation catalyst (28 mg, 15 mol %), and the reaction mixture was stirred at 40° C. for 24 h. The solvent was removed under vacuum and the crude product was purified by column chromatography on silica (EtOAc-Hexane) to yield the title compound (8a) as a white foam (52 mg, 39%; corrected yield 59% based on recovered starting material).

R_{f} 0.7 (EtOAc); ¹H NMR (300 MHz, CDCl₃): δ 0.94 (s, 9H, C(CH₃)₃, 1.87 (s, 3H, NHCOCH₃), 2.02, 2.04, 2.05, 2.09 (4×s, 12H, OCOCH₃), 2.78 (dd, J=14.7, 7.8 Hz, 1H, —CH₂—), 3.39 (dd, J=14.7, 6.9 Hz, 1H, —CH₂—), 3.76 (s, 3H, COOCH₃), 4.12 (dd, J=12.3, 7.2 Hz, 1H, H-9), 4.17 (dd, J=9.6, 3.3 Hz, 1H, H-6), 4.41 (ddd, J=9.6, 8.1, 7.8 Hz, 1H, H-5), 4.62 (dd, J=12.3, 3.0 Hz, 1H, H-9′), 5.17 (m, 1H, ═CH—), 5.25 (m, 1H, H-8), 5.33 (d, J=9.9 Hz, 1H, NH), 5.44-5.47 (m, 2H, H-7, —CH═), 5.55 (d, J=9.9 Hz, 1H, H-4); LRMS [C₂₇H₃₉NO₁₂] (+ ve ion mode) m/z: 592.2 [M+Na]⁺.

### Example 6

### 5-Acetamido-2,6-anhydro-3,5-dideoxy-3-C-(4,4-dimethylpent-2′-enyl)-D-glycero-D-galacto-non-2-enonic acid (9a, R=t-butyl)

Compound (8a, R=t-butyl) was deprotected according to the general procedure at room temperature for 24 h. The crude product was purified by reverse phase HPLC and then lyophilized to give title compound (9a) as a white solid (9 mg, 53%).

R_{f} 0.1 (EtOAc/MeOH/H₂O, 7:2.5:0.5); ¹H NMR (300 MHz, O₂O): δ 0.82 (s, 9H, C(CH₃)₃, 1.89 (s, 3H, NHCOCH₃), 2.83 (dd, J=14.7, 7.5 Hz, 1H, —CH₂—), 3.17 (dd, J=15.0, 5.7 Hz, 1H, —CH₂—), 3.45-3.51 (m, 2H, H-7, H-9), 3.68-3.74 (m, 2H, H-8, H-9′), 3.99-4.01 (m, 2H, C-5, H-6), 4.16 (dd, J=6.3, 3.0 Hz, 1H, H-4), 5.17-5.27 (m, 1H, —CH═), 5.49 (d, J=15.6 Hz, 1H, ═CH—); ¹³C NMR (75.5 MHz, D₂O): δ 24.4 (NHCOCH₃), 31.24 (C(CH₃)₃), 31.6 (—CH₂—), 34.6 (C(CH₃)₃), 53.0 (C-5), 65.3 (C-9), 70.5 (C-7), 71.1 (C-4), 72.3 (C-8), 77.9 (C-6), 123.7 (═CH—), 125.0 (C-3), 142.9 (C-2), 146.7 (—CH═), 170.0 (C-1), 177.0 (NHCOCH₃). LRMS [C₁₈H₂₉NO₈] m/z (− ve ion mode): 386.1 [M-1]⁺; HRMS (FAB): Calc. for C₁₈H₂₉N₁O₈Na₁ (+1): 410.178538. Found: m/z 410.179200.

### Example 7

### Methyl 5-acetamido-4,7,8,9-tetra-O-acetyl-2,6-anhydro-3,5-dideoxy-3-C-(3′-cyclohexyl-prop-2′-enyl)-D-glycero-D-galacto-non-2-enonate (8b, R=cyclohexyl)

To a solution of the allyl derivative (6) (100 mg, 0.19 mmol) in anhydrous dichloromethane (19.5 mL) under N₂, was added vinyl cyclohexane (0.26 mL, 1.94 mmol) followed by Grubbs second generation catalyst (19 mg, 0.023 mmol, 12 mol %), and the reaction mixture was stirred at 40° C. for 48 h. The solvent was removed under vacuum and the crude product was purified by column chromatography on silica (EtOAc-Hexane) to yield the title compound (8b) as a white foam (52 mg, isolated yield 45%, corrected yield 64% based on recovered starting material).

R_{f} 0.65 (EtOAc); ¹H NMR (300 MHz, CDCl₃): δ 1.00-1.27 (m, 5H, cyclohexyl), 1.65 (m, 5H, cyclohexyl), 1.86, 2.02, 2.04, 2.08 (5s, 16H, NHCOCH₃, OCOCH₃×4, cyclohexyl-CH), 2.77 (dd, J=15.0, 7.8 Hz, 1H, —CH₂—), 3.35 (dd, J=14.7, 5.1 Hz, 1H, —CH₂—), 3.75 (s, 3H, COOCH₃), 4.12 (dd, J=12.3, 6.9 Hz, 1H, H-9), 4.17 (dd, J=9.6, 3.3 Hz, 1H, H-6), 4.41 (ddd, J=15.9, 6.3 Hz, 1H, H-5), 4.62 (dd, J=12.3, 3.0 Hz, 1H, H-9′), 5.22-5.28 (m, 2H, H-8, —CH═), 5.30 (d, J=6.6 Hz, 1H, H-7), 5.40-5.46 (m, 2H, H-4, ═CH—), 5.53 (d, J=8.1 Hz, 1H, NH); LRMS [C₂₉H₄₁NO₁₂] m/z (+ ve ion mode): 618.1 [M+Na]⁺.

### Example 8

### 5-Acetamido-2,6-anhydro-3,5-dideoxy-3-C-(3′-cyclohexyl-prop-2′-enyl)-D-glycero-D-galacto-non-2-enonic acid (9b, R=cyclohexyl)

Compound (8b, R=cyclohexyl) was deprotected according to the general procedure at room temperature for 24 h. The crude product was purified by reverse phase HPLC and then lyophilized to give title compound (9b) as white solid (12 mg, isolated yield 33%).

R_{f} 0.2 (EtOAc/MeOH/H₂O, 7:2:1); ¹H NMR (300 MHz, D₂O): δ 0.82-1.15 (m, 5H, cyclohexyl), 1.41-1.52 (m, 5H, cyclohexyl), 1.76-1.78 (m, 1H, cyclohexyl-CH), 1.88 (s, 3H, NHCOCH₃), 2.72 (dd, J=14.7, 7.5 Hz, 1H, —CH₂—), 2.97 (dd, J=14.7, 5.4 Hz, 1H, —CH₂—), 3.41-3.49 (m, 2H, H-7, H-9), 3.67-3.73 (m, 2H, H-8, H-9′), 3.95-3.97 (m, 2H, H-5, H-6), 4.10 (dd, J=6.6, 2.4 Hz, 1H, H-4), 5.19-5.28 (m, 1H, —CH═), 5.40 (dd, J=15.6, 6.3 Hz, 2H, ═CH—); ¹³C NMR (75.5 MHz, D₂O): δ 21.9 (NHCOCH₃), 25.5, 25.7 (C cyclohexyl), 29.1 (—CH₂—), 32.4 (C cyclohexyl), 39.9 (—CH-cyclohexyl), 50.7 (C-5), 62.87 (C-9), 68.2 (C-7), 68.4 (C-4), 69.6 (C-8), 75.2 (C-6), 115.9 (C-3), 124.2 (═CH—), 139.0 (—CH═), 169.0 (C-1), 174.4 (NHCOCH₃) (C-2 not observed). LRMS [C₂₀H₃₁NO₈] m/z (+ ve ion mode): 436.2 [M+Na]⁺, 396.2, 319.2, 218.6, 179.4, 133.8; m/z (− ve ion mode): 412.2 [M-H]⁺, 340.0, 269.0, 199.9, 164.1; HRMS (FAB): Calc. for C₂₀H₃₁N₁O₈Na₁ (+1): 436.195178. Found: m/z 436.194188.

### Example 9

### Methyl 5-acetamido-4,7,8,9-tetra-O-acetyl-2,6-anhydro-3,5-dideoxy-3-C-(3′-phenyl-prop-2′-enyl)-D-glycero-D-galacto-non-2-enonate (8c, R=Ph)

To a solution of the allyl derivative (6) (100 mg, 0.19 mmol) in anhydrous dichloromethane (19.5 mL) under N₂, was added styrene (0.22 mL, 1.94 mmol) followed by Grubbs second generation catalyst (19 mg, 0.023 mmol, 12 mol %) and the reaction was stirred at 40° C. for 22 h. The solvent was removed under vacuum and the crude product was purified by column chromatography on silica (EtOAc-Hexane) to yield the title compound (8c) as a white foam (30 mg, isolated yield 26%, corrected yield 64% based on recovered starting material).

R_{f} 0.7 (EtOAc); ¹H NMR (300 MHz, CDCl₃): δ 1.86, 2.02, 2.03, 2.04, 2.10 (5s, 15H, NHCOCH₃, OCOCH₃×4), 3.12 (dd, J=15.0, 7.2 Hz, 1H, —CH₂—), 3.44 (dd, J=15.3, 6.9 Hz, 1H, —CH₂—), 3.78 (s, 3H, COOCH₃), 4.08-4.22 (m, 2H, H-6, H-9′), 4.43 (ddd, J=9.3, 7.8, 1.5 Hz, 1H, H-5), 4.61 (dd, J=12.3, 2.7 Hz, 1H, H-9), 5.24-5.29 (m, 1H, H-8), 5.45-5.48 (m, 2H, H-4, H-7), 5.61 (d, J=7.5 Hz, 1H, NH), 6.04-6.14 (m, 1H, ═CH—), 6.34 (d, J=15.9 Hz, 1H, Ph-CH═), 7.15-7.31 (m, 5H, Ph); ¹³C NMR (75.5 MHz, CDCl₃): δ 20.7, 20.8, 20.9 (OCOCH₃×4), 23.1 (NHCOCH₃), 31.0 (—CH₂—), 47.6 (C-5), 52.3 (COOCH₃), 62.0 (C-9), 67.3 (C-7), 70.4 (C-4), 70.8 (C-8), 76.2 (C-6), 120.3 (C-3), 126.1 (═CH—), 126.5, 127.2, 128.5 (ArC), 131.7 (ArCH═), 137.1 (C-2), 141.4 (Ar q carbon), 162.3 (C-1), 170.0, 170.1, 170.6, 171.1 (NHCOCH₃, OCOCH₃); LRMS [C₂₉H₃₅NO₁₂] m/z (+ ve ion mode): 612.2 [M+Na]⁺.

### Example 10

### 5-Acetamido-2,6-anhydro-3,5-dideoxy-3-C-(3′-phenyl-prop-2′-enyl)-D-glycero-D-galacto-non-2-enonic acid (9c, R=Ph)

Compound (8c, R=Ph) was deprotected according to the general procedure at room temperature for 24 h. The crude product was purified by reverse phase HPLC and then lyophilized to give title compound (9c) as a white solid (8 mg, isolated 40%).

R_{f} 0.2 (EtOAc/MeOH/H₂O, 7:2:1); ¹H NMR (300 MHz, D₂O): δ 1.88 (s, 3H, NHCOCH₃), 3.1 (dd, J=15.3, 7.5 Hz, 1H, —CH₂—), 3.3 (dd, J=14.7, 5.4 Hz, 1H, —CH₂—), 3.45-3.51 (m, 2H, H-7, H-9), 3.68-3.75 (m, 2H, H-8, H-9′), 4.01-4.03 (m, 2H, H-5, H-6), 4.21 (dd, J=6.0, 3.0 Hz, 1H, H-4), 6.12-6.22 (m, 1H, ═CH—), 6.38 (d, J=15.9 Hz, 1H, Ar—CH═), 7.09-7.21 (m, 5H, ArH); ¹³C NMR (75.5 MHz, D₂O): δ 21.9 (NHCOCH₃), 29.5 31.0 (—CH₂—), 50.5 (C-5), 62.9 (C-9), 68.0 (C-7), 69.0 (C-4), 69.8 (C-8), 75.4 (C-6), 120.6 (C-3), 125.9 (═CH—), 127.3, 128.0, 128.8 (ArC), 130.7 (Ar—CH═), 137.3 (C-2), 174.6 (NHCOCH₃) (C-1 not observed). LRMS [C₂₀H₂₅NO₈] m/z (+ ve ion mode): 430.1 [M+Na]⁺, 368.1, 276.9, 237.8; m/z (− ve ion mode): 406.1 [M-1]⁺, 362.1, 308.1, 284.1, 235.9, 168.9, 140.9.; HRMS (FAB): Calc. for C₂₀H₂₅N₃O₈Na₁ (+1): 430.147238. Found: m/z 430.148173.

### Example 11

### Methyl 5-acetamido-4,7,8,9-tetra-O-acetyl-2,6-anhydro-3,5-dideoxy-3-C-[3′-(p-tolyl)-prop-2′-enyl]-D-glycero-D-galacto-non-2-enonate (8d, R=4-CH₃Ph)

To a solution of the allyl derivative (6) (93 mg, 0.18 mmol) in anhydrous dichloromethane (18 mL) under N₂, was added 4-methylstyrene (0.23 mL, 1.80 mmol) followed by Grubbs second generation catalyst (22 mg, 0.027 mmol, 15 mol %) and the reaction was stirred at 40° C. for 22 h. The solvent was removed under vacuum and the crude product was purified by column chromatography on silica (EtOAc-Hexane) to yield the title compound (8d) as a white foam (75 mg, isolated yield 69%, corrected yield 77% based on recovered starting material).

R_{f} 0.7 (EtOAc); ¹H NMR (300 MHz, CDCl₃): δ 1.85, 2.02, 2.03, 2.09 (4×s, 15H, NHCOCH₃, OCOCH₃×4), 2.28 (s, 3H, p-tolyl CH₃), 3.09 (dd, J=14.7, 7.2 Hz, 1H, —CH2—), 3.42 (dd, J=14.4, 6.6 Hz, 1H, —CH₂—), 3.78 (s, 3H, COOCH₃), 4.12 (dd, J=12.3, 6.9 Hz, 1H, H-9), 4.19 (dd, J=9.6, 3.3 Hz, 1H, H-6), 4.43 (ddd, J=9.6, 9.3, 8.1 Hz, 1H, H-5), 4.61 (dd, J=12.3, 2.7 Hz, 1H, H-9′), 5.25 (m, 1H, H-8), 5.46 (m, 1H, H-7), 5.54 (d, J=9.6 Hz, 1H, NH), 5.60 (d, J=7.8 Hz, 1H, H-4), 6.04 (m, 1H, 6.30 (d, J=15.9 Hz, 1H, ═CHAr), 7.06 (d, J=8.1 Hz, 2H, Ar), 7.18 (d, J=8.1 Hz, 2H, Ar); ¹³C NMR (75.5 MHz, CDCl₃): δ 20.7, 20.8, 20.9, 21.1 (OCOCH₃×4), 23.1 (NHCOCH₃, p-tolyl CH₃), 31.0 (—CH₂—), 47.5 (C-5), 52.2 (COOCH₃), 62.0 (C-9), 67.3 (C-7), 70.5 (C-4), 70.8 (C-8), 76.2 (C-6), 120.5 (C-3), 125.4 (—CH═), 126.0 (Ar), 129.2 (Ar), 131.5 (═CH—Ar), 134.4 (Ar q carbon), 137.0 (Ar q carbon), 141.4 (C-2), 162.3 (C-1), 170.0, 170.1, 170.5, 171.1 (NHCOCH₃, OCOCH₃×4); LRMS [C₃₀H₃₇NO₁₂] m/z (+ ve ion mode): 626.2 [M+Na]⁺, 588.2, 536.0, 440.0, 262.0

### Example 12

### 5-Acetamido-2,6-anhydro-3,5-dideoxy-3-C-13′-(p-tolyl)-prop-2′-enyl)-D-glycero-D-galacto-non-2-enonic acid (9d, R=4-CH₃Ph)

Compound (8d, R=4-CH₃Ph) was deprotected according to the general procedure at room temperature for 24 h. The crude product was purified by reverse phase HPLC and then lyophilized to give title compound (9d) as a white solid (45 mg, isolated 94%).

R_{f} 0.3 (EtOAc/MeOH/H₂O, 7:2:1); ¹H NMR (300 MHz, D₂O): δ 1.98 (s, 3H, NHCOCH₃), 2.25 (p-tolyl CH₃), 3.15 (dd, J=15.3, 7.5 Hz, 1H, —CH₂—), 3.40 (dd, J=14.7, 6.0 Hz, 1H, —CH₂—), 3.54-3.61 (m, 2H, H-7, H-9), 3.79-3.86 (m, 2H, H-8, H-9′), 4.11-4.13 (m, 2H, H-5, H-6), 4.30 (dd, J=4.5, 4.2 Hz, 1H, H-4), 6.17-6.26 (m, 1H, —CH═), 6.45 (d, J=15.9 Hz, 1H, ═CH—Ar), 7.16 (d, J=8.1 Hz, 2H, Ar), 7.30 (d, J=8.4 Hz, 2H, Ar); ¹³C NMR (75.5 MHz, D₂O): δ 20.0 (p-tolyl CH₃), 21.9 (NHCOCH3), 29.4 (—CH₂—), 50.4 (C-5), 62.8 (C-9), 67.9 (C-7), 68.9 (C-4), 69.7 (C-8), 75.3 (C-6), 121.4 (C-3), 125.9 (Ar), 126.8 (—CH₂—CH═), 129.3 (Ar), 130.5 (═CHAr), 134.4 (Ar q carbon), 137.6 (Ar q carbon), 141.0 (C-2), 174.5 (NHCOCH₃) (C-1 not observed). LRMS [C₂₁H₂₇NO₈] m/z (− ve ion mode): 420.1 [M-1]⁺; HRMS (FAB): Calc. for C₂₁H₂₇NlO₈Na₁: 444.162888. Found: m/z 444.164115.

### Example 13

### Methyl 5-acetamido-4,7,8,9-tetra-O-acetyl-2,6-anhydro-3,5-dideoxy-3-C-[3′-(4-tert-butoxyphenyl)-prop-2′-enyl]-D-glycero-D-galacto-non-2-enonate [8e, R=4-(t-butoxy)Ph]

To a solution of the allyl derivative (6) (75 mg, 0.14 mmol) in anhydrous dichloromethane (18 mL) under N₂, was added 4-(tert-butoxy)styrene (0.27 mL, 1.46 mmol) followed by Grubbs second generation catalyst (17.8 mg, 0.021 mmol, 15 mol %) and the reaction was stirred at 40° C. for 24 h. The solvent was removed under vacuum and the crude product was purified by column chromatography on silica (EtOAc-Hexane) to yield the title compound (8e) as a white foam (30 mg, isolated yield 31%, corrected yield 59% based on recovered starting material).

R_{f} 0.7 (EtOAc); ¹H NMR (300 MHz, CDCl₃): δ 1.30 (s, 9H, C(CH₃)₃), 1.87, 2.02, 2.04, 2.10 (5×s, 15H, NHCOCH₃, OCOCH₃×4), 3.06 (dd, J=14.4, 7.2 Hz, 1H, —CH₂—), 3.46 (dd, J=14.4, 6.9 Hz, 1H, —CH₂—), 3.78 (s, 3H, COOCH₃), 4.12 (dd, J=12.6, 6.9 Hz, 1H, H-9), 4.19 (dd, J=9.3, 3.3 Hz, 1H, H-6), 4.43 (ddd, J=9.6, 7.8, 7.8 Hz, 1H, H-5), 4.60 (dd, J=12.6, 3.0 Hz, 1H, H-9′), 5.26 (m, 1H, H-8), 5.37 (d, J=9.3 Hz, 1H, 5.46 (dd, J=5.1, 3.3 Hz, 1H, H-7), 5.61 (d, J=7.5 Hz, 1H, H-4), 5.99 (m, 1H, —CH═), 6.30 (d, J=15.9 Hz, 1H, ═CH—Ar), 6.88 (d, J=8.4 Hz, 2H, ArH), 7.20 (d, J=8.4 Hz, 2H, ArH); LRMS [C₃₃H₄₃NO₂₃] m/z (+ ve ion mode): 684.2 [M+Na]⁺.

### Example 14

### 5-Acetamido-2,6-anhydro-3,5-dideoxy-3-C-[3′-(4-tert-butoxyphenyl)-prop-2′-enyl]-D-glycero-D-galacto-non-2-enonanic acid [9e, R=4-(t-butoxy)Ph]

Compound [8e, R=4-(t-butoxy)Ph] was deprotected according to the general procedure at room temperature for 24 h. The crude product was purified by reverse phase HPLC and then lyophilized to give title compound (9e) as a white solid (11 mg, isolated 61%).

R_{f} 0.3 (EtOAc/MeOH/H₂O, 6:3.5:0.5); ¹H NMR (300 MHz, D₂O): δ 1.11 (s, 9H, C(CH₃)₃, 2.81 (s, 3H, NHCOCH₃), 2.91 (dd, J=15.6, 8.4 Hz, 1H, —CH₂—), 3.11 (dd, J=15.0, 5.7 Hz, 1H, —CH₂—), 3.35-3.44 (m, 2H, H-7, H-9), 3.62-3.69 (m, 2H, H-8, H-9′), 3.92-3.95 (m, 2H, H-5, H-6), 4.08 (dd, J=6.6, 2.4 Hz, 1H, H-4), 6.02 (m, 1H, —CH═), 6.28 (d, J=15.9 Hz, 1H, ═CHAr), 6.83 (d, J=8.4 Hz, 2H, ArH), 7.18 (d, J=8.7 Hz, 2H, ArH); LRMS [C₂₄H₃₃NO₉] m/z (− ve ion mode): 477.8 [M-1]⁺; HRMS (FAB): Calc. for C₂₄H₃₃N₁O₉Na1 (+1): 502.204753. Found: m/z 502.207250.

### Example 15

### Methyl 5-acetamido-4,7,8,9-tetra-O-acetyl-2,6-anhydro-3,5-dideoxy-3-C-(3′-naphthyl-prop-2′-enyl)-D-glycero-D-galacto-non-2-enonate [8f, R=naphthyl]

To a solution of the allyl derivative (6) (100 mg, 0.19 mmol) in anhydrous dichloromethane (19.5 mL) under N₂, was added 2-vinyl naphthalene (0.29 mg, 1.94 mmol) followed by Grubbs second generation catalyst (24.6 mg, 0.029 mmol, 15 mol %) and the reaction was stirred at 40° C. for 26 h. The solvent was removed under vacuum and the crude product was purified by column chromatography on silica (EtOAc-Hexane) to yield the title compound (8f) as a white foam (92 mg, isolated yield 74%).

R_{f} 0.6 (Toluene:EtOAc, 1:4); ¹H NMR (300 MHz, CDCl₃): δ 1.87 (s, 3H, NHCOCH₃), 2.02, 2.03, 2.04, 2.11 (4×s, 12H, OCOCH₃), 3.20 (dd, J=15.0, 6.9 Hz, 1H, —CH₂—), 3.48 (dd, J=15.0, 6.6 Hz, 1H, —CH₂—), 3.80 (s, 3H, COOCH₃), 4.13 (dd, J=12.3, 7.2 Hz, 1H, H-9), 4.22 (dd, J=9.3, 3.6 Hz, 1H, H-6), 4.46 (ddd, J=9.6, 7.8, 7.8 Hz, 1H, H-5), 4.61 (dd, J=12.3, 3.0 Hz, 1H, H-9′), 5.27 (m, 1H, H-8), 5.40 (d, J=9.6 Hz, 1H, NH), 5.48 (dd, J=5.1, 3.6 Hz, 1H, H-7), 5.65 (d, J=7.8 Hz, 1H, H-4), 6.23 (m, 1 H, —CH═), 6.51 (d, J=15.9 Hz, 1H, ═CH—Ar), 7.39-7.43 (m, 2H, ArH), 7.53 (dd, J=8.7, 1.8 Hz, 1H, ArH), 7.64 (s, 1H, ArH), 7.74-7.77 (m, 3H, ArH); ¹³C NMR (75.5 MHz, CDCl₃): δ 20.7, 20.8, 20.9 (OCOCH₃×4), 23.1 (NHCOCH₃), 31.21 (—CH₂—), 47.5 (C-5), 52.3 (COOCH₃), 61.9 (C-9), 67.3 (C-7), 70.5 (C-4), 70.8 (C-8), 76.2 (C-6), 120.3 (C-3), 123.4 (—CH═), 125.6, 125.8, 126.1, 126.9, 127.6, 127.8, 128.1 (ArC), 131.7 (═CH—Ar), 132.7, 133.5, 134.5 (Ar q carbon), 141.4 (C-2), 162.3 (C-1), 170.0, 170.1, 170.5, 171.1 (NHCOCH₃, OCOCH₃×4); LRMS [C₃₃H₃₇NO₁₂] m/z (+ ve ion mode): 662.2 [M+Na]⁺, 630.3, 602.2.

### Example 16

### 5-Acetamido-2,6-anhydro-3,5-dideoxy-3-C-(3′-naphthyl-prop-2′-enyl)-D-glycero-D-galacto-non-2-enonic acid [9f, R=naphthyl]

Compound (8f, R=naphthyl) was deprotected according to the general procedure at room temperature for 16 h. The crude product was purified by reverse phase HPLC and then lyophilized to give title compound (9f) as a white solid (47 mg, isolated 83%).

R_{f} 0.2 (EtOAc/MeOH/H₂O, 7:2.5:0.5); ¹H NMR (300 MHz, CD₃OD): δ 2.01 (NHCOCH₃), 3.33 (dd, J=8.4 Hz, 1H, —CH₂—), 3.56 (d, J=9.0 Hz, 1H, H-7), 3.65 (dd, J=11.4, 5.4 Hz, 1H, H-9), 3.75-3.88 (m, 3H, H-8, H-9′, 1H, —CH₂—), 4.06-4.16 (m, 2H, H-5, H-6), 4.37 (d, J=7.5 Hz, 1H, H-4), 6.41 (m, 1H, 6.68 (d, J=15.9 Hz, 1H, ═CH—), 7.37-7.45 (m, 2H, ArH), 7.60 (dd, J=8.7, 1.5 Hz, 1H, ArH), 7.69 (s, 1H, ArH), 7.74-7.79 (m, 3H, ArH); ¹³C NMR (75.5 MHz, CD₃OD): δ 21.2 (NHCOCH₃), 29.4 (—CH₂—), 51.4 (C-5), 63.3 (C-9), 68.4 (C-7), 68.7 (C-4), 69.8 (C-8), 76.0 (C-6), 122.3 (C-3), 123.4 (—CH═), 125.2, 125.8, 127.1, 127.4, 127.6, 127.9 (ArC), 131.2 (═CH—Ar), 132.8, 133.7, 135.1 (Ar q carbon), 162.3 (C-1), 173.5 (NHCOCH₃): LRMS [C₂₄H₂₇NO₈] m/z (+ ve ion mode): 480.1 [M+Na]⁺, 440.1; m/z (− ve ion mode) 456.1 [M-H]⁺, 412.1, 334.0, 304.0, 236.9.

### Example 17

### Methyl 5-acetamido-4,7,8,9-tetra-O-acetyl-2,6-anhydro-3,5-dideoxy-3-C-[4′-(3,4-dimethoxyphenyl)-but-2′-enyl]-D-glycero-D-galacto-non-2-enonate [8g, R=3,4-dimethoxybenzyl]

To a solution of the allyl derivative (6) (200 mg, 0.38 mmol) in anhydrous dichloromethane (39 mL) under N₂, was added 4-allyl-1,2-dimethoxybenzene (0.66 mL, 3.89 mmol) followed by Grubbs second generation catalyst (39 mg, 0.046 mmol, 12 mol %) and the reaction was stirred at 40° C. for 48 h. The solvent was removed under vacuum and the crude product was purified by column chromatography on silica (EtOAc-Hexane) to yield the title compound (8g) as a white foam (30 mg, isolated yield 12%, corrected yield 47% based on recovered starting material).

R_{f} 0.35 (EtOAc); ¹H NMR (300 MHz, CDCl₃): δ 1.86, 1.98, 2.02, 2.03 (4×s, 15H, NHCOCH₃, OCOCH₃×4), 2.87 (dd, J=14.7, 7.2 Hz, 1H, —CH₂—), 3.23 (d, J=6.6 Hz, 2H, —CH₂—), 3.34 (dd, J=15.3, 5.7 Hz, 1H, —CH₂—), 3.74 (s, 3H, COOCH3), 3.82, 3.85 (OCH₃×2), 4.12 (dd, J=14.4, 6.9 Hz, 1H, H-9), 4.20 (dd, J=8.7, 5.1 Hz, 1H, H-6), 4.40 (ddd, J=9.6, 7.8, 7.8 Hz, 1H, H-5), 4.61 (dd, J=12.3, 2.7 Hz, 1H, H-9′), 5.25 (m, 1H, H-8), 5.36 (d, J=9.6 Hz, 1H, NH), 5.42-5.52 (m, 3H, H-7, —CH═, ═CH—), 5.56 (d, J=7.8 Hz, 1H, H-4); ¹³C NMR (75.5 MHz, CDCl₃): δ 20.6, 20.7, 20.8 (OCOCH₃×4), 23.0 (NHCOCH₃), 30.3 (—CH₂—), 38.4 (—CH₂—), 47.4 (C-5), 52.1 (COOCH₃), 55.8, 55.9 (OCH₃×2), 62.0 (C-9), 67.4 (C-7), 70.4 (C-4), 71.0 (C-8), 76.2 (C-6), 111.2, 111.8, 120.2 (ArC), 120.6 (C-3), 127.4 (—CH═CH—), 131.4 (—CH═CH—), 133.0 (Ar q carbon), 141.1 (C-2), 147.2, 148.8 (Ar q carbon), 162.3 (C-1), 169.9, 170.0, 170.1, 170.2, 170.5 (OCOCH₃×4, NHCOCH₃); LRMS [C₃₂H₄₁NO₁₄] m/z (+ ve ion mode): 686.2 [M+Na]⁺.

### Example 18

### 5-Acetamido-2,6-anhydro-3,5-dideoxy-3-C-[4′-(3,4-dimethoxyphenyl)-but-2′-enyl]-D-glycero-D-galacto-non-2-enonic acid [9g, R=3,4-dimethoxybenzyl]

Compound (8g, R=3,4-dimethoxybenzyl) was deprotected according to the general procedure at room temperature for 24 h. The crude product was purified by reverse phase HPLC and then lyophilized to give title compound (9g) as a white solid (18 mg, isolated 86%).

R_{f} 0.2 (EtOAc/MeOH/H₂O, 6:3:1); ¹H NMR (300 MHz, D₂O): δ 1.84 (NHCOCH₃), 2.86 (dd, J=15.6, 6.6 Hz, 1H, —CH₂—), 3.10-3.15 (m, 3H, —CH₂—, —CH₂—Ar), 3.46-3.51 (m, 2H, H-7, H-9), 3.65 (s, 6H, 2×OMe), 3.67-3.71 (m, 2H, H-8, H-9′), 3.91-3.97 (m, 2H, H-5, H-6), 4.07-4.21 (m, 1H, H-4), 5.38 (m, 1H, ═CH—), 5.53 (m, 1H, —CH═), 6.65 (d, J=8.1 Hz, 1H, ArH), 6.76 (d, J=2.1 Hz, 1H, 6.81 (d, J=8.1 Hz, 1H, ArH); ¹³C NMR (75.5 MHz, D₂O): δ 21.9 (NHCOCH₃), 28.9 (—CH₂—), 37.5 (—CH₂—), 50.5 (C-5), 55.6, 55.7 (2×OMe), 62.8 (C-9), 68.1 (C-7), 68.8 (C-4), 69.8 (C-8), 75.3 (C-6), 112.0, 112.2, 120.7 (ArC), 128.3 (═CH—), 130.9 (—CH═), 134.5 (C-3), 146.2, 148.0, 158.0 (Ar q carbon), 174.5 (NHCOCH₃), (C-1, C-2 not observed); LRMS [C₂₃H₃₁NO₁₀] m/z (− ve ion mode): 480.1 [M-H]⁺, 439.1, 394.2, 277.0; HRMS (FAB): Calc. for C₂₃H₃₁N₁O₁₀Na₁ (+1): 504.184017. Found: m/z 504.185864.

### Example 19

### Methyl 5-acetamido-3-C-(3′-acetoxypropyl)-4,7,8,9-tetra-O-acetyl-2,6-anhydro-3,5-dideoxy-D-glycero-D-galacto-non-2-en-onate (11)

To a solution of the allyl derivative (6) (200 mg, 0.38 mmol) in dry THF (20 mL) under N₂, was added 9-BBN solution in THF (0.5 M) (1.54 mL, 0.77 mmol). The reaction mixture was stirred at 50° C. for 12 h. The crude boronic acid (10) was treated with hydrogen peroxide (2 mL) and aq. NaOH solution (0.2 mL, 1 N) at 0° C. and the reaction mixture was stirred at room temperature for 30 min. The reaction mixture is diluted with ethyl acetate and washed with aq. NaCl. The organic phase was dried (Na₂SO₄), filtered and evaporated under reduced pressure. The crude product was dissolved in dry acetonitrile under N₂ and to it was added acetic anhydride (1 mL) followed by DMAP (5 mg). The reaction mixture was stirred at room for 24 h after which it was concentrated under reduced pressure. The residue was dissolved in ethyl acetate, washed with aq. NaCl, dried (Na₂SO₄), filtered, and evaporated under reduced pressure. The crude product was purified by flash chromatography on silica to afford the title compound (11) as a white foam (20 mg, 9% over three steps).

R_{f} 0.5 (EtOAc); ¹H NMR (300 MHz, CDCl₃): δ 1.74 (m, 2H, —CH₂—), 1.88, 2.01, 2.02, 2.03, 2.08 (5s, 18H, NHCOCH₃, OCOCH₃×5), 2.25-2.31 (m, 1H, —CH₂—), 2.43-2.53 (m, 1H, —CH₂—), 3.74 (s, 3H, COOCH₃), 4.00-4.03 (m, 2H, —CH₂—OAc), 4.12 (dd, J=12.3, 6.9 Hz, 1H, H-9), 4.23 (dd, J=9.3, 3.6 Hz, 1H, H-6), 4.38 (ddd, J=16.8, 9.3 Hz, 1H, H-5), 4.61 (dd, J=12.0, 2.4 Hz, 1H, H-9′), 5.22-5.27 (m, 1H, H-8), 5.47 (dd, J=7.5, 2.7 Hz, 1H, H-7), 5.59 (d, J=7.8 Hz, 1H, H-4), 5.68 (d, J=9.3 Hz, 1H, NH); ¹³C NMR (75.5 MHz, CDCl₃): δ 20.7, 20.8, 20.9 (OCOCH₃×5, NHCOCH₃), 23.9 (—CH₂—), 27.6 (—CH₂—), 47.3 (C-5), 52.1 (COOCH₃), 62.0 (C-9), 63.8 (—CH₂—OAc), 67.4 (C-7), 70.5 (C-4), 70.9 (C-8), 76.0 (C-6), 121.4 (C-3), 141.3 (C-2), 162.2 (C-1), 170.1, 170.2, 170.6, 171.1, 171.2 (NHCOCH₃, OCOCH₃×5). LRMS (+ ve mode): m/z 596.2 [M+Na]⁺, 554, 514.

### Example 20

### 5-Acetamido-3-C-(3′-hydroxypropyl)-2,6-anhydro-5-dideoxy-D-glycero-D-galacto-non-2-enonic acid (12)

Compound (11) was deprotected according to the general procedure 5° C. for 16 h. The crude product was purified by reverse phase HPLC and then lyophilized to give title compound (12) as white solid (42%).

R_{f} 0.2 (EtOAc/MeOH/H₂O, 7:2:1); ¹H NMR (300 MHz, D₂O): δ 1.47-1.62 (m, 2H, —CH₂—), 1.91 (s, 3H, NHCOCH₃), 2.21-2.28 (m, 1H, —CH₂—), 2.42-2.52 (m, 1H, —CH₂—), 3.43-3.51 (m, 4H, H-7, H-9, —CH₂—OH), 3.68-3.74 (m, 2H, H-8, H-9′), 3.98-4.00 (m, 2H, H-5, H-6), 4.22 (dd, J=6.6, 2.4 Hz, 1H, H-4). LRMS (−ve mode): m/z 348.1 [M-1]⁺.

### Example 21

### Methyl 5-acetamido-4,7,8,9-tetra-O-acetyl-3,5-dideoxy-3-C-propyl-D-erythro-β-L-gluco-non-2-ulopyranosonate (13)

Allyl compound (3) was dissolved in methanol (4 mL) and to it added acetic acid (4 mL), followed by Pd/C (10%). The reaction flask was degassed using vacuum and then hydrogenation reaction was carried out using Parr apparatus under hydrogen (40 psi) at room temperature. The progress of reaction was monitored by TLC, after complete consumption of starting material reaction mixture was filtered through celite bed, residue was washed with methanol (3×10 mL) and combined organic phase was concentrated under reduced pressure to give alkane derivative. The crude product was purified by flash chromatography on silica gel to afford the title compound (13) as a white solid (95 mg, isolated yield 95%).

R_{f} 0.6 (EtOAc); ¹H NMR (300 MHz, CDCl₃): δ 0.75 (t, J=6.6 Hz, 3H, —CH₃), 1.10-1.19 (m, 4H, —CH₂CH₂—) 1.82 (s, 3H, NHCOCH₃), 1.95, 1.98, 2.03, 2.07 (4 s, 12H, OCOCH₃×4), 2.30-2.37 (m, 1H, H-3), 3.82 (s, 3H, COOCH₃), 3.94 (dd, J=12.3, 7.2 Hz, 1H, H-9), 4.11-4.17 (m, 2H, H-5, H-6), 4.34 (dd, J=12.6, 2.4 Hz, 1H, H-9′), 4.96 (t, J=10.5, 9.9 Hz, 1H, H-4), 5.10-5.15 (m, 1H, H-8), 5.27 (dd, J=6.3, 5.7 Hz, 1H, H-7), 6.05 (d, J=9.3 Hz, 1H, NH), 6.31 (bs, 1H, OH); ¹³C NMR (75.5 MHz, CDCl₃): δ 14.2 (—CH₃), 20.7, 20.7, 20.8, 20.9, (OCOCH₃×4), 22.9 (NHCOCH₃), 30.3 (—CH₂—CH₂—), 44.0 (C-3), 49.6 (C-5), 53.5 (COOCH₃) δ2.6 (C-9), 67.9 (C-7), 70.4 (C-6), 70.9 (C-8), 74.4 (C-4), 96.9 (C-2), 170.0, 170.3, 170.6, 170.7, 170.8, 171.6 (NHCOCH₃, OCOCH₃×4, COOCH₂). C₂₃H₃₅NO₁₃: LRMS (− ve ion mode): m/z 531.5 [M-H]⁺.

### Example 22

### Methyl 5-acetamido-4,7,8,9-tetra-O-acetyl-2,6-anhydro-3,5-dideoxy-3-C-propyl-D-glycero-D-galacto-non-2-enonate (15)

To a solution of allyl derivative (13) (95 mg, 0.17 mmol) in acetyl chloride (10 mL) at 0° C. was added dry methanol (0.2 mL). The reaction mixture was stirred at room temperature in a sealed (glass-stoppered) round bottom flask for 48 h. The reaction mixture was concentrated under reduced pressure and azeotroped with toluene (3×5 mL) to yield chloride (14) as an off-white foam. The crude chloride (98 mg) was taken up in dry dichloromethane (5 mL), to which DBU (92 microL, 0.61 mmol) was added. The reaction was left to stir at room temperature under N₂ for 16 h. The reaction mixture was evaporated to dryness, taken up in chloroform and washed with saturated aq. NH₄Cl, H₂O, and satd aq. NaCl. The organic phase was dried (Na₂SO₄), filtered and evaporated under reduced pressure, and the residue purified by flash chromatography on silica gel to afford the title compound (15) as a white solid (75 mg, isolated yield 83%).

R_{f} 0.6 (EtOAc); ¹H NMR (300 MHz, CDCl₃): δ 0.87 (t, J=7.2 Hz, 3H, —CH₃), 1.31-1.53 (m, 2H, —CH₂—), 1.88 (s, 3H, NHCOCH₃), 2.0.2, 2.04, 2.07, 2.09 (4 s, 12H, OCOCH₃×4), 2.09-2.20 (m, 1H, —CH₂—), 2.40-2.50 (m, 1H, —CH₂—), 3.75 3.82 (s, 3H, COOCH₃), 4.10 (dd, J=6.9, 2.4 Hz, 1H, H-9), 4.19 (dd, J=9.6, 3.3 Hz, 1H, H-6), 4.34-4.43 (m, 1H, H-5), 4.61 (dd, J=12.3, 2.7 Hz, 1H, H-9′), 5.22-5.27 (m, 1H, H-8), 5.41-5.47 (m, 2H, H-7, NH [D₂O exchanged]), 5.59 (d, J=8.1 Hz, 1H, H-4); ¹³C NMR (75.5 MHz, CDCl₃): δ 14.0 (—CH₃), 20.7, 20.8 (OCOCH₃×4), 22.0 (—CH₂—), 23.1 (NHCOCH₃), 29.1 (—CH₂—), 47.6 (C-5), 52.1 (COOCH₂), 62.0 (C-9), 67.4 (C-4), 70.6 (C-7), 70.9 (C-8), 76.1 (C-6), 122.7 (C-3), 162.3 (C-2), 170.1, 170.5 (NHCOCH₂, OCOCH₃×4, COOCH₃). C₂₃H₃₃NO₁₂: LRMS (+ ve ion mode): m/z 537.8 [M+Na]⁺455.8 (M-COOCH₃); LRMS (− ve ion mode): m/z 513.6 [M-H]⁺.

### Example 23

### 5-Acetamido-2,6-anhydro-3,5-dideoxy-3-C-propyl-D-glycero-D-galacto-non-2-enonic acid (16)

Compound (15) (65 mg, 0.12 mmol) was dissolved in anhydrous methanol and solution was cooled to 0° C. using ice bath. Sodium methoxide (1M) solution was added to reaction mixture and after 10 mins reaction mixture was brought to room temperature. The reaction mixture was stirred at rt for 5 h. The progress of reaction was monitored by TLC analysis. The reaction mixture was acidified to pH 6 using Amberlite® IR-120 (H⁺) resin and the solution was filtered through cotton plug. The resin was washed with water and the combined filtrate was evaporated to dryness to give the deacetylated product as an off white solid (43 mg, isolated yield 100%). The deacetylated compound was deprotected according to the general procedure at rt for 3 h. The crude product was purified by reverse phase HPLC and then lyophilized to give title compound (16) as white solid (29 mg, isolated yield 71%).

R_{f} 0.2 (EtOAc/MeOH/H₂O, 7:2:1); ¹H NMR (300 MHz, D₂O): δ 0.87 (t, J=7.5, 7.2 Hz, 3H, —CH₃), 1.26-1.59 (m, 2H, —CH₂—), 2.05 (s, NHCOCH₃), 2.31 (m, 1H, —CH₂—), 2.50 (m, 1H, CH₂—), 3.59-3.66 (m, 2H, H-7, H-9), 3.84-3.87 (m, 2H, H-8, H-9′), 4.12-4.14 (m, 2H, H-5, H-6), 4.35 (dd, J=6.3, 2.7 Hz, 1H, H-4); ¹³C NMR (75.5 MHz, CDCl₃): δ 13.0 (—CH₃), 21.1 (—CH₂—), 22.0 (NHCOCH₃), 27.9 (—CH₂—), 50.5 (C-5), 62.9 (C-9), 68.0 (C-7), 68.8 (C-4), 69.8 (C-8), 75.3 (C-6), 162.3 (C-1), 174.6 (NHCOCH₃), (C-2 and C-3 not observed); C₁₄H₂₃NO₉: LRMS (− ve ion mode): m/z 331.8 [M-H]⁺.

### Example 24

### Methyl 5-acetamido-4,7,8,9-tetra-O-acetyl-2,6-anhydro-3,5-dideoxy-3-C-propenyl-D-glycero-D-galacto-non-2-enonate (17)

Anhydrous MeOH (2 mL, 0.06 mol) was slowly added dropwise to solution of glycosyl acetate (4) (128 mg, 0.22 mmol) in AcBr (10 mL, 0.14 mol) with cooling in an ice-water bath. (Caution! This reaction is exothermic and rapid addition of methanol can result in violent release of HCl gas). The reaction mixture was then stirred at room temperature in a sealed (glass-stoppered) round bottom flask for 8 h. The reaction mixture was concentrated under reduced pressure and azeotroped with toluene (3×20 mL) to yield the glycosyl bromide as an off-white foam. The crude bromide was taken up in dry dichloromethane (5 mL), to which DBU (99 microL, 0.66 mmol, 3 mole equiv.) was added, and the reaction was left to stir at room temperature under N₂ for 2 h. The reaction mixture was evaporated to dryness, taken up in chloroform and washed successively with satd aq. NH₄Cl, H₂O, and satd aq. NaCl. The organic phase was dried (anhydrous Na₂SO₄), filtered and evaporated under reduced pressure, and the residue purified by flash chromatography on silica gel (Acetone/Hexanes 30:70) to afford the title compound (17) as a white solid (64 mg, isolated yield 56%, over 2 steps).

R_{f} 0.7 (EtOAc); ¹H NMR (300 MHz, CDCl₃): δ 1.75 (dd, J=6.6, 1.5 Hz, 3H, —CH₃), 1.90 (NHCOCH₃), 2.02, 2.03, 2.04, 2.08 (4×s, 12H, OCOCH₃×4), 3.77 (s, 3H, COOCH₃), 4.12-4.20 (m, 2H, H-6, H-9), 4.43 (ddd, J=9.3, 8.1, 6.3 Hz, 1H, H-5), 4.50 (dd, J=12.3, 3.0 Hz, 1H, H-9′), 5.24 (m, 1H, H-8), 5.50-5.62 (m, 2H, H-7, ═CH—), 5.66 (d, J=9.3 Hz, 1H, NH), 5.79 (d, J=6.0 Hz, 1H, H-4), 6.88 (dd, J=16.2, 1.5 Hz, 1H, —CH═); ¹³C NMR (75.5 MHz, CDCl₃): δ 19.2 (—CH₃), 20.7, 20.8 (OCOCH₃×4), 23.1 (NHCOCH₃), 48.0 (C-5), 52.3 (COOCH₃), 61.8 (C-9), 67.0 (C-7), 67.3 (C-4), 70.3 (C-8), 76.0 (C-6), 119.5 (C-3), 123.8 (═CH—), 128.8 (═CH—), 140.8 (C-2), 162.3 (C-1), 169.9, 170.1, 170.6, 170.9 (NHCOCH₃, OCOCH₃×4). LRMS [C₂₃H₃₁NO₁₂] (+ ve ion mode) m/z: 536.1 [M+Na]⁺.

### Example 25

### Methyl 5-acetamido-2,6-anhydro-3,5-dideoxy-3-C-(prop-2′-enyl)-D-glycero-D-galacto-non-2-enonate (18)

Compound (6) (127 mg, 0.24 mmol) was dissolved in anhydrous methanol and solution was cooled to 0° C. using ice bath. Sodium methoxide solution (1M) was added to reaction mixture and after 10 mins reaction mixture was brought to room temperature. The reaction mixture was stirred at rt for 4 h. The progress of reaction was monitored by TLC analysis. The reaction mixture was acidified to pH 6 using Amberlite® IR-120 (H⁺) resin and the solution was filtered through cotton plug. The resin was washed with water and combined filtrates was evaporated to dryness to give deacetylated compound (18) as an off white solid [TLC (EtOac/MeOH, 4:1): R_{f} 0.2]. The crude product was used without further purification in the subsequent 8,9-O-isopropylidene derivative. A crude yield of 98% was obtained.

### Example 26

### Methyl 5-acetamido-2,6-anhydro-3,5-dideoxy-8,9-O-isopropylidene-3-C-(prop-2′-enyl)-D-glycero-D-galacto-non-2-enonate (19)

Compound (18) was dissolved in mixture of dry acetone (2 mL) and 2,2-dimethoxypropane (1 mL) at room temperature under an atmosphere of argon. This was followed by the addition of Amberlite® IR-120 (H⁺) resin and the reaction was stirred at rt for 16 h. After the removal of the resin by filtration, and evaporation of the solvent, subsequent treatment with dry NEt₃ and resuspension of the resultant residue in DCM yielded the product (19) as a white precipitate in quantitative yield (60 mg, 71%).

R_{f} 0.3 (EtOAc): LRMS [C₁₈H₂₇NO₈] m/z (+ ve ion mode): 408.1 [M+Na]⁺; (− ve ion mode): 384.1 [m−1]⁺.

### Example 27

### Methyl 5-acetamido-2,6-anhydro-3,5-dideoxy-4-O-ethyl-8,9-O-isopropylidene-3-C-(prop-2′-enyl)-D-glycero-D-galacto-non-2-enonate (20)

To a solution of compound (19) (50 mg, 0.12 mmol) in dry DMF was added ethyl iodide (20 mL, 0.25 mmol). The reaction mixture was stirred at 0° C. for 10 minutes and then sodium hydride (4 mg, 0.16 mmol) was added. The reaction mixture was stirred at 0° C. for 2 h. The progress of reaction was monitored by TLC analysis. The reaction mixture was then quenched with 0.1 mL of dry MeOH and, after a workup consisting of evaporation of DMF and aqueous extraction, the crude product was chromatographed using 5:1 EtOAc/hexanes as eluent to give the desired product (20) as an off-white foam (25 mg, 47%).

R_{f} 0.7 (EtOAc); LRMS [C₂₀H₃₁NO₈] m/z ve ion mode): 436.1 [M+Na]⁺; (− ve ion mode): 412.1 [m−1]⁺.

### Example 28

### 5-Acetamido-2,6-anhydro-3,5-dideoxy-4-O-ethyl-3-C-(prop-1-enyl)-D-glycero-D-galacto-non-2-enonic acid (22)

The deprotection steps involved the initial removal of the isopropylidene group protecting the C-8 and C-9 hydroxyl groups followed by the de-esterification of the C-1 carboxylic acid. De-isopropylidination of (20) was carried-out by the use of 80% AcOH at 80° C. for 1 hr. After evaporation of the AcOH, de-esterification of (21) was carried-out according to the general procedure at 0° C.-rt, 12 h. The crude product was purified by reverse phase HPLC and then lyophilized to give the title compound (22) as a white solid (18 mg, 83%).

R_{f}0.2 (EtOAc/MeOH/H₂O, 7:2.5:0.5); ¹H NMR (300 MHz, D₂O): δ 1.12 (t, J=7.2, 6.9 Hz, 3H, (—CH₃—), 1.99 (s, 3H, NHCOCH₃), 2.86 (dd, J=15.0, 7.2 Hz, 1H, —CH₂—), 3.26 (dd, J=15.1, 5.2 Hz, 1H, —CH₂—), 3.53-3.68 (s, 4H, H-9, —CH₂—CH₃), 3.78-3.83 (m, 2H, H-8, H-9′), 4.11-4.21 (m, 2H, H-5, H-6), 4.30 (dd, J=8.4, 1.8 Hz, 1H, H-4), 5.01-5.12 (m, 2H, —CH₂—), 5.80 (m, 1H, —CH═); ¹³C NMR 20, (75.5 MHz, CDCl₃): δ 15.1 (—CH₃), 22.4 (NHCOCH₃), 31.0 (—CH₂—), 47.7 (C-5), 63.8 (C-9), 65.2 (—CH₂—CH₃), 68.5 (C-7), 70.3 (C-4), 76.1 (C-8), 76.8 (C-6), 116.4 (—CH₂—), 116.8 (C-3), 136.4 (—CH═), 144.9 (C-2), (C-1 and NHCOCH₃ not observed). LRMS [C₁₆H₂₅NO₈]: m/z (− ve ion mode): 358.1 [M-H]⁺, 314.1, 248.7, 207.9, 177.9; HRMS (FAB): Calc for C₁₆H₂₅N₁O₈Na₁ (+1): 382.147238. Found: m/z 382.147911.

### Example 29

### 2-Methyl-(methyl 7,8,9-tri-O-acetyl-2,6-anhydro-3,5-dideoxy-3-C-(prop-2′-enyl)-D-glycero-D-talo-non-2-enonate)-[4,5-d]-2-oxazoline (23)

To a solution of allyl derivative (4) (100 mg, 0.174 mmol) in anhydrous dichloromethane (10 mL) under N₂, was added boron trifluoride diethyl etherate (217 microL, 1.74 mmol) and the reaction was stirred at rt for 48 h. The progress of reaction was monitored by TLC analysis. The mixture was then slowly poured on to a stirring ice (1.5 g)-water (4.5 mL) mixture containing EtOAc (25 mL) and Na₂CO₃ (850 mg). The aqueous layer washed with a saturated NaCl solution (3×5 mL), and subsequently dried over anhydrous Na₂SO₄. Evaporation of the organic filtrate afforded crude product which was purified by column chromatography on silica (Acetone-Hexane, 30:70) to yield the title compound (23) as a white foam (48 Mg, 61%).

R_{f} 0.7 (EtOAc); ¹H NMR (300 MHz, CDCl₃): δ 2.01, 2.04, 2.06, 2.1 (4×s, 12H, OCOCH₃×3 and oxazoline Me), 3.00 (dd, J=14.1, 8.4 Hz, 1H, —CH₂—), 3.29 (dd, J=10.5, 2.4 Hz, 1H, H-6), 3.68 (dd, J=14.4, 6.6 Hz, 1H, —CH₂—), 3.77 (s, 3H, COOCH₃), 3.93 (ddd, J=9.9, 9.0, 2.7 Hz, 1H, H-5), 420 (dd, J=12.6, 6.3 Hz, 1H, H-9), 4.62 (dd, J=12.6, 2.4 Hz, 1H, H-9′), 4.77 (d, J=9.0 Hz, 1H, H-4), 5.06-5.18 (m, 2H, ═CH₂), 5.41 (ddd, J=6.3, 2.7, 2.4 Hz, 1H, H-8), 5.62 (dd, J=6.0, 2.4 Hz, 1H, H-7), 5.85 (m, 1H, —CH═); ¹³C NMR (75.5 MHz, CDCl₃): δ 14.2 (oxazoline —CH₃), 20.6, 20.8, 20.9 (OCOCH₃×3), 32.8 (—CH₂—), 52.2 (COOCH₃), 62.0 (C-9), 62.2 (C-5), 68.6 (C-7), 70.3 (C-8), 74.7 (C-4), 76.4 (C-6), 117 (═CH₂), 121.5 (C-3), 134.6 (═CH—), 142.2 (C-2), 162.3 (C-1), 166.8 (oxazoline CO), 169.6, 169.8, 170.7 (OCOCH₃×3). C₂₁H₂₇NO₁₀: LRMS (+ ve ion mode): m/z 476.4 [M-H]⁺.

### Example 30

### Methyl 5-acetamido-7,8,9-tri-O-acetyl-2,6-anhydro-4-azido-3-C-(prop-2′-enyl)-3,4,5-trideoxy-D-glycero-D-galacto-non-2-enonate (24)

To a solution of allyl derivative (6) (375 mg, 0.73 mmol) in anhydrous dichloromethane (10 mL) under N₂, was added anhydrous methanol (23 mL, 0.74 mmol) followed by boron trifluoride diethyl etherate (916 microL, 7.3 mmol) and the reaction mixture was stirred at rt for 20 h. The mixture was then slowly poured on to a stirring ice (1.5 g)-water (4.5 mL) mixture containing EtOAc (25 mL) and Na₂CO₃ (850 mg). The aqueous layer washed with a saturated NaCl solution (3×5 mL), and subsequently dried over anhydrous Na₂SO₄. Evaporation of the organic filtrate afforded crude product (23) (313 mg, 75%), which was used without further purification in the subsequent reaction. To a solution of oxazoline derivative (23) (313 mg, 0.69 mmol) in anhydrous tert-butanol (4 mL) under N₂, was added azidotrimethylsilane (764 microL, 5.78 mmol) and the reaction was stirred at 80° C. for 24 h. The reaction mixture was diluted with ethyl acetate (50 mL) and the organic layer was washed with hydrochloric acid (0.1 N, 4 mL) and water (2×mL). The combined aqueous layer was extracted with ethyl acetate (2×5 mL). The combined organic extracts were then dried (anhydrous Na₂SO₄), and evaporated under reduced pressure to give crude product which was purified by column chromatography on silica (Acetone-Hexane, 30:70) to yield the title compound (24) as a white foam (105 mg, 31%).

R_{f} 0.8 (EtOAc); ¹H NMR (300 MHz, CDCl₃): δ 1.84 (dd, J=6.6, 1.5 Hz, 3H, —CH₃), 1.97, 2.02, 2.03, 2.12 (4 s, 12H, NHCOCH₃, OCOCH₃×3), 3.77 (s, 3H, COOCH₃), 4.10-4.17 (m, 2 H, H-5, H-9), 4.29 (dd, J=8.4, 3.9 Hz, 1H, H-6), 4.37 (d, J=6.9 Hz, 1H, H-4), 4.51 (dd, J=12.3, 3.0 Hz, 1H, H-9′), 5.30 (m, 1H, H-8), 5.46 (m, 1H, H-7), 5.82 (d, J=8.4 Hz, 1H, NH) [D₂O exchanged], 5.95 (m, 1H, ═CH—), 6.99 (d, J=15.9 Hz, 1H, —CH═); ¹³C NMR (75.5 MHz, CDCl₃): δ 19.2 (—CH₃), 20.7, 20.8 (OCOCH₃×3), 23.4 (NHCOCH₃), 49.9 (C-5), 52.3 (COOCH₃), 58.4 (C-4), 61.7 (C-9), 67.6 (C-7), 70.0 (C-8), 75.4 (C-6), 119.7 (C-3), 123.9 (—CH═), 130.4 (═CH—CH₃), 140.8 (C-2), 162.4 (C-1), 169.8, 170.2, 170.3, 170.6 (NHCOCH₃, OCOCH₃×3). C₂₁H₂₈NO₁₀: LRMS (+ ve ion mode): m/z 518.8 [M-H]⁺, 490.9, 430.8, 370.8, 306.8, 257.9.

### Example 31

### Methyl 5-Acetamido-4,7,8,9-tetra-O-acetyl-3,5-dideoxy-3-bromo-D-erythro-β-L-manno-non-2-ulopyranosonate (26)

To a solution of 2,3-dibromide (25) (330 mg, 0.52 mmol) [prepared from (1) according to the method of Okamoto et al., 1987] in anhydrous dichloromethane (10 mL) at 0° C., was added silver carbonate (215 mg, 0.78 mmol) and silver perchlorate (162 mg, 0.78 mmol). The mixture was stirred, protected from light, for 15 min at 0° C. and a further 30 min at room temperature. The mixture was filtered through Celite and the filtrate concentrated under vacuum. The crude product was, purified by chromatography on silica (hexane/acetone 6:4) to give the title compound (26) (Okamoto et al., 1987) as a white solid (276 mg, 0.48 mmol, 93%).

R_{f} 0.36 (EtOAc); ¹H NMR (300 MHz, CDCl₃): δ 1.90 (NHCOCH₃), 2.03, 2.08, 2.09, 2.16 (4s, 12H, OCOCH₃), 3.86 (s, 3H, COOCH₃), 4.12 (dd, 1H, J_{9,8}=8.2 Hz, J_{9,9}=12.5 Hz, H-9), 4.38-4.43 (m, 2H, H-5, H-6), 4.59 (d, 1H, J_{3,4}=3.7 Hz, H-3), 4.94 (dd, 1H, J_{9,8}=2.2 Hz, J_{9′,9}=12.5 Hz, H-9′), 5.25 (m, 1H, H-8), 5.36 (dd, 1H, J_{7,6}=1.5 Hz, J_{7,8}=3.6 Hz, H-7), 5.41 (dd, 1H, J_{4,3}=3.7 Hz, J_{4,5}=10.0 Hz, H-4), 5.95 (s, 1H, OH), 6.10 (d, 1H, J_{NH,5}=6.02 Hz, NH); LRMS (ESI): m/z 592.4, 594.4 [M+Na)⁺].

### Example 32

### Methyl 5-Acetamido-4,7,8,9-tetra-O-acetyl-2,3-anhydro-5-dideoxy-D-erythro-β-L-gluco-non-2-ulopyranosonate (27)

According to the method of Okamoto et al. (Okamoto et al., 1987), a solution of (26) (426 mg, 0.75 mmol) in dry acetonitrile (4 mL) under N₂ at room temperature, was treated with DBU (140 microL, 0.90 mmol). The mixture was stirred for 15 min at room temperature and then the solution was applied to a silica-gel column and chromatographed (toluene/acetone 3:2) to give the title compound (27) (Okamoto et al., 1987) (315 mg, 85% yield) as a white foam.

R_{f} 0.53 (EtOAc); ¹H NMR (300 MHz, CDCl₃): δ 1.91 (s, 3H, NH COCH₃), 2.03, 2.05, 2.11, 2.12 (4s, 12H, OCOCH₃), 3.59 (z, 1H, H-3), 3.83 (s, 3H, COOCH₃), 4.06 (dd, 1H, J_{6,7}=4.5 Hz, J_{6,5}=8.4 Hz, H-6), 4.15 (dd, 1H, J_{9,8}=6.9 Hz, J_{9,9′}=12.5 Hz, H-9), 4.24 (m, 1H, H-5), 4.51 (dd, 1H, J_{9′,8}=3.0 Hz, J_{9′,9}=12.5 Hz, H-9′), 5.19 (d, 1H, J_{4,5}=7.5 Hz, H-4), 5.25 (m, 1H, H-8), 5.40 (dd, 1H, J_{7,6}=3.7 Hz, J_{7,8}=5.1 Hz, H-7), 5.51 (d, 1H, J_{NH,5}=9.9 Hz, NH): LRMS (ESI): m/z 513.4 [M+Na)⁺].

### Example 33

### Methyl (4′-methoxybenzyl-5-Acetamido-4,7,8,9-tetra-O-acetyl-5-dideoxy-D-erythro-α-L-gluco-non-2-ulopyranoside)onate (28)

To a solution of (27) (560 mg, 1.14 mmol) in dry dichloroethane (5 mL) at 0° C. under N₂, was added p-methoxybenzyl alcohol (3 mL) and then camphor-sulfonic acid (catalytic). After stirring for 15 min at 0° C., the reaction was let warm to room temperature for 1 h. The chlorinated solvent was remove under vacuum and the residual oily solution was purified by flash chromatography (EtOAc/dichloromethane gradient 1:1 to 8:2) to yield the (28) (580 mg, 81%) as a white solid.

R_{f} 0.39 (EtOAc); ¹H NMR (300 MHz, CDCl₃): δ 1.86 (s, 1H, NHCOCH₃) 2.01, 2.03, 2.06 2.08 (4s, 12H, OCOCH₃), 2.69 (d, 1H, J_{OH,3}=4.8 Hz, OH), 3.79 (z, 3H, OCH₃), 3.80 (s, 3H, COOCH₃), 3.82 (dd, 1H, J_{3,4}=9.6 Hz, J_{OH,3}=4.8 Hz, H-3), 4.04 (dd, 1H, J_{5,5}=6.0 Hz, J_{9,9′}=12.6 Hz, H-9), 4.20-4.28 (m, 2H, H-5, H-9′), 4.51 (d, 1H, CH₂PMB), 4.58 (dd, 1H, J_{6,7}=2.1 Hz, J_{6,5}=10.8 Hz, H-6), 4.78 (d, 1H, CH₂PMB), 5.13 (dd, 1H, J_{4,5}=J_{4,3}=10.2 Hz, H-4), 5.26 (dd, 1H, J_{7,6}=1.8 Hz, J_{7,8}=8.4 Hz, H-7), 5.32-5.41 (m, 2H, H-8, NH), 6.86 (d, 2H, Ph), 7.31 (d, 2H, Ph); LRMS (ESI): m/z 650.2 [M+Na)⁺].

### Example 34

### Methyl (4′-methoxybenzyl-5-Acetamido-4,7,8,9-tetra-O-acetyl-5-dideoxy-3-O-ethyl-D-erythro-α-L-gluco-non-2-ulopyranoside)onate (29)

Compound (28) (0.882 g, 1.41 mmol) was dissolved in anhydrous DMF (40 mL) under N₂ at room temperature, and activated MS 4 \dot{A}, (1 g) were added. After stirring for 1 h, ethyl iodide (0.57 mL, 7.03 mmol), freshly prepared Ag₂O (1.625 g, 7.03 mmol) [Campaigne and LeSuer, Organic Syntheses Coll. (1963), 4, 919], and tetrabutylammonium iodide (260 mg, 0.705 mmol) were added. After completion of addition, the reaction mixture was stirred, protected from light, for 16 h at room temperature. The solution was filtered through Celite and the filtrate concentrated under reduced pressure. The residue was purified by chromatography on silica (EtOAc/dichloromethane 6:4) to yield (29) (444 mg, 48%) as a white solid foam.

R_{f}=0.73 (EtOAc); ¹H NMR (300 MHz, CDCl₃): δ 1.05 (t, 3H, OCH₂CH₃), 2.03 (s, 1H, NHCOCH₃), 2.03, 2.04, 2.06, 2.10 (4s, 12H, COCH₃), 3.56 (m, 2H, H-3, OCH₂CH₃), 3.78 (s, 3H, OCH₃), 3.80 (s, 3H, COOCH₃), 3.80-3.84 (m, 1H, OCH₂CH₃), 4.03 (dd, 1H, J_{9,8}=6.0 Hz, J_{9,9′}=12.6 Hz, H-9), 4.20-4.35 (m, 2H, H-5, H-9′), 4.52 (d, 1H, CH₂PMB), 4.68-4.78 (m, 2H, H-6, CH₂PMB), 5.13 (dd, 1H, J_{4,5}=J_{4,3}=9.0 Hz, H-4), 5.25 (dd, 1H, J_{7,6}=1.8 Hz, J_{7,8}=8.4 Hz, H-7), 5.33 (m, 1H, H-8), 5.43 (d, 1H, J_{NH,5}=10.2 Hz, NH), 6.86 (d, 2H, J=11.4 Hz, PMB), 7.31 (d, 2H, J=11.4 Hz, PMB); LRMS (ESI): m/s 677.8 [M+Na)⁺].

### Example 35

### Methyl 5-acetamido-4,7,8,9-tetra-O-acetyl-3,5-dideoxy-3-O-ethyl-D-erythro-β-L-gluco-non-2-ulopyranosonate (30)

To a solution of (29) (300 mg, 0.46 mmol) in a mixture of dichloromethane (45 mL) and H₂O (5 mL), was added DDQ (229 mg, 1.01 mmol). The reaction was stirred for 54 h at room temperature. The reaction mixture was then washed with saturated NaHCO₃, brine, dried (Na₂SO₉), filtered, and the filtrate concentrated under vacuum. The residue was purified by chromatography on silica (EtOAc/dichloromethane 7:3) to yield (30) (193 mg, 79%).

R_{f}=0.56 (EtOAc); ¹H NMR (300 MHz, CDCl₃): δ 1.04 (t, 3H, OCH₂CH₃), 1.85 (s, 1H, NHCOCH₃), 1.98, 2.03, 2.05, 2.10 (4s, 12H, COCH₃), 3.58 (m, 2H, OCH₂CH₃), 3.86-3.99 (m, 2 H, H-3, H-9), 3.8 (s, 3H, OCH₃), 4.01-4.43 (m, 3H, H-5, H-6, H-9′), 5.11-5.22 (m, 2H, H-4, H-8), 5.58-5.32 (dd, 1 H, J_{7,6}=1.8 Hz, J_{7,8}=8.4 Hz, H-7), 5.95 (d, 1H, J_{NH,5}=9.9 Hz, NH); LRMS (ESI): m/z 557.9 [M+Na)⁺].

### Example 36

### Methyl 5-acetamido-2,4,7,8,9-penta-O-acetyl-3,5-dideoxy-3-O-ethyl-D-erythro-β-L-gluco-non-2-ulopyranosonate (31)

Compound (30) (160 mg, 0.30 mmol) was dissolved in dry pyridine (3 mL) and acetic anhydride (2 mL) and DMAP (catalytic amount) were added to the reaction mixture. After stirring for 16 h the reaction was concentrated under reduced pressure and the residue was purified by chromatography on silica (EtOAc/dichloromethane 7:3) yielding (31) (173 mg, 95%).

R_{f}=0.43 (EtOAc); ¹H NMR (300 MHz, CDCl₃): δ 1.05 (t, 3H, OCH₂CH₃), 1.84 (s, 1H, NHCOCH₃), 2.00, 2.02, 2.08, 2.14, 2.19 (5s, 15H, COCH₃), 3.52-3.57 (m, 2H, OCH₂CH₃), 3.67 (d, 1H, J_{3,4}=9.6 Hz, H-3), 3.80 (s, 3H, COOCH₃), 3.95 (dd, 1H, J_{6,7}=2.4 Hz, J_{6,5}=10.8 Hz, H-6), 4.06-4.29 (m, 2H, H-5, H-9), 4.48 (dd, 1H, J_{9′,8}=3.0 Hz, J_{9′,9}=12.3 Hz, H-9′), 4.96 (m, 1H, H-8), 5.16 (t, 1H, J_{4,3}=J_{4,5}=9.9 Hz, H-4), 5.31 (dd, 1H, J_{7,6}=2.1 Hz, J_{7,4}=4.2 Hz, H-7), 5.55 (d, 1H, J_{NH,5}=9.9 Hz, NH); LRMS (ESI): m/z 599.8 [M+Na)⁺].

### Example 37

### Methyl 5-acetamido-4,7,8,9-tetra-O-acetyl-2,6-anhydro-3,5-dideoxy-3-O-ethyl-D-glycero-D-galacto-non-2-enonate (33)

Compound (31) (74 mg 0.128 mmol) was dissolve in anhydrous 1,2-dichloroethane (1 mL) under N₂ and the solution cooled to 0° C., when HBr—ACOH (33%, 2 mL) was added dropwise. The reaction was stirred for 1 h at 0° C., and then for another 2 h at room temperature. The solution was diluted with anhydrous toluene and evaporated under reduced pressure. Evaporation with toluene was repeated a further 2 times to give the crude glycosyl bromide (32) as a yellow solid. Compound (32) was used without purification for the elimination reaction. Crude (32) (0.128 mmol) was dissolved in 1,2-dichloroethane (2 mL) under N₂ and the solution cooled to 0° C., when DBU (75 microL, 0.480 mmol) was added. The reaction was stirred overnight at room temperature then concentrated under reduced pressure. The residue was dissolved in EtOAc and washed with a saturated aqueous solution of NH₄Cl, water and brine, dried (Na₂SO₄), filtered and the filtrate concentrated under vacuum. The residue was purified by chromatography on silica (EtOAc/dichloromethane 65:35) to give compound (33) (37 mg, 56%) as a white foam. Unreacted (31) (13 mg, 17%) was also recovered.

R_{f} 0.65 (EtOAc); ¹H NMR (300 MHz, CDCl₃): δ 1.21 (t, 3H, OCH₂CH₃), 1.94 (s, 1H, NHCOCH₃), 2.06, 2.08, 2.10, 2.14 (4s, 12H, COCH₃), 3.71-3.76 (m, 1H, OCH₂CH₃), 3.75 (s, 3H, COOCH₃), 3.92-3.97 (m, 1H, OCH₂CH₃), 4.08-4.18 (m, 2H, H-6, H-9), 4.38 (m, 1H, H-5), 4.58 (dd, 1H, J_{9′,8}=3.0 Hz, J_{9,9′}=12.3 Hz, H-9′), 5.24 (m, 1H, H-8), 5.46 (m, 1 H, H-7), 5.70 (d, 1H, 7.2 Hz, H-4), 5.93 (d, 1H, J_{NH,5}=9.3 Hz, NH); ¹³C NMR (75.5 MHz, CDCl₃): δ 15.3 (OCH₂CH₃), 20.72, 20.79, 20.86, 20.93 (4×OCOCH₃), 23.07 (NHCOCH₃), 47.87 (C-5), 52.14 (COOCH₃), 61.96 (C-9), 67.25 (C-7), 68.65 (C-4), 70.11, (OCH₂CH₃), 70.92 (C-8), 76.33 (C-6), 136.76 (C-2), 142.85 (C-3), 169.57-170.56 (5×COCH₃); LRMS (ESI): m/z 539.8 [M+NA)⁺].

### Example 38

### 5-Acetamido-2,6-anhydro-3,5-dideoxy-3-O-ethyl-D-glycero-D-galacto-non-2-enonic acid (34)

Compound (33) was deprotected according to the general procedure at 5° C. for 12 h (19 mg, 88%).

¹H NMR (300 MHz, D₂O); δ 1.06 (t, 3H, OCH₂CH₃), 1.84 (s, 1H, NHCOCH₃), 3.36-3.44 (m, 2H, H-7, H-9), 3.62-3.76 (m, 3H, OCH₂CH₃H-8, H-9), 3.85-4.02 (m, 3H, OCH₂CH₃H-5, H-6), 4.38 (m, 1H, H-4); ¹³C NMR (75.5 MHz, D₂O): δ 14.13 (OCH₂CH₃), 21.94 (NHCOCH₃), 50.41 (C-5), 62.85 (C-9), 66.92 (C-4), 67.86 (C-7), 68.46 (OCH₂CH₃), 69.94 (C-8), 75.46 (C-6), 143.09 (C-3), 165.92 (C-1), 174.45 (NHCOCH₃), (C-2 not observed); LRMS (ESI): m/z (M-1)⁺: 334.4.

### Example 39

### Methyl (4′-methoxybenzyl-5-Acetamido-4,7,8,9-tetra-O-acetyl-5-deoxy-3-O-acetonitrile-D-erythro-α-L-gluco-non-2-ulopyranoside)onate (35)

Compound (28) (578 mg, 0.92 mmol) was dissolved in anhydrous dichloromethane (17 mL) under N₂ at room temperature and activated MS 4 \dot{A} (4 g) were added followed by bromoacetonitrile (245 microL, 3.68 mmol). After stirring for 1 h freshly prepared Ag₂O (854 mg, 3.68 mmol), and THAI (340 mg, 0.92 mmol) were added. After complete addition, the reaction mixture was stirred, protected from light, for 16 h at room temperature. The solution was filtered through Celite and the filtrate concentrated under reduced pressure. The residue was purified by chromatography on silica (hexane/acetone 6:4) to yield (35) (485 mg, 79%) as a white solid foam.

R_{f}=0.68 (EtOAc); ¹H NMR (300 MHz, CDCl₃): δ 1.86 (s, 3H, NH COCH₃), 2.00, 2.02, 2.07, 2.08, 2.13 (5s, 15H, COCH₃), 3.67 (d, 1H, H-3, J_{3,4} 10.6 Hz), 3.80 (s, 3H, OCH₃), 3.82 (s, 3H, COOCH₃), 4.03 (dd, 1H, J_{9,8}=6.0 Hz, J_{9,9′}=12.3 Hz, H-9), 4.21 (dd, 1H, J_{9′,8}=2.4 Hz, H_{9′,9}=12.3 Hz, H-9′), 4.29 (m, 1H, H-5), 4.43 (d, 2H, CH₂CN), 4.51 (d, 1H, CH₂PMB), 4.68-4.71-4.79 (m, 2H, H-6, CH₂PMB), 5.13 (dd, 1H, J_{4,5}=J_{4,3}=10.6 Hz, H-4), 5.26 (dd, 1H, J_{7,6}=2.1 Hz, J_{7,8}=9.0 Hz, H-7), 5.34-5.39 (m, 2H, H-8, NH), 6.86 (d, 2H, J=11.4 Hz, PMB), 7.29 (d, 2H, J=11.4 Hz, PMB); LRMS (ESI): m/z 688.9 [M+Na)⁺].

### Example 40

### Methyl 5-acetamido-4,7,8,9-tetra-O-acetyl-3,5-dodeoxy-3-O-acetonitrile-D-erythro-β-L-gluco-non-2-ulopyranosonate (36)

To a solution of (35) (485 mg, 0.73 mmol) in a mixture of dichloromethane (30 mL) and H₂O (2 mL) was added

DM (497 mg, 2.19 mmol). The reaction was stirred for 54 h at room temperature. The reaction mixture was then washed with saturated NaHCO₃, brine, dried (Na₂SO₄), filtered, and the filtrate concentrated under vacuum. The residue was purified by chromatography on silica (hexane/acetone 6:4) to yield (36) (306 mg, 77%).

R_{f}=0.52 (EtOAc); ¹H NMR (300 MHz, CDCl₃): δ 1.86 (8, 3 H, NH COCH₃), 1.99, 2.04, 2.07, 2.09, 2.14 (5s, 15H, COCH₃), 3.84-3.95 (m, 1H, H-9), 3.93 (s, 3H, COOCH₃), 4.12 (d, 1 H, H-3, J_{3,4}=9.6 Hz), 4.21-4.33 (m, 2H, H-5, H-6), 4.39 (d, 2H, CH₂CN), 4.48 (dd, 1H, J_{9′,8}=2.4 Hz, J_{9′,9}=12.3 Hz, H-9′), 4.95 (s, 1H, OH), 5.14 (m, 2H, H-4, H-8), 5.31 (dd, 1H, J_{7,6}=1.5 Hz, J_{7,8}=5.1 Hz, H-7), 6.05 (d, 1 H, NH); LRMS (ESI): m/z 569.1 [M+Na)⁺].

### Example 41

### Methyl 5-acetamido-2,4,7,8,9-penta-O-acetyl-3,5-dideoxy-3-O-acetonitrile-D-erythro-β-L-gluco-non-2-ulopyranosonate (37)

Compound (36) (300 mg, 0.55 mmol) was dissolved in dry pyridine (3 mL) and acetic anhydride (2 mL) and DMAP (catalytic amount) were added to the reaction mixture. After stirring for 16 h the reaction mixture was concentrated and the residue was purified by chromatography on silica (hexane/acetone 5:5) yielding (37) (314 mg, 97%).

R_{f}=0.48 (EtOAc); ¹H NMR (300 MHz, CDCl₃): δ 1.86 (s, 3H, NH COCH₃), 2.01, 2.04, 2.06, 2.09, 2.11, 2.14 (6s, 18H, COCH₃), 3.81 (s, 3H, COOCH₃), 4.02 (dd, 1H, J_{3,4}=6.6 Hz, J_{9,9′}=12.3 Hz, H-9), 4.12 (d, 1H, H-3, J_{3,4}=9.6 Hz), 4.12-4.23 (m, 2H, H-5, H-6), 4.29 (d, 1H, CH₂CN, J=16.8 Hz), 4.42 (dd, 1H, J_{9′,8}=2.7 Hz, (J_{9′,9}=12.3 Hz, H-9′), 4.49 (d, 1H, CH₂CN, J=16.8 Hz), 5.03-5.10 (m, 1H, H-8), 5.14 (dd, 1H, J_{4,5}=J_{4,3}=11.1 HZ, H-4), 5.32 (dd, 1H, J_{7,6}=1.8 Hz, J_{7,9}=6.0 Hz, H-7), 6.02 (d, 1H, J_{NH,5}=9.4 Hz, NH); LRMS (ESI): m/z 611.2 [M+Na)⁺].

### Example 42

### Methyl 5-acetamido-2,4,7,8,9-penta-O-acetyl-3,5-dideoxy-3-O-(2′-azidoethyl)-D-erythro-α-L-gluco-non-2-ulopyranosonate (39)

To a mixture of (37) (130 mg, 0.22 mmol) and Pd/C (10%, 125 mg) in methanol (3 mL) was added 1 M HCl solution (0.3 mL, 0.3 mmol). The mixture was stirred and shaken for 16 h at room temperature under a pressure of 40 psi of hydrogen. The reaction mixture was filtered through Celite and the filtrate concentrated under vacuum. The crude product (38) [R_{f}=0.42 (EtOAc/MeOH/H₂O 7:2:1)] was employed without further purification for the next reaction.

A solution of triflic azide in pyridine was prepared according to the method of Yan et al. [Ri-Bai Yan et al. Tetrahedron Lett. (2005) 46, 8993-8995]. Compound (38) (0.28 mg, 0.445 mmol) was dissolved in anhydrous pyridine (1.5 mL), then CuSO₄ (3 mg, 0.011 mmol) and triethylamine (124 microL, 0.89 mmol) were added and the solution was cooled to 5° C. The solution of TfN₃ (0.8 mL, 0.534 mmol) in anhydrous pyridine was added to the reaction mixture dropwise. After stirring at 5° C. for 10 min, the reaction was allowed to warm to room temperature and stirred for a further 16 h. The solvent was removed under vacuum and the crude product was purified by chromatography on silica (hexane/acetone 6:5) yielding (39) (357 mg, 77%).

R_{f}=0.45 (EtOAc); ¹H NMR (300 MHz, CDCl₃): δ 1.85 (s, 3H, NH COCH₃), 2.01, 2.03, 2.06, 2.09, 2.10, 2.15 (6s, 18H, COMA, 3.20-3.26 (m, 2H, OCH₂CH₂N₃), 3.65-3.76 (m, 2H, OCH₂CH₂N₃), 3.79 (s, 3H, COOCH₃), 3.85 (d, 1H, H-3, J_{3,4}=9.6 Hz), 4.02-4.10 (m, 2H, H-6, H-9), 4.19-4.31 (m, 1H, H-5), 4.46 (dd, 1H, J_{9′,8}=2.7 Hz, J_{9′,8}=12.6 Hz, H-9′), 5.00-5.04 (m, 1H, H-8), 5.18 (dd, 1H, J_{4,5}=J_{4,3}=9.9 Hz, H-4), 5.32 (dd, 1H, J_{7,8}=2.1 Hz, J_{7,8}=5.1 Hz, H-7), 5.49 (d, 1H, J_{NH,5}=9.9 Hz, NH); LRMS (ESI): m/z 641.1 [M+Na)⁺].

### Example 43

### Methyl 5-acetamido-4,7,8,9-tetra-O-acetyl-2,6-anhydro-3,5-dideoxy-3-O-(2′-azidoethyl)-D-glycero-D-galacto-non-2-enonate (41)

Compound (39) (60 mg, 0.097 mmol) was dissolved in anhydrous DCM (1 mL) under N₂ and the solution cooled to 0° C., when TMSBr (64 microL, 0.48 mmol) was added. The reaction mixture was allowed to warm to room temperature at which temperature it was stirred for 56 h. The reaction was concentrated under vacuum, yielding the crude glycosyl bromide (40) as white-yellow solid [R_{f}=0.48 (EtOAc)]. Compound (40) was used without purification for the elimination reaction. Crude (40) (0.097 mmol) was dissolved in anhydrous 1,2-dichloroethane (2 mL) under N₂ and the solution cooled to 0° C. when DBU (75 microL, 0.480 mmol) was added. The reaction was stirred overnight at room temperature then concentrated under vacuum. The residue was dissolved in EtOAc and washed with a saturated aqueous solution of NH₄Cl, water and brine and dried over Na₂SO₄, and concentrated under vacuum. The residue was purified by chromatography on silica (hexane/acetone 65:35) to give compound (41) (5 mg, 9%) as a white foam. Unreacted (39) (49 mg, 82%) was also recovered.

R_{f}=0.46 (EtOAc); ¹H NMR (300 MHz, CDCl₃): δ 2.04 (s, 3H, NH COCH₃), 2.06, 2.08, 2.10, 2.12 (4s, 12H, COCH₃), 3.40-3.53 (m, 2H, OCH₂CH₂N₃), 3.79 (s, 3H, COOCH₃), 3.86-3.96 (m, 1H, OCH₂CH₂N₃), 4.06-4.18 (m, 2H, OCH₂CH₂N₃, H-9), 4.28 (dd, 1H, J_{6,7}=3.6 Hz, J_{6,5}=8.7 Hz H-6), 4.36-4.45 (m, 1H, H-5), 4.54 (dd, 1H, J_{9′,8}=3.0 Hz, J_{9′,9}=12.6 Hz, H-9′), 5.25-5.31 (m, 1H, H-8), 5.48 (dd, 1H, J_{7,6}=3.9 Hz, J_{7,8}=5.4 Hz, H-7), 5.69 (d, 1H, J_{NH,5}=9.3 Hz, NH); 5.79 (d, 1H, J_{4,5}=6.9 Hz, H-4); LRMS (ESI): m/z 581.3 [M+Na)⁺].

### Example 44

### Methyl 5-acetamido-2,4,7,8,9-penta-O-acetyl-3,5-dideoxy-3-O-[2′-(4″-isobutyl-[1″,2″,3″]triazol-1″-yl)ethyl]-D-erythro-α-L-gluco-non-2-ulopyranosonate (42; R=2-methylpropyl) (42)

Compound (39) (100 mg, 0.162 mmol) and 2-methyl-4-pentyne (24 microL, 0.194 mmol) were dissolved in aqueous isopropanol solution (3 mL, isopropanol/H₂O 1:1). A 1 molar solution of copper(II) sulfate pentahydrate (32 microL, 0.032 mmol) was added, followed by the addition of 1M sodium ascorbate solution (64 microL, 0.065 mmol). The reaction was heated at 50° C. for 4 h. The mixture was evaporated under reduced pressure, and the residue was diluted with ethyl acetate and washed with water and brine, dried (Na₂SO₄), filtered, and the filtrate evaporated under vacuum. The residue was purified by chromatography on silica (hexane/acetone 4:6) yielding compound (42; R=2-methylpropyl) (103 mg, 91%) as a white solid.

R_{f}=0.07 (EtOAc); ¹H NMR (300 MHz, CDCl₃): δ 0.90 (d, 3H, CH₃, J=6.6 Hz), 0.91 (d, 3H, CH₃, J=6.6 Hz) 1.84 (s, 3 H, NH COCH₃), 1.87, 2.00, 2.03, 2.09, 2.18 (5s, 15H, COCH₃), 1.85-1.92 (m, 1H, CH(CH₃)₂), 2.54 (m, 2H, CH₂CH), 3.77 (s, 3H, COOCH₃), 3.84 (d, 1H, H-3, J_{3,4}=9.6 Hz), 3.86-4.08 (m, 4H, OCH₂CH₂N, H-6, H-9,), 4.09-4.19 (m, 1H, H-5), 4.23-4.48 (3H, OCH₂CH₂N, H-9′), 4.98-5.04 (m, 1 H, H-8), 5.12 (dd, 1H, J_{4,5}=J_{4,3}=13.5 Hz, H-4), 5.32 (dd, 1H, J_{7,6}=2.1 Hz, J_{7,6}=5.1 Hz, H-7), 5.49 (d, 1H, J_{NH,5}=9.6 Hz, NH), 7.25 (s, 1H, CHC); LRMS (ESI): m/z 723.3 [M+Na)⁺].

### Example 45

### Methyl 5-acetamido-4,7,8,9-tetra-O-acetyl-2,6-anhydro-3,5-dideoxy-3-O-[2′-(4″-isobutyl-[1″,2″,3″]triazol-1″-yl)ethyl]-D-glycero-D-galacto-non-2-enonate (44; R=2-methylpropyl) (44)

Compound (42) (71 mg 0.101 mmol) was dissolved in anhydrous 1,2-dichloroethane (2 mL) under N₂ and the solution cooled to 0° C., then AcBr (300 microL, 4.0 mmol) was added dropwise. MeOH, (80 microL, 2 mmol) was added slowly to the solution and the mixture was stirred for 1 h at 0° C. and then for a further 56 h at room temperature. The solution was diluted with anhydrous toluene and evaporated under reduced pressure. Evaporation with toluene was repeated a further 2 times to give the crude glycosyl bromide (43) as a yellow solid. Compound (43) was used without purification for the elimination reaction. Crude (43) (0.101 mmol) was dissolved in anhydrous 1,2-dichloroethane (2 mL) under N₂ and the solution cooled to 0° C. when DBU (61 microL, 0.404 mmol) was added. The reaction was stirred overnight at room temperature and then concentrated under vacuum. The residue was dissolved in EtOAc and washed with a saturated aqueous solution of Na₄Cl, water and brine, dried (Na₂SO₄), filtered, and the filtrate concentrated under vacuum. The residue was purified by chromatography on silica (EtOAc/dichloromethane 65:35) to give the compound (44; R=2-methylpropyl) (46 mg, 66%) as a white foam. Unreacted (42) (15 mg, 20%) was also recovered.

R_{f}=0.1 (EtOAc); ¹H NMR (300 MHz, CDCl₃): δ 0.90 (d, 6H, CH₃, J=6.6 Hz), 1.90 (s, 3H, NH COCH₃), 1.97, 2.02, 2.03, 2.09 (4s, 15H, COCH₃), 1.86-1.95 (m, 1H, CH(CH₃)₂), 2.54 (d, 2H, J=7.2 Hz CH₂CH), 3.72 (a, 3H, COOCH₃), 4.14 (dd, 1H, J_{9,8}=6.9 Hz, J_{9,9′}=12.3 Hz, H-9), 4.20-4.32 (m, 3H, OCH₂CH₂N, H-6), 4.31-4.41 (m, 1H, H-5), 4.54-4.59 (3H, OCH₂CH₂N, H-9′), 5.23-5.29 (m, 1H, H-8), 5.46 (dd, 1H, J_{7,6}=J_{7,8}=4.5 Hz, H-7), 5.65 (d, 1H, J_{4,5}=6.6 Hz, H-4), 5.97 (d, 1H, J_{NH,5}=9.3 Hz, NH), 7.36 (s, 1H, CHC); LRMS (ESI): m/z 663.4 [M+Na)^{+]}.

### Biological Data

### Example 46

Enzyme inhibition: Inhibition data against influenza A virus N1 and N2 sialidases for compounds (7), (9b-d) and (21), compared to parent template Neu5Ac2-en, is described in Table 1. Sialidase inhibition assays were carried-out on MES-β-dodecyl-D-maltoside cell extracts prepared from 293T cells transiently expressing the viral enzyme according to the known method (Rameix-Welti et al., 2006). Enzymatic activity was measured using the fluorogenic substrate 2-□lpha-(4′-methylumbelliferyl)-N-acetylneuraminic acid according to the known method (Potier et al., 1979). To measure the inhibitory effects of compounds, cells were preincubated for 30 min at 37° C. in the presence of variable concentrations of the compounds. The Kᵢ was calculated by fitting the data to the appropriate Michaelis Menten equations. The compounds with the C-3 side-chain (X₂), (7), (9b-d) and (21), show selective inhibition of N1 over N2 sialidases; in contrast the parent compound Neu5Ac2en (X₂═H) shows equivalent inhibition of both sialidases.

TABLE 1
Neu5Ac2en
In vitro inhibition of influenza A virus N1 and
N2 sialidases by compounds (7) and (9b-d) and (21)
compared to parent template
Neu5Ac2en.

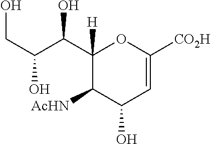

| | | Ki (microM)^{[a]} | |
|---|---|---|---|
| Compound | [X₂] | N1^{[b]} | N2^{[d]} |
| (7) | [allyl] | 222 ± 17 | 3629 ± 2130 |
| (9b) | [cyclohexyl-allyl] | 243 ± 21^{[c]} | >5000 |
| (9c) | [phenyl-allyl] | 52.1 ± 0.2^{[c]} | 1020 ± 40 |
| (9d) | [p-Me-phenyl-allyl] | 7.3 ± 0.8 | 219 ± 30 |
| (2l) | [4-OEt-3-allyl] | 810 ± 308 | >5000 |
| Neu5Ac2en | [H] | 0.97 ± 0.15 | 1.24 ± 0.23 |

^{[a]}Results are given as means ± SD for at least three independent determinations for duplicate samples.
^{[b]}N1 [A/HongRong/156/97 (H5N1)]
^{[c]}N1 [A/Cambodia/408/05 (H5N1)]
^{[d]}N2 [A/Paris/908/97 (H3N2)]

### Example 47

Enzyme inhibition: Inhibition of wild type and mutant (H274Y, N2948 and Q136K) influenza A virus N1 sialidases by (7) and (9d) compared to parent template Neu5Ac2en is described in Table 2. The H274Y, N294S and Q136K mutations were each introduced into a plasmidic clone encoding the N1 of A/Hong Kong/156/97, according to the known method (Rameix-Welti et al., 2006). Sialidase inhibition assays were carried-out as described in Example 46.

Mutations H274Y (which significantly reduces sensitivity to anti-influenza drug oseltamivir carboxylate [Okomo-Adhiambo et al. Antiviral Res. (2010) 85, 381] and N294S, both of which affect binding interactions in the main active site, affect similarly sensitivity to inhibition by (7), (9d), and parent template Neu5Ac2en. The Q136K mutation, which reduces sensitivity to the anti-influenza drug zanamivir [Okomo-Adhiambo et al. Antiviral Res. (2010) 85, 381], significantly increases sensitivity to compounds (7) and (9d).

TABLE 2
In vitro inhibition of wild type and mutant influenza A virus N1 sialidases
by (7) and (9d) compared to parent template Neu5Ac2en.

| | Kᵢ (microM)^{[a]} | | |
|---|---|---|---|
| N1 sialidase mutant | (7) | (9d) | Neu5Ac2en |
| [A/HongKong/156/97 | 222 ± 17 | 7.3 ± 0.8 | 0.97 ± 0.15 |
| (H5N1)] Wild type | | | |
| [A/HongKong/156/97 | 459 ± 92 | 11 ± 0.9 | 1.43 ± 0.17 |
| (H5N1)] H274Y | | | |
| [A/HongKong/156/97 | 980 ± 50 | 25 ± 3.1 | 3.9 ± 0.5 |
| (H5N1)] N294S | | | |
| [A/HongKong/156/97 | 16.8 ± 2.5 | 2.6 ± 0.4 | 1.37 ± 0.15 |
| (H5N1)] Q136K | | | |

^{[a]}Results are given as means ± SD for at least three independent determinations for duplicate samples.

### Example 48

Cell-based virus inhibition assay (Plaque Reduction Assay): In vitro sensitivity of influenza virus isolates to (7) and (9d), in comparison to parent template Neu5Ac2en, is shown in Table 3. The plaque phenotype of the indicated viruses was assayed on MDCK-SIAT cells (Matrosovich et al, 2003) in the presence of serial dilutions of (7) (500 nM to 5 mM), (9d) (10 nM to 1 mM), or Neu5Ac2en (10 nM to 1 mM), using a plaque assay protocol adapted from a published procedure (Matrosovich et al, 2006). Cells were stained with crystal violet after 72 h of incubation at 35° C. For each inhibitor, the average plaque diameters were plotted against the inhibitor concentrations. The 50% effective concentration (EC₅₀) was determined graphically as the concentration of inhibitor that induced a 50% reduction in the average plaque diameter.

Mirroring the results for sialidase inhibition (Examples 46 and 47), the compounds with the C-3 side-chain (X₂) (7) and (9d) selectively inhibit growth of the influenza viruses that express an N1 sialidase (H₁N₁), compared to the N2-expressing virus (H₃N₂). In contrast, the parent compound, C-3 unsubstituted Neu5Ac2en (X₂═H), shows equivalent growth inhibition of both viruses.

TABLE 3
In vitro sensitivity of influenza virus isolates to (7) and (9d),
in comparison to standard Neu5Ac2en.

| | EC₅₀ (microM)^{[a]} | | |
|---|---|---|---|
| Virus | (7) | (9d) | Neu5Ac2en |
| A/Paris/0497/2007 | 1800 | 80 | 20 |
| (H1N1, H274)^{[b]} | | | |
| A/Paris/1170/2007 | 2200 | 40 | 8 |
| (H1N1, Y274)^{[c]} | | | |
| A/Paris/908/97 | >5000 | >1000 | 20 |
| (H3N2) | | | |

^{[a]}EC₅₀ values were determined as the concentrations which induced a 50% reduction of the average plaque diameter in a single plaque reduction assay experiment.
^{[b]}H1N1 clinical isolate of the 2007-08 season sensitive to oseltamivir carboxylate.
^{[c]}H1N1 clinical isolate of the 2007-08 season naturally resistant to oseltamivir carboxylate.

In growth of A/Paris/0497/2007 (H1N1) virus, compound (9d) produced significant decrease in plaque size moving from 1 to 10 to 100 microM concentration. In contrast, in growth of A/Paris/908/97 (H3N2) virus, compound (9d) produced little or no visible difference in plaque size at 1, 10, or 100 microM concentration. Neu5Ac2en showed a similar decrease in plaque size for both viruses size moving from 1 to 10 to 100 microM concentration.

### Structural Data

### Example 49

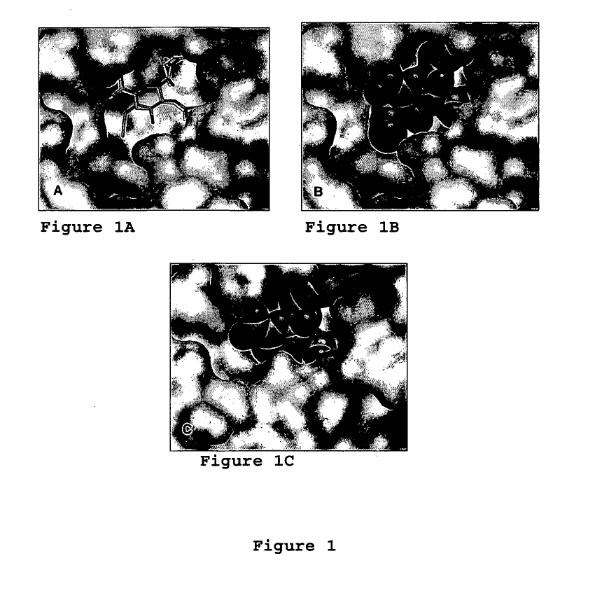

X-Ray crystallographic study of influenza A virus N8 sialidase-inhibitor complex: A group 1 (N8) influenza A virus sialidase crystal, prepared as previously described (Russell et al., 2006), was soaked in 1 mM solution of compound (7) for 60 minutes. The N8/(7) complex (FIGS. 1A and 1B) has the ‘open’ conformation of the 150-loop, in contrast to the N8 complex with C-3 unsubstituted Neu5Ac2en (FIG. 1C) where the 150-loop is ‘closed’ (Russell et al., 2006). The 3-allyl-Neu5Ac2en complex maintains the ‘open’ conformation of the 150-loop seen in the apo structure (Russell et al., 2006), with the C-3 alkyl side-chain of (7) bound into the 150-cavity as anticipated.

### Example 50

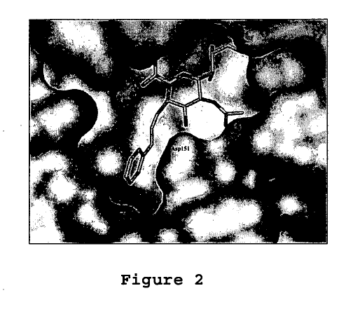

X-Ray crystallographic study of influenza A virus N8 sialidase-inhibitor complex: A group 1 (NB) influenza A virus sialidase crystal, prepared as previously described (Russell et al., 2006), was soaked in 1 mM compound (9c) for 60 minutes. The N8/(9c) complex (FIG. 2) has the ‘open’ conformation of the 150-loop with the C-3 phenylallyl substituent extending into the 150-cavity.

### Example 51

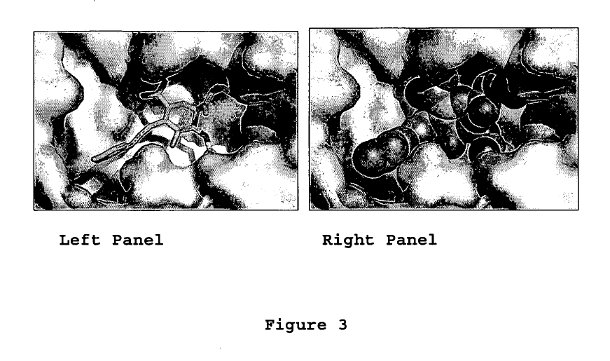

X-Ray crystallographic study of influenza A virus N8 sialidase-inhibitor complex: A group 1 (N8) influenza A virus sialidase crystal, prepared as previously described (Russell et al., 2006), was soaked in 1 mM compound (9d) for 60 minutes. The N8/(9d) complex (FIG. 3, left and right panels) has the ‘open’ conformation of the 150-loop with the C-3 (p-tolyl)allyl substituent extending well into the 150-cavity.

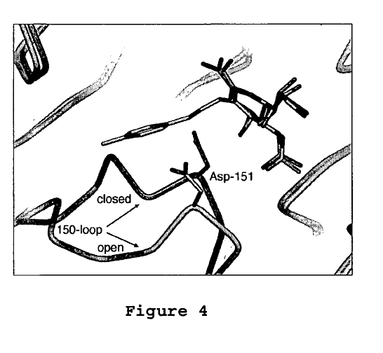

Superimposition of influenza A virus N8 X-ray crystal structures, open 150-loop N8/(9d), and the closed 150-loop in N8/Neu5Ac2en (PDB: 2 htr), is shown in FIG. 4. The dihydropyran ring and C-2, C-4, C-5, and C-6 substituents of (9d) and Neu5Ac2en have very similar positions in the active site. The phenyl ring of (9d) is positioned adjacent to Asp-151 in the open 150-loop conformation indicating the potential for interaction with this residue by suitable functionality (X₂) extending from the C-3 position of Neu5Ac2en or the corresponding position of other compositions of the invention.

### REFERENCES

Chapleur, Y. “Carbohydrate Mimics”, Wiley-VCH, Weinheim, 1998.
Douglas R. G. J. N. Engl. J. Med. (1990) 322, 443-450
Ernst, B.; Hart, G. W.; Sinay, P. (Eds), “Carbohydrates in Chemistry and Biology”, Wiley-VCH, Weinheim, 2000, Vols 1-4.
Falck-Pedersen, M. L.; Undheim, K. Tetrahedron (2005) 61, 6829-2835.
Florio, P.; Thomson, R. J.; Alafaci, A.; Abo, S.; von Itzstein, M. Bioorg. Med. Chem. Lett. (1999), 9, 2065-2068.
Florio, P.; Thomson, R. J.; von Itzstein, M. Carbohydr. Res. (2000), 328, 445-448.
Gyóllal, V.; Schanzenbach, D.; Somsák, L.; Linker, T. Chem. Commun. (2002) 1294-1295.
Kiefel, M. J.; von Itzstein, M. Chem. Rev. (2002) 102, 471-490.
Kim, C. U.; Lew, W.; Williams, M. A.; Liu, H.; Zhang, L.; Swaminathan, S.; Bischofberger, N.; Chen, M. S.; Mendel, D. B.; Tai, C. Y.; Layer, W. G.; Stevens, R. C. J. Am. Chem. Soc. (1997) 119, 681-690.
Kok, G. B.; Groves, D.; von Itzstein, M. J. Chem. Soc., Perkin Trans. 1 (1999) 2109-2115.
Linker, T. J. Organomet. Chem. (2002) 661, 159-167.
Mann, M. C.; Thomson, R. J.; Dyason, J. C.; McAtamney, S.; von Itzstein, M. Bioorg. Med. Chem. (2006) 14, 1518-1537.
Matrosovich, M.; Matrosovich, T.; Carr, J.; Roberts, N. A.; Klenk, H.-D. J. Virol. (2003) 77, 8418-8425.
Matrosovich, M.; Matrosovich, T.; Garten, W.; Klenk, H. D. Virol. J. (2006) 3, 63.
Meinke, S.; Thiem, J. Carbohydr. Res. (2008) 343, 1824-1829.
Okamoto, K.; Kondo, T.; Goto, T. Bull. Chem. Soc. Jpn. (1987) 60, 631-636.
Palese, P.; Compans R. W. J. Gen. Virol. (1976) 33, 159-163.
Paulsen, H.; Matschulat, P. Liebigs Ann. Chem. (1991) 487-495.
Potier, M.; Mameli, L.; Belisle, M.; Dallaire, L.; Melancon, S. B. Anal. Biochem. (1979) 94, 287-296.
Rameix-Welti, M. A.; Agou, F.; Buchy, P.; Mardy, S.; Aubin, J. T.; Veron, M.; van der Wert, S.; Naffakh, N. Antimicrob. Agents Chemother. (2006) 50, 3809-3815.
Reece, P. A. J. Med. Virol. (2007) 79, 1577-1586.
Rich J. R., Gehle D., von Itzstein M. (2007) “Design and synthesis of sialidase inhibitors for influenza virus infections.” In Comprehensive Glycoscience. Kamerling J. P., Boons G.-J., Lee Y. C., Suzuki A., Tanigichi N., Voragen A. G. J. (Bds); Elsevier, Oxford; pp 885-922.
Roy, R. Top. Curr. Chem. (1997), 187, 241-274.
Russell, R. J.; Haire, L. P.; Stevens, D. J.; Collins, P. J.; Lin, Y. P.; Blackburn, G. M.; Hay, A. J.; Gamblin, S. J.; Skehel, J. J. Nature (2006) 443, 45-49.
Smith, P. W.; Robinson, J. E.; Evans, D. N.; Sollis, S. L.; Howes, P. D.; Trivedi, N.; Bethell, R. C. Bioorg. Med. Chem. Lett. (1999) 9, 601-604.
Stick, R. V. “Carbohydrates—The sweet molecules of life”, Academic Press, San Diego, 2001.
Varghese, J. N.; McKimm-Breschkin, J. L.; Caldwell, J. B.; Kortt, A. A.; Colman, P. M. Proteins (1992) 14, 327-332.
von Itzstein, M.; Thomson, R. J. Top. Curr. Chem. (1997) 186, 119-170.
von Itzstein, M.; Wu, W-Y.; Kok, G. B.; Pegg, M. S.; Dyason, J. C.; Jin, B.; Phan, T. V.; Smythe, M. L.; White, H. F.; Oliver, S. W.; Colman, P. M.; Varghese, J. N.; Ryan, D. M.; Woods, J. M.; Bethell, R. C.; Hotham, V. J.; Cameron, J. M.; Penn, C. R. Nature (1993) 363, 418-423.
Zbiral, E. “Synthesis of sialic acid analogues and their behaviour towards the enzymes of sialic acid metabolism and hemagglutinin X-31 of influenza A-virus.” In Carbohydrates. Synthetic methods and applications in medicinal chemistry. Ogura, H.; Hasegawa, A.; Suami, T (Ede); VCH, Weinheim; 1992, pp 304-339.

1. A compound of formula (I) which is a selective inhibitor of influenza A virus group 1 sialidases: 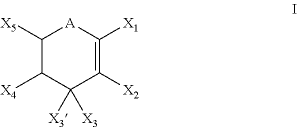
or a pharmaceutically acceptable salt, ester or prodrug thereof, wherein
A is O, S or NR₁;
where R₁ is hydrogen, optionally substituted alkyl, optionally substituted aryl, optionally substituted acyl or optionally substituted sulfonyl;
X₁ is CO₂H, P(O)(OH)₂, NO₂, SO₂H, SO₃H, —C(O)NHOH or tetrazole;
X₂ is alkyl, aralkyl, alkenyl, alkynyl, optionally substituted alkyl, optionally substituted aralkyl, optionally substituted alkenyl, optionally substituted alkynyl, OR₂, SR₂, NR₂R₂′, or substituted triazole,
where R₂ and R₂′ are selected independently from optionally substituted acyl, optionally substituted sulfonyl, alkyl, alkenyl, alkynyl, optionally substituted alkyl, or optionally substituted alkenyl,
or R₂′ is hydrogen;
X₃ and X₃′ are selected independently from hydrogen, R₃, halogen, CN, OR₃, NR₃R₃′, NHC(NR₃)N(R₃)₂, N₃, SR₃, —O—CH₂—C(O)—NR₃R₃′, —O—CH₂—C(NH)—NR₃R₃′, —O—CH₂—C(S)—NR₃R₃′
and optionally substituted triazole,
or X₃ and X₃′ together are ═O, ═N—OR₃, or ═CH—R₃
where R₃ and R₃′ are selected independently from hydrogen, optionally substituted acyl, optionally substituted sulfonyl, alkyl, aralkyl, alkenyl, alkynyl, heteroalkyl, heterocyclyl, optionally substituted alkyl, optionally substituted aralkyl, optionally substituted alkenyl, —C(O)R₈ and —S(O)₂R₈,
where R₈ is selected from optionally substituted alkyl and optionally substituted alkenyl;
X₄ is NR₄R₄′, OR₄, SR₄, CH₂C(O)R₄, CH₂C(O)OR₄, CH₂C(O)NR₄R₄′, CHR₄NO₂, CHR₄CN, CHR₄R₄′, or CH₂NHR,
where R₄ and R₄′ are selected independently from hydrogen optionally substituted acyl, optionally substituted thioacyl, optionally substituted sulfonyl, alkyl, alkenyl, alkynyl, optionally substituted alkyl, optionally substituted aralkyl, optionally substituted alkenyl, optionally substituted heteroaryl, and optionally substituted heterocyclyl;
X₅ is optionally substituted alkyl, optionally substituted aralkyl, optionally substituted alkenyl, optionally substituted alkynyl, optionally substituted heteroaryl, optionally substituted heterocyclyl, —C(O)R₅, —CO₂R₅, —C(O)NR₅R₅′, —P(O)(OR₅)(OR₅), —P(O)(OR₅)(NR₅R₅), —P(O)(NR₅R₅′)₂, CN, OR₆, azide, NHR₆, NR₆R₆′, SR₆, or optionally substituted triazole,
where R₅ and R_{5′} are independently selected from hydrogen, optionally substituted alkyl, optionally substituted alkenyl, optionally substituted aryl, or heteroaryl, and
R₆ and R₆′ are independently selected from optionally substituted acyl, optionally substituted sulfonyl, optionally substituted alkyl, optionally substituted aralkyl, optionally substituted alkenyl, optionally substituted aryl, heteroaryl, or heterocyclyl.

2. A compound as claimed in claim 1 wherein A is O.

3. A compound as claimed in claim 1 wherein X₁ is CO₂H or P(O)(OH)₂ or an ester thereof.

4. A compound as claimed in claim 3 wherein X₁ is CO₂H.

5. A compound as claimed in claim 1 wherein X₂ is alkyl, aralkyl, alkenyl, optionally substituted alkyl, optionally substituted aralkyl or optionally substituted alkenyl.

6. A compound as claimed in claim 1 wherein X₂ is OR₂, SR₂, NR₂R_{2′}.

7. A compound as claimed in claim 1 wherein X₃′ is hydrogen and X₃ is selected from R₃, OR₃, NR₃R₃′, NHC(NR₃)N(R₃)₂, N₃, SR₃, and optionally substituted triazole,
where R₃ and R₃′ are independently selected from alkyl, alkenyl, alkynyl, optionally substituted alkyl, optionally substituted alkenyl, —C(O)R₈ or —S(O)₂R₈,
where R₈ is selected from optionally substituted alkyl and optionally substituted alkenyl.

8. A compound as claimed in claim 1 wherein X₄ is —NR₄R₄′, R₄ is optionally substituted acyl and R₄′ is hydrogen.

9. A compound as claimed in claim 8 wherein R₄ is acyl.

10. A compound as claimed in claim 1 wherein X₅ denotes CH₂YR₇, CHYR₇CH₂YR₇ or CHYR₇CHYR₇CH₂YR₇,
where Y is O, S, or NR₇′, and successive Y moieties in an X₅ group are the same or different, or
where the substituent YR₇ is ═O, ═N—OR₇, or ═CHR₇, or
where two adjacent YR₇ groups together form part of a ring structure which optionally includes at least one heteroatom selected from O, S and N and is optionally substituted; in particular, an epoxide, aziridine, 5 or 6 membered cyclic ether group,
and R₇ and R₇′ are independently selected from hydrogen, optionally substituted acyl, optionally substituted sulfonyl, —S(O)₂OH, —P(O)(OH)₂, optionally substituted alkyl, optionally substituted aralkyl, optionally substituted alkenyl, optionally substituted aralkyl, and optionally substituted alkenyl.

11. A compound of formula (II) which is a selective inhibitor of influenza A virus group 1 sialidases: 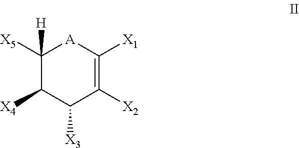
wherein X₁, X₂, X₃, X₄ and X₅ are as defined in claim 1.

12. A compound of formula (III) which is a selective inhibitor of influenza A virus group 1 sialidases: 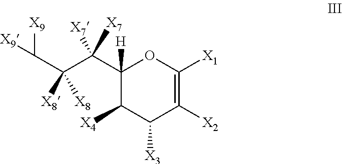
wherein X₁, X₂, X₃, and X₄ are as defined in claim 1 , provided that
one of X₇ and X₇′ is hydrogen,
one of X₈ and X₈′ is hydrogen,
one of X₉ and X₉′ is hydrogen, and
X₇, X₇′, X₈, X₈′, X₉, and X₉′ are the same or different, and are selected from H, OR_{S}, NR₇R₇′, SR_{S}, or optionally substituted triazole, or
together X₇ and X₇′, X₈ and X₈′, or X₉ and X₉′ form ═O, or ═N—OR_{S}.

13. A compound selected from the group consisting of:
methyl 5-acetamido-4,7,8,9-tetra-O-acetyl-2,6-anhydro-3,5-dideoxy-3-C-(prop-2′-enyl)-D-glycero-D-galacto-non-2-enonate,
5-acetamido-2,6-anhydro-3,5-dideoxy-3-C-(prop-2′-enyl)-D-glycero-D-galacto-non-2-en-onic acid,
methyl 5-acetamido-4,7,8,9-tetra-O-acetyl-2,6-anhydro-3,5-dideoxy-3-C-(4,4-dimethylpent-2′-enyl)-D-glycero-D-galacto-non-2-enonate,
5-acetamido-2,6-anhydro-3,5-dideoxy-3-C-(4,4-dimethylpent-2′-enyl)-D-glycero-D-galacto-non-2-enonic acid,
methyl 5-acetamido-4,7,8,9-tetra-O-acetyl-2,6-anhydro-3,5-dideoxy-3-C-(3′-cyclohexyl-prop-2′-enyl)-D-glycero-D-galacto-non-2-enonate,
5-acetamido-2,6-anhydro-3,5-dideoxy-3-C-(3′-cyclohexyl-prop-2′-enyl)-D-glycero-D-galacto-non-2-enonic acid,
methyl 5-acetamido-4,7,8,9-tetra-O-acetyl-2,6-anhydro-3,5-dideoxy-3-C-(3′-phenyl-prop-2′-enyl)-D-glycero-D-galacto-non-2-enonate,
5-acetamido-2,6-anhydro-3,5-dideoxy-3-C-(3′-phenyl-prop-2′-enyl)-D-glycero-D-galacto-non-2-enonic acid,
methyl 5-acetamido-4,7,8,9-tetra-O-acetyl-2,6-anhydro-3,5-dideoxy-3-C-[3′-(p-tolyl)-prop-2′-enyl]-D-glycero-D-galacto-non-2-enonate (8d, R=4-CH₃Ph),
5-acetamido-2,6-anhydro-3,5-dideoxy-3-C-[3′-(phenyl)-prop-2′-enyl]-D-glycero-D-galacto-non-2-enonic acid,
methyl 5-acetamido-4,7,8,9-tetra-O-acetyl-2,6-anhydro-3,5-dideoxy-3-C-[3′-(4-tert-butoxyphenyl)-prop-2′-enyl]-D-glycero-D-galacto-non-2-enonate,
5-acetamido-2,6-anhydro-3,5-dideoxy-3-C-[3′-(4-tert-butoxyphenyl)-prop-2′-enyl]-D-glycero-D-galacto-non-2-enonanic acid,
methyl 5-acetamido-4,7,8,9-tetra-O-acetyl-2,6-anhydro-3,5-dideoxy-3-C-(3′-naphthyl-prop-2′-enyl)-D-glycero-D-galacto-non-2-enonate,
5-acetamido-2,6-anhydro-3,5-dideoxy-3-C-(3′-naphthyl-prop-2′-enyl)-D-glycero-D-galacto-non-2-enonic acid,
methyl 5-acetamido-4,7,8,9-tetra-O-acetyl-2,6-anhydro-3,5-dideoxy-3-C-[4′-(3,4-dimethoxyphenyl)-but-2′-enyl]-D-glycero-D-galacto-non-2-enonate,
5-acetamido-2,6-anhydro-3,5-dideoxy-3-C-[4′-(3,4-dimethoxyphenyl)-but-2′-enyl]-D-glycero-D-galacto-non-2-enonic acid,
methyl 5-acetamido-3-C-(3′-acetoxypropyl)-4,7,8,9-tetra-O-acetyl-2,6-anhydro-3,5-dideoxy-D-glycero-D-galacto-non-2-en-onate,
5-acetamido-3-C-(3′-hydroxypropyl)-2,6-anhydro-3,5-dideoxy-D-glycero-D-galacto-non-2-enonic acid,
methyl 5-acetamido-4,7,8,9-tetra-O-acetyl-2,6-anhydro-3,5-dideoxy-3-C-propyl-D-glycero-D-galacto-non-2-enonate,
5-acetamido-2,6-anhydro-3,5-dideoxy-3-C-propyl-D-glycero-D-galacto-non-2-enonic acid,
methyl 5-acetamido-4,7,8,9-tetra-O-acetyl-2,6-anhydro-3,5-dideoxy-3-C-propenyl-D-glycero-D-galacto-non-2-enonate,
methyl 5-acetamido-2,6-anhydro-3,5-dideoxy-3-C-(prop-2′-enyl)-D-glycero-D-galacto-non-2-enonate,
methyl 5-acetamido-2,6-anhydro-3,5-dideoxy-8,9-O-isopropylidene-3-C-(prop-2′-enyl)-D-glycero-D-galacto-non-2-enonate,
methyl 5-acetamido-2,6-anhydro-3,5-dideoxy-4-O-ethyl-8,9-O-isopropylidene-3-C-(prop-2′-enyl)-D-glycero-D-galacto-non-2-enonate,
methyl 5-acetamido-2,6-anhydro-3,5-dideoxy-4-O-ethyl-8,9-O-isopropylidene-3-C-(prop-2′-enyl)-D-glycero-D-galacto-non-2-enonate,
5-acetamido-2,6-anhydro-3,5-dideoxy-4-O-ethyl-3-C-(prop-2′-enyl)-D-glycero-D-galacto-non-2-enonic acid,
2-methyl-(methyl 7,8,9-tri-O-acetyl-2,6-anhydro-3,5-dideoxy-3-C-(prop-2′-enyl)-D-glycero-D-talo-non-2-enonate)-[4,5-d]-2-oxazoline,
methyl 5-acetamido-7,8,9-tri-O-acetyl-2,6-anhydro-4-azido-3-C-(prop-2′-enyl)-3,4,5-trideoxy-D-glycero-D-galacto-non-2-enonate,
methyl 5-acetamido-4,7,8,9-tetra-O-acetyl-2,6-anhydro-3,5-dideoxy-3-O-ethyl-D-glycero-D-galacto-non-2-enonate,
5-acetamido-2,6-anhydro-3,5-dideoxy-3-O-ethyl-D-glycero-D-galacto-non-2-enonic acid,
methyl 5-acetamido-4,7,8,9-tetra-O-acetyl-2,6-anhydro-3,5-dideoxy-3-O-(2′-azidoethyl)-D-glycero-D-galacto-non-2-enonate, and
methyl 5-acetamido-4,7,8,9-tetra-O-acetyl-2,6-anhydro-3,5-dideoxy-3-O [2′-(4″-isobutyl-[1″,2″,3″]triazol-1″-yl)ethyl]-D-glycero-D-galacto-non-2-enonate.

14. A compound which is a multivalent presentation of any one or compounds as claimed in claim 1 comprising a plurality of said compounds bound through a linker to a multivalent template.

15. A pharmaceutical composition comprising a compound of as claimed in claim 1 and a pharmaceutically acceptable carrier.

16. A method of preventing or treating influenza in a subject comprising administering to said subject a compound as claimed in claim 1.

17. (canceled)

18. (canceled)

19. A method of preparing a compound of general formula (I) as claimed in claim 1:
1) providing a compound of formula (IV), wherein:
X₂, X₃, X₄ and X₅ are as defined in claim 1,
and may be protected by protecting groups,
X₆ is X₁, or a functional group that can be modified to form X₁, where X₆ can be selected from, but is not limited to, CHO, CN, CH₂OR′, thiazole, and
and Z is a group that can be activated to enable beta-elimination; 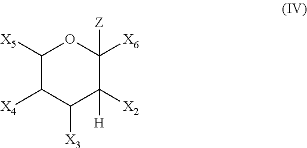
2) eliminating H—Z from the compound of general formula (IV);
3) converting X₆ to X₁ when it is other than X₁;
4) optionally functionalizing X₁, X₂, X₃, X₄ and/or X₅; and
5) optionally deprotecting X₁, X₂, X₃, X₄ and/or X₅.

20. A method as claimed in claim 19 wherein:
Z is a halide and elimination is achieved under basic conditions; or
Z is a halide and elimination is achieved in the presence of a heavy metal reagent;
or
Z is acyloxy and elimination is achieved under Lewis acidic conditions; or
Z is alkoxy and elimination is achieved under acetolysis conditions; or
Z is phosphite and elimination is achieved under Lewis acidic conditions.

21. A method of preparing a compound of general formula (I) as claimed in claim 1, comprising the steps of:
1) providing a compound of general formula (V), 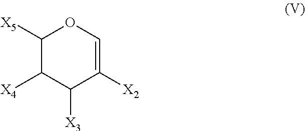
wherein X₂, X₃, X₄ and X₅ are as defined and may be protected by protecting groups;
2) introducing X₁ to the compound of general formula (V) in a direct C-1 lithiation followed by reaction of the lithiated species with EX₁ wherein E is an electrophile and X₁ may be protected with a protecting group;
3) optionally functionalizing X₁, X₂, X₃, X₄ and/or X₅; and
4) optionally deprotecting X₁, X₂, X₃, X₄ and/or X₅.
